(19) United States
(12) Patent Application Publication
(10) Pub. No.: US 2026/0031182 A1
(43) Pub. Date: Jan. 29, 2026

(54) METHOD TO DETERMINE A PREDOMINANT IMMUNE SIGNAL IN A BREAST CANCER MICROENVIRONMENT

(71) Applicant: THE BOARD OF REGENTS OF THE UNIVERSITY OF TEXAS SYSTEM, Austin, TX (US)

(72) Inventors: Isaac CHAN, Dallas, TX (US); Lily XU, Dallas, TX (US); Kaitlyn SAUNDERS, Dallas, TX (US)

(73) Assignee: THE BOARD OF REGENTS OF THE UNIVERSITY OF TEXAS SYSTEM, Austin, TX (US)

(21) Appl. No.: 18/993,459

(22) PCT Filed: Jul. 14, 2023
(86) PCT No.: PCT/US2023/070269

§ 371 Date: Jan. 10, 2025

Related U.S. Application Data
(60) Provisional application No. 63/389,725, filed on Jul. 15, 2022.

(51) Int. Cl. G16B 25/10; G16H 20/10; G16H 50/20

(52) U.S. Cl. CPC G16B 25/10; G16H 20/10; G16H 50/20

(57) ABSTRACT

Disclosures herein are directed to methods for classifying and characterizing cancer epithelial cells and analyzing their level of interaction with secondary cell populations in order to identify, modify or otherwise tailor immunotherapy and treatment modalities to a patient. Also disclosed are methods of identifying suitable patient candidates for immunotherapy treatments as well as methods of identifying targets of immunotherapies for the treatment and/or prevention of cancer.

### CROSS REFERENCE TO RELATED APPLICATION

This application claims the benefit of U.S. Provisional Application No. 63/389,725 filed on Jul. 15, 2022, the disclosure of which is hereby incorporated by reference in its entirety.

### BACKGROUND

### 1. Field

The present inventive concept is directed to methods of classifying populations of tumor cells and related secondary cells and to identify and treat suitable subjects in need with immunotherapy.

### 2. Discussion of Related Art

While immunotherapy has revolutionized the treatment of many solid tumors, the efficacy of immunotherapy regimens are comparatively lower in breast cancer. Immunotherapy efficacy is often negatively correlated with intratumor heterogeneity. Novel immunotherapy approaches in breast cancer should leverage how cancer epithelial cell heterogeneity affects immune cells in the tumor microenvironment. However, current definitions of cancer epithelial cell heterogeneity in breast cancer have limited resolution. Single cell RNA-seq (scRNA-seq) provides an unprecedented opportunity to further define cancer epithelial cell heterogeneity and identify how heterogeneity influences interactions with immune cells. New methods of classifying intratumor heterogeneity to identify optimal candidates for immunotherapy are needed.

### SUMMARY

The present disclosure is based, in part, on the novel finding that cancer epithelial cells can be classified based on their gene expression and analyzed for their level of interaction with secondary cell populations in order to predict responsiveness to therapeutics targeting the tumor microenvironment.

Accordingly, in some aspects, a method of classifying a cancer epithelial cell to a gene element group is provided, the method comprising: (i) obtaining a set of expressed genes in the cancer epithelial cell; (ii) determining expression levels of genes in a plurality of gene sets and rank the expression levels in each gene set to identify a gene set having highest gene expression; and (iii) assigning the cell to a gene element group corresponding to the gene set having highest gene expression.

In various aspects, the cancer epithelial cell is assigned to a gene element 1 group (GE1) when the gene set having highest gene expression comprises at least one gene, at least 5 genes, at least 10 genes, at least 15 genes, or at least 20 genes selected from the group consisting of AC090498-1, AC105999-2, ADIRF, AGR2, AGR3, ALDH2, ANKRD30A, ARL6IP1, ARMT1, ATAD2, AZGP1, BATF, BMPR1B, BST2, BTG2, C15ORF48, CCDC74A, CEBPD, CFD, CLDN4, CLU, COX6C, CPB1, CRIP1, CST3, CTHRC1, CXCL14, DHRS2, DSCAM-AS1, ELF3, ELP2, ERBB4, ESR1, EVL, FABP3, FHL2, FKBP5, FSIP1, GJA1, GSTM3, HES1, HSPB1, IFI27, IFI6, IFITM1, IFITM2, IFITM3, IGFBP4, INPP4B, ISG15, JUNB, KCNE4, KCNJ3, KRT18, KRT19, LDLRAD4, MAGED2, MDK, MESP1, MGP, MGST1, MRPS30, MRPS30-DT, MS4A7, MT-ATP8, NOVA1, PEG10, PHGR1, PI15, PIP, PLAAT4, PLAT, PRSS23, PSD3, PVALB, RAMP1, RBP1, RHOBTB3, SCGB3A1, SCUBE2, SEMA3C, SERPINA1, SH3BGRL, SLC39A6, SLC40A1, SNCG, STC2, TCEAL4, TCIM, TFF1, TFF3, TIMP1, TMC5, TPM1, TPRG1, VSTM2A, VTCN1, WFDC2, XBP1, and ZFP36L1.

In various aspects, the cancer epithelial cell is assigned to a gene element 2 group (GE2) when the gene set having highest gene expression comprises at least one gene, at least 5 genes, at least 10 genes, at least 15 genes, or at least 20 genes selected from the group consisting of ALDH3B2, ALOX15B, APOD, AZIN1, B2M, BNIP3, C1orf21, CALD1, CALU, CAPG, CD24, CD59, CD74, CD99, CDKN2B, CFD, CKB, CLDN3, CLDN4, CNN3, COL12A1, COX6C, CRIP1, CSRP1, CSRP2, CTNNB1, CTTN, CYSTM1, DDIT4, DHRS2, DLX5, DSC2, EFHD1, EFNA1, ELF5, ENO1, FAM229B, FASN, GJA1, GRIK1-AS1, GSTP1, H2AJ, HILPDA, HNRNPH1, HSPA5, IFI27, IFITM3, IGKC, JPT1, KCNC2, KRT15, KRT23, KRT7, LAPTM4B, LDHB, LMO4, LTF, MAFB, MAL2, MAOB, MFAP2, MGST1, MRPL15, MT1X, MUCL1, MYBPC1, NME2, NUPR1, PCSK1N, PFN2, PHGDH, PRSS23, PSMB3, PTHLH, PTPN1, RAMP1, RAMP3, RBP1, RSU1, S100A10, S100A6, SCUBE2, SFRP1, SH3BGRL, SLC39A4, SLC40A1, SOX4, STC2, STOM, TCIM, TFF3. TMSB4X, TTYH1, TUBA1A, UBE2V2, VIM, YBX1, YBX3, YWHAH, and YWHAZ.

In various aspects, the cancer epithelial cell is assigned to a gene element 3 group (GE3) when the gene set having highest gene expression comprises at least one gene, at least 5 genes, at least 10 genes, at least 15 genes, or at least 20 genes selected from the group consisting of A2M, ACTA2, ACTG2, ANGPTL4, ANXA1, APOD, APOE, BGN, C6ORF15, CALD1, CALML5, CAV1, CAVIN1, CAVIN3, CCL28, CCN2, CD24, CDKN2A, CHI3L1, COL1A2, COL6A1, COL6A2, COTL1, CRYAB, CSTA, CXCL2, DEFB1, DEPP1, EFEMP1, FABP5, FBXO32, FDCSP, FGFBP2, FN1, GABRP, GSTP1, HLA-A, HLA-B, ID1, IFI27, IGFBP3, IGFBP5, IGFBP7, IL32, KLK5, KLK7, KRT14, KRT15, KRT16, KRT17, KRT5, KRT6A, KRT6B, KRT81, LAMB3, LCN2, LTF, LY6D, MFAP5, MFGE8, MGP, MIA, MMP7, MT1X, MT2A, MYL9, MYLK, NDRG1, NDUFA4L2, NFKBIA, NNMT, PDLIM4, PLS3, POSTN, PRNP, PTN, RARRES1, RCAN1, RGS2, S100A2, S100A4, S100A6, S100A8, S100A9, SAA1, SAA2, SBSN, SERPING1, SFRP1, SGK1, SLC25A37, SLPI, SPARC, SPARCL1, TAGLN, THBS1, TPM2, TSHZ2, VIM, and ZFP36L2.

In various aspects, the cancer epithelial cell is assigned to a gene element 4 group (GE4) when the gene set having highest gene expression comprises at least one gene, at least 5 genes, at least 10 genes, at least 15 genes, or at least 20 genes selected from the group consisting of ANLN, ANP32E, ARL6IP1, ASF1B, ASPM, ATAD2, AURKA, BIRC5, BUB1B, CCNB1, CCNB2, CDC20, CDC6, CDCA3, CDCA8, CDK1, CDKN2A, CDKN3, CENPA, CENPE, CENPF, CENPK, CENPM, CENPU, CENPW, CIP2A, CKAP2, CKLF, CKS1B, CKS2, CTHRC1, DEK, DLGAP5, DTYMK, DUT, ECT2, FAM111A, FAM111B, GGH, GTSE1, H1-2, H1-3, H2AZ1, H2AZ2, H2BC11, H4C3, HELLS, HMGB1, HMGB2, HMGB3, HMGN2, HMMR, IQGAP3, KIF20B, KIF23, KIF2C, KNL1, KPNA2, LGALS1, MAD2L1, MKI67, MT2A, MYBL2, MZT1, NEK2, NUF2, NUSAP1, PBK, PCLAF, PCNA, PLK1, PRC1, PRR11, PTTG1, RACGAP1, RAD21, RHEB, RNASEH2A, RPL39L, RRM2, SMC4, SPC25, STMN1, TFDP1, TK1, TMEM106C, TMPO, TOP2A, TPX2, TROAP, TTK, TUBA1B, TUBA1C, TUBB, TUBB4B, TYMS, UBE2C, UBE2S, UBE2T, and ZWINT.

In various aspects, the cancer epithelial cell is assigned to a gene element 5 group (GE5) when the gene set having highest gene expression comprises at least one gene, at least 5 genes, at least 10 genes, at least 15 genes, or at least 20 genes selected from the group consisting of AIF1, ALOX5AP, ANXA1, APOC1, APOE, AREG, C1ORF162, C1QA, C1QB, C1QC, CARD16, CCL3, CCL4, CCL5, CD2, CD27, CD37, CD3D, CD3E, CD48, CD52, CD53, CD69, CD7, CD74, CD83, CELF2, COL1A2, CORO1A, CREM, CST7, CTSL, CTSW, CXCR4, CYBB, CYTIP, DUSP2, EMP3, FCER1G, FN1, FYB1, GIMAP7, GMFG, GPR183, GPSM3, GZMA, GZMK, HCST, HLA-DPA1, HLA-DPB1, HLA-DQA1, HLA-DRA, HLA-DRB1, IGSF6, IL2RG, IL32, IL7R, ISG15, ITGB2, KLRB1, LAPTM5, LCK, LIMD2, LSP1, LST1, LTB, LY96, LYZ, MEF2C, MNDA, MS4A6A, MSR1, NKG7, PTPRC, RAC2, RGCC, RGS1, RGS2, RNASE1, S100A4, S100A6, SEPTIN6, SLC2A3, SMAP2, SOCS1, SPARC, SPP1, SRGN, STK4, TMSB4X, TNFAIP3, TRAC, TRBC1, TRBC2, TREM2, TYROBP, VIM, WIPF1, ZEB2, and ZNF331.

In various aspects, the the cancer epithelial cell is assigned to a gene element 6 group (GE6) when the gene set having highest gene expression comprises at least one gene, at least 5 genes, at least 10 genes, at least 15 genes, or at least 20 genes selected from the group consisting of ADIRF, ANAPC11, ATP5ME, AZGP1, BLVRB, BST2, CALM1, CCND1, CD9, CETN2, CISD3, CLDN7, COX6C, CRABP2, CRACR2B, CRIP1, CRIP2, CSTB, CYB5A, CYBA, CYC1, DBI, DCXR, DSTN, EEF1B2, ELOC, EMP2, FXYD3, GPX4, GSTM3, H2AJ, H2AZ1, HINT1, HMGB1, HSPE1, IDH2, JPT1, KDELR2, KRT10, KRT18, KRT19, KRT7, KRT8, LGALS1, LGALS3, LSM3, LSM4, LY6E, MARCKSL1, MIEN1, MIF, MPC2, MRPL12, MRPL51, MRPS34, MTDH, MUCL1, NDUFB9, NDUFC2, NME1, PAFAH1B3, PFDN2, PFN1, PIP, POLR2K, PPDPF, PSMA7, PSMB3, PSME2, RAN, RANBP1, RBIS, REEP5, ROMO1, RPS26, S100A14, S100A16, SEC61G, SELENOP, SH3BGRL, SLC9A3R1, SMIM22, SNRPB, SNRPG, SPINT2, SQLE, SRP9, STARD10, TCEAL4, TMCO1, TMEM14B, TPI1, TPM1, TSPAN13, TUBA1B, TUBB, UQCRQ, XBP1, YBX1, and ZNF706.

In various aspects, the cancer epithelial cell is assigned to a gene element 7 group (GE7) when the gene set having highest gene expression comprises at least one gene, at least 5 genes, at least 10 genes, at least 15 genes, or at least 20 genes selected from the group consisting of AC093001-1, ADIRF, AGR2, AGR3, ANKRD37, APOD, AQP3, ARC, AREG, ATF3, AZGP1, BAMBI, BTG1, BTG2, C15ORF48, CALML5, CCDC74A, CCN1, CD55, CDKN1A, CEBPB, CEBPD, CFD, CLDN3, CLDN4, CST3, CTD-3252C9-4, CTSK, DHRS2, DNAJB1, DUSP1, EDN1, EGR1, ELF3, ELOVL2, ESR1, FHL2, FOS, FOSB, GATA3, GDF15, GRB7, GSTM3, H1-2, HES1, ICAM1, ID2, IER2, IER3, IFITM1, IGFBP4, IGFBP5, IRF1, JUN, JUNB, KLF4, KLF6, KRT15, KRT18, LGALS3, MAFB, MAGED2, MGP, NAMPT, NCOA7, NFKBIA, NFKBIZ, NR4A1, NR4A2, PERP, PLAT, PMAIP1, PRSS23, REL, RHOV, RND1, S100P, SAT1, SLC39A6, SLC40A1, SOCS3, SOX4, SOX9, STC2, TACSTD2, TCIM, TFF1, TIMP3, TM4SF1, TNFRSF12A, TSC22D3, TUBA1A, VASN, VEGFA, VTCN1, XBP1, ZFAND2A, ZFP36, ZFP36L1, and ZFP36L2.

In various aspects, the cancer epithelial cell is assigned to a gene element 8 group (GE8) when the gene set having highest gene expression comprises at least one gene, at least 5 genes, at least 10 genes, at least 15 genes, or at least 20 genes selected from the group consisting of ADIRF, AFF3, ALCAM, ANKRD30A, ANXA2, AR, ARFGEF3, ASAH1, ATP1B1, AZGP1, BTG1, CD59, CDK12, CEBPD, CLDN3, CLDN4, CLTC, CLU, CNN3, CTNNB1, CTNND1, EFHD1, EGR1, ELF3, EPCAM, ERBB2, ESR1, EVL, FOSB, GATA3, GRB7, H4C3, HES1, HLA-B, HNRNPH1, HSPA1A, HSPA1B, IGFBP5, INTS6, ITGB1, ITGB6, ITM2B, JUN, KLF6, KRT7, LDLRAD4, LMNA, LRATD2, MAGED2, MAL2, MARCKS, MT-ND4L, MT2A, MUC1, MYH9, NEAT1, NFIB, PERP, PKM, PLAT, PMEPA1, PSAP, RAD21, RBP1, RHOB, RUNX1, S100A10, SAT1, SCARB2, SCD, SDC1, SERHL2, SH3BGRL3, SHISA2, SLC38A2, SLC39A6, SLC40A1, SOX4, SYTL2, TACSTD2, TCAF1, TCIM, TFAP2B, TIMP1, TM4SF1, TMC5, TMEM123, TPM1, TRPS1, TSC22D1, TSPYL1, TUBA1A, VEGFA, WSB1, XIST, YBX1, YBX3, ZFP36L1, ZFP36L2, and ZNF292.

In various aspects, the cancer epithelial cell is assigned to a gene element 9 group (GE9) when the gene set having highest gene expression comprises at least one gene, at least 5 genes, at least 10 genes, at least 15 genes, or at least 20 genes selected from the group consisting of AC093001-1, ADIRF, AGR2, AGR3, APOD, AQP1, AQP5, AREG, ASCL1, AZGP1, BMPR1B, C15ORF48, CALML5, CCL28, CD55, CEACAM6, CFD, CLIC3, CLU, COX6C, CSTB, CTSD, CXCL14, CXCL17, DHRS2, DSCAM-AS1, DUSP1, ERBB2, FADS2, FAM3D, FHL2, GDF15, GLYATL2, GPX1, GSN, GSTP1, HDC, HSPB1, IGFBP5, ISG20, ITM2A, KRT23, KRT7, LGALS1, LGALS3, LY6E, MARCKS, MFGE8, MGP, MS4A7, MT-ATP8, MTCO2P12, MUC5B, MUCL1, NDRG2, NFKBIZ, NPW, NR4A1, NUDT8, PALMD, PDZK1IP1, PERP, PHGR1, PIP, PLAT, PRSS21, PSCA, PTHLH, PYDC1, RGS10, RGS2, RHCG, RP11-53019-2, S100A1, S100A10, S100A6, S100A7, S100A8, S100A9, S100P, SAA2, SCGB1D2, SCGB2A1, SCGB2A2, SDC2, SERHL2, SERPINA1, SLC12A2, SLC18A2, SLPI, SYNM, TACSTD2, TFF1, TFF3, TM4SF1, TMC5, TSC22D3, TSPAN1, TXNIP, and XBP1.

In various aspects, the cancer epithelial cell is assigned to a gene element 10 group (GE10) when the gene set having highest gene expression comprises at least one gene, at least 5 genes, at least 10 genes, at least 15 genes, or at least 20 genes selected from the group consisting of AGR2, APOD, AREG, AZGP1, B2M, BST2, BTG2, C15ORF48, CCL20, CD74, CEBPD, CHI3L1, CHI3L2, CP, CRISP3, CSTA, CTSC, CTSD, CTSS, CXCL1, CXCL17, CYBA, DEFB1, FDCSP, GBP1, GBP2, HLA-A, HLA-B, HLA-C, HLA-DMA, HLA-DPA1, HLA-DPB1, HLA-DQA1, HLA-DQA2, HLA-DQB1, HLA-DRA, HLA-DRB1, HLA-DRB5, HLA-E, ID3, IFI16, IFI27, IFI44L, IFI6, IFIT1, IFIT2, IFIT3, IFITM1, IFITM2, IFITM3, IGFBP7, IL32, IRF1, ISG15, KRT15, KRT19, KRT5, KRT7, LCN2, LGALS1, LGMN, LTF, LUM, LY6D, LYZ, MAFB, MARCKS, MGP, MIA, MMP7, MRPS30-DT, MX1, NNMT, PI3, PIGR, RAMP2, RARRES1, RHCG, RNASE1, RSAD2, S100A8, S100A9, S100P, SAA2, SCGB1D2, SCGB2A1, SERPING1, SLC39A6, SOD2, SPATS2L, TCIM, TFF1, TFF3, TMEM45A, TNFAIP6, TNFSF10, TXNIP, WFDC2, XBP1, and ZFP36.

Further aspects of the present disclosure are directed to a method of determining a level of intra-tumoral heterogeneity (ITH) in a tumor, the method comprising classifying a plurality of cancer epithelial cells in the tumor to a gene element group as described herein.

Further aspects of the present disclosure are directed to a method of classifying an immune cell to an immune cell subset, the method comprising: (i) obtaining a set of expressed genes in the immune cell; (ii) determining expression levels of genes in a plurality of gene sets and rank the expression levels in each gene set to identify a gene set having highest gene expression; and (iii) assigning the immune cell to an immune cell subset corresponding to the gene set having highest gene expression.

In some aspects, the immune cell is a natural killer cell (NK cell) and the immune cell subset is a NK cell subset, wherein the natural killer cell is classified to an NK-0 subset, when the gene set having highest gene expression comprises at least one gene selected from the group consisting of FCGR3A, PRF1, FGFBP2, GZMH, and ETS1. In other aspects, the natural killer cell is classified as an NK-1 cell when the gene set having highest gene expression comprises at least one gene selected from the group consisting of NR4A1, NR4A2, DUSP1, DUSP2, FOS, and JUN. In other aspects, the natural killer cell is classified as an NK-2 cell when the gene set having highest gene expression comprises at least one gene selected from the group consisting of FCGR3A, PRF1, FGFBP2, GZMA, GZMB, CXCF1, SPON2, CX3CR1, and S1PR5. In some aspects, the natural killer cell is classified as an NK-3 cell when the gene set having highest gene expression comprises at least one gene selected from the group consisting of GZMK, SELL, IL7R, and LTB. In still other aspects, the natural killer cell is classified as an NK-4 cell when the gene set having highest gene expression comprises at least one gene selected from the group consisting of ISG15, IFI6, IFIT3, and IFI44L. In still other aspects, the natural killer cell is classified as an NK-5 cell when the gene set having highest gene expression comprises at least one gene selected from the group consisting of CCL5, HLA-DRB1, KLRC1, CD74, MYADM, and HSPE1. In further aspects, the natural killer cell is classified as a reprogrammed NK cell (rNK cell), when the gene set having highest gene expression comprises at least one gene selected from the group consisting of ABCA1, ALOX12, CALD1, CAVIN2, CCL4, CLU, CMKLR1, CR2, CX3CR1, DTX1, DUSP1, F5, FAM81A, FOS, FOSB, GAS2L1, GFRA2, GP6, HEATR9, HES1, ITGAX, JUN, KLRG1, LTBP1, MID1, MPIG6B, NHSL2, NR4A1, NR4A2, NR4A3, NYLK, PARVB, PLXNA4, RASGRP2, RHPN1, SCD, SLC6A4, SLC7A5, THBS1, TMTC1, TNFAIP3, TUBB1, VWF, and XDH.

Further aspects of the present disclosure are directed to a method of determining a level of interaction between a tumor and a secondary cell population, the method comprising: (a) obtaining a population of cancer epithelial cells from the tumor; (b) assigning each cancer epithelial cell obtained in (a) to a gene element group according to the method of claim 1; (c) determining average expression of each gene element group in (b) across the population of cancer epithelial cells; (d) obtaining a set of prioritized receptor-ligand pairs across the tumor and the secondary cell population, each comprising a ligand expressed by a cancer epithelial cell assigned in step (b) and a prioritized receptor expressed by a secondary cell; (e) determining average expression of prioritized receptors from the set of prioritized receptor-ligand pairs in the secondary cell population; and (f) determining the level of interaction between the tumor and the secondary cell population based on the average expression of each gene element group in (c) and the average expression of prioritized receptors in (e).

In various aspects, the prioritized receptor-ligand pairs in (d) either increase or decrease an interaction between the cancer epithelial cell and the secondary cell and wherein the gene element groups in (b) are further classified as “activating” or “inactivating” based on ligand expression of cells assigned to each gene element group, wherein (1) cells assigned to an activating gene element group express ligands from prioritized receptor-ligand pairs that increase the level of interaction between the cancer epithelial cell and the secondary cells and (2) cells assigned to an inactivating gene element group express ligands from prioritized receptor-ligand pairs that decrease the level of interaction between the cancer epithelial cell and the secondary cell.

In some aspects, determining the level of interaction in (f) is positively weighted by average expression of gene element group classified as “activating” and negatively weighted by average expression of gene element groups classified as “inactivating”.

In further aspects, (f) can be calculated using an equation comprising: IP(Rᵢ)=Σᵢ₌₁¹⁰(eᵢ)(Rᵢ)(w), wherein i corresponds to each gene element group, eᵢ is average expression of each gene element group, Rᵢ is the number of prioritized receptors on the secondary cell type and w is positive 1 for an activating gene element group and negative 1 for an inactivating gene element group.

In further aspects, the level of interaction in (f) is further based on average levels of one or more interacting factors associated with the cancer epithelial cell population and/or the secondary cell population. In these aspects, the method comprises (i) determining the average level of at least one interacting factor and (ii) reclassifying the gene element groups in (b) as “activating” or “inactivating” wherein (1) cells classified in an activating gene element group are directly or indirectly acted upon by the interacting factor such that the level of interaction between the cancer epithelial cell and the secondary cells increases as levels of the interacting factor increase and (2) cells classified in an inactivating gene element group are directly or indirectly acted upon by the interacting factor such that the level of interaction between the cancer epithelial cell and the secondary cells decreases as levels of the interacting factor increase. In these aspects, an interacting score for determining the level of interaction in (f) is calculated using an equation comprising: IP=IP(Rᵢ)+IP(Bᵢ) wherein IP(Rᵢ) is calculated as described above and IP(Bᵢ) is calculated as: IP(Bᵢ)=Σᵢ₌₁¹⁰(eᵢ)(Bᵢ)(w_{Bi}) wherein i corresponds to each gene element group, eᵢ is average expression of each gene element group, Bᵢ is the average level of an interacting factor associated with the secondary cell population and w_{Bi} is positive 1 for an activating gene element group and negative 1 for an inactivating gene element group.

In various aspects, the level of interaction in (f) may be based on average levels of more than one interacting factors associated with the secondary cell population. In these instances, the level of interaction in (f) can be calculated using an equation comprising IP=IP(Rᵢ)+IP(B1ᵢ)+IP(B2ᵢ) . . . wherein each IP(Bᵢ) refers to an interacting score calculated as described above for each of the more than one interacting factor.

In any of these aspects, the one or more interacting factors can comprise an autocrine factor, a paracrine factor, a juxtacrine factor, or an endocrine factor. In some aspects, the one or more interacting factors comprise a cytokine, a chemokine, an extracellular matrix remodeling factor (MMP), a secreted peptide, a hormone, a neuromodulator, a growth factor, or a metabolic factor.

In any of the foregoing embodiments, the secondary cell can be an immune cell, a fibroblast, or an endothelial cell. In various aspects, the immune cell can be selected the group consisting of T cells, NK cells, B cells, and any other immune cell of a lymphocyte myeloid lineage. In further aspects, the immune cell can a reprogrammed NK cell classified according to the methods herein.

Further aspects are directed to a method of determining whether a subject with a tumor is a candidate for an immunotherapy, the method comprising: (i) determining a level of interaction between the tumor in the subject and a secondary cell according to a method provided above; and (ii) determining that the subject is a candidate for the immunotherapy if the level of interaction determined in (i) exceeds a threshold.

In some aspects, the secondary cell can be targeted by the immunotherapy or interact with a cell targeted by the immunotherapy.

Further aspects of the present disclosure are directed to a method of treating a patient with a tumor, the method comprising: (a) determining the subject is a candidate for immunotherapy according to a method herein; and (b) administering an immunotherapy to the subject. In various aspects, the immunotherapy is a T-cell directed therapy. In some aspects, the T-cell directed therapy alters immune cell activity, proliferation, and/or survival. In some aspects, immunotherapy can comprise an engineered cellular therapy, a small molecule inhibitor, a cytokine or hormone, an antibody-drug conjugate, a bi-specific antibody or a tri-specific antibody. In some aspects, the immunotherapy comprises a CAR-T cell, a CAR-NK cell, or an immune checkpoint inhibitor. In some aspects, the immune checkpoint inhibitor is an anti-PD-L1 therapy or anti-PD-1 therapy.

In various aspects, the tumor is a solid malignant tumor. In some aspects, the tumor is a breast cancer tumor.

In any of the foregoing or related aspects, the subject may be a canine or human.

Further aspects of the present disclosure are directed to a method for developing an immunotherapy, the method comprising: (a) obtaining one or more candidate cell sets for targeting with a potential immunotherapy, each candidate cell set comprising a population of tumor cells and a population of secondary cells that interact with or are suspected of interacting with the tumor cells; (b) determining a level of interaction between the tumor cells and the secondary cells in each candidate set according to a method provided herein; and (c) selecting a cell set having a level of interaction that exceeds a threshold for further development of an immunotherapy that alters or exploits the level of interaction between the cell populations in the selected set.

In various aspects, the population of secondary cells in each candidate cell set comprises immune cells. In various aspects, the immunotherapy is developed to increase activity of the immune cell population in the selected cell set and/or reduce immune suppression by the tumor cells in the selected cell set.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein. Embodiments of the present inventive concept are illustrated by way of example in which like reference numerals indicate similar elements.

FIG. 1 shows how integrated scRNA-seq dataset of primary breast cancer identifies heterogeneous cancer epithelial cells expressing clinically relevant targets. FIG. 1A depicts a brief overview of processing and integration pipeline for 8 primary breast cancer datasets. FIG. 1B depicts UMAP visualization of 236,363 cells across 119 samples from 88 patients analyzed by scRNA-seq. FIG. 1C depicts UMAP visualization showing major subsets of natural killer (NK) cells. FIG. 1D is a bubble heatmap showing expression of upregulated differentially expressed genes for each major NK cell subset (Bonferroni adjusted p-value<0.05). FIG. 1E is a Boxplot showing expression of the rNK cell signature in each NK cell subset. NK-1 was significantly different from all other clusters (Kruskal-Wallis p<0.0001, with post-hoc Dunn test p-values shown; ****p-value<0.0001). FIG. 1F is a MA plot of differentially expressed genes between rNK and non-rNK cells (Bonferroni adjusted p-value<0.05). FIG. 1G is a Boxplot showing the expression level of the rNK signature by clinical subtype. FIG. 1H depict Circos plots showing representative predictive receptor-ligand pairs between rNK cells and all cancer epithelial cells separated by clinical subtype. Shared receptors across all subtypes are colored in red. FIG. 1I depicts a boxplot showing the Pearson correlations of rNK signature gene expression in reprogrammed NK (rNK) cells compared to non-rNK cells versus rNK cells compared to rNK cells (across all clinical subtypes of breast cancer). Pearson correlations between rNK cells and rNK cells are higher than those between rNK cells and non-rNK cells (two-sided Wilcoxon test, ****p-value<0.0001). FIG. 1J shows a scatterplot showing the Pearson correlation of age and proportion of rNK cells by sample (p-value<0.01). FIG. 1K shows a Kaplan-Meier plot showing worse clinical outcome in breast cancer patients with high expression of the rNK cell gene signature (log-rank test, p-value<0.05). FIG. 1L shows a Bar plot showing relative proportions of NK subsets across tumor samples and clinical subtypes.

FIG. 2 shows data showing that cancer epithelial cells demonstrate substantial intratumoral transcriptional heterogeneity. FIG. 2A is a bar plot showing proportion of ERBB2^{Hi}, ERBB2^{Med}, and ERBB2^{Lo} cells by sample. FIG. 2B is a bar plot showing proportion of TACSTD2^{Hi}, TACSTD2^{Med}, and TACSTD2^{Lo} cells by sample. FIG. 2C is a heatmap of z-scored average expression of clinically actionable targets in ERBB2^{Hi}, ERBB2^{Med}, ERBB2^{Lo} cells. FIG. 2D is a heatmap of z-scored average expression of clinically actionable targets in TACSTD2^{Hi}, TACSTD2^{Med}, and TACSTD2^{Lo} cells. FIG. 2E depicts an MA plot showing differentially expressed genes between ERBB2^{Hi} vs. ERBB2^{Med} and ERBB2^{Lo} cells (Bonferroni adjusted p-value<0.05). FIG. 2F depicts an MA plot showing differentially expressed genes between TACSTD2^{Hi} vs. TACSTD2^{Med} and TACSTD2^{Lo} cells (Bonferroni adjusted p-value<0.05). FIG. 2G is a boxplot showing the proportion of ERBB2^{Hi} expressing cells per sample by nodal status (two-sided Wilcoxon test p-value>0.05). FIG. 2H is a boxplot showing the proportion of TACSTD2^{Hi} expressing cells per sample by nodal status (two-sided Wilcoxon test p-value<0.05). FIG. 2I shows the percentage of cancer epithelial cells by molecular subtype, sorted by sample score by the ROGUE metric. FIG. 2J is a plot showing discordance in predicted heterogeneity by molecular subtype and by ROGUE metric by sample. Samples with >50% difference between the normalized ROGUE metric and the maximum percentage of cells within the sample that belonged to a single molecular subtype are classified as discordant.

FIG. 3 shows data showing that cancer epithelial cell heterogeneity can be defined by 10 GEs which influence immune cell interactions. FIG. 3A depicts a heatmap of z-scored signature scores of the 10 identified gene elements (GEs) representing all cancer epithelial cells, ordered based on the maximum z-scored GE signature score. Annotations represent dataset origin, clinical subtype, PAM50 subtype, and SC50 subtype. FIG. 3B depicts percentage of cancer epithelial cells assigned to each GE by molecular subtype. FIG. 3C shows a gene set enrichment using ClusterProfiler of the differentially expressed genes by GE. Significantly enriched gene sets from the MSigDB HALLMARK collection are shown (Benjamini-Hochberg adjusted p-value<0.05). FIG. 3D depicts a heatmap of curated predicted receptor-ligand pairs between cancer epithelial cells by GE and interacting immune and stromal cells. FIG. 3E shows scatterplots showing Pearson correlations of expression of NK-cell related GE1 and sensitivity to NK cell killing (Benjamini-Hochberg adjusted p-value<0.0005). FIG. 3F depicts Circos plots showing curated receptor-ligand pairs between cancer epithelial cells that highly express NK cell-related GE1 and GE6 and NK cells. NK cell activating receptor-ligand pairs are colored blue; NK cell inactivating receptor-ligand pairs are colored red.

FIG. 4 shows data demonstrating that GE-immune interactions predict response to anti-PD-1 therapy. FIG. 4A shows a heatmap of Pearson correlations between expression of each of the 10 GEs and the presence of CD8+ T cells for 6 spatial transcriptomics samples across spots containing CD8+ T cells (n.s. Benjamini-Hochberg adjusted pvalue>0.05). FIG. 4B shows for a representative TNBC sample, pathological annotation of morphological regions into distinct categories. UCell signature scores of CD8+ T cells overlaid onto spatial tumor sample spots in red. UCell signature score of GE5 (a CD8+ T cell activating GE) overlaid onto tumor sample spots in red. Colocalization score for ITGB2ITGAL, LTB TNFRSF1A, and ALOX5APALOX5 (a predicted receptor-ligand pair for GE5) overlaid onto tumor sample spots in red. FIG. 4C depicts a heatmap of average expression of each of the 10 GEs across cancer epithelial cells in each sample from Bassez et al. T cell InteractPrint is shown below. FIG. 4D shows a boxplot showing T cell InteractPrint prediction of response to anti-PD-1 therapy across all clinical subtypes in Bassez et al. (R=Responder; NR=Non-responder; p-value<0.05). AUC of ROC comparing performance of T Cell InteractPrint (AUC=81.87) and of PD-L1 expression (AUC=49.71) in Bassez et al. samples (bootstrap test with n=10,000 p-value<0.05). FIG. 4E shows a heatmap of average expression of each of the 10 GEs across cancer epithelial cells in each sample from the ISPY2 trial. T cell InteractPrint is shown below. FIG. 4F shows a boxplot showing T cell InteractPrint prediction of response to anti-PD-1 therapy across all clinical subtypes in ISPY 2 trial samples (R=Responder; NR=Non-responder; two-sided Wilcoxon test p-value<0.0001). AUC of ROC comparing performance of T Cell InteractPrint (AUC=83.02) and of PD-L1 expression (AUC=72.33) in ISPY 2 trial (bootstrap test with n=10,000 p-value<0.05). FIG. 4G depicts a schema of T cell InteractPrint to predict patient response to anti-PD-1 therapy.

FIGS. 5A-5L depict metadata and quality control of integrated scRNA-seq data. FIG. 5A is a pie chart of composition of integrated scRNA-seq data by original study. FIG. 5B is a pie chart of composition of integrated scRNA-seq data by clinical subtype. The proportion of clinical subtypes within this integrated dataset is close to real-life clinical subtype distributions. FIG. 5C is a bar plot showing number of patients per age group. Most of the original datasets stayed within a sole age group, whereas the integrated dataset includes a much broader age range. FIG. 5D is a UMAP visualization of integrated dataset following batch correction, grouped by source dataset. FIG. 5E is a UMAP visualization of integrated dataset following batch correction, grouped by capture technology. FIG. 5F is a UMAP visualization of integrated scRNA-seq data grouped by clinical subtype. This shows lineage drives clustering of non-epithelial populations, while epithelial populations cluster by clinical subtype. This matches the observed subtype clustering seen in other datasets. FIG. 5G is a PCA plot of first 2 PCs for all cells in the integrated dataset following batch correction, labeled by original source dataset. No cluster is driven by a single study, thus confirming there is no batch effect due to different studies. FIG. 5H is a PCA plot of first PCs for all cells in the integrated dataset following batch correction labeled by technology. No cluster is driven by a single technology, thus confirming there is no batch effect due to differing technologies. FIG. 5I is a PCA plot of first 2 PCs for all cells in the integrated dataset following batch correction, labeled by clinical subtype. FIG. 5J shows violin plots of mean PC loadings across top 20 PCs for the integrated dataset following batch correction, stratified by source dataset. FIG. 5K shows violin plots of mean PC loadings across top 20 PCs for the integrated dataset following batch correction, stratified by capture technology. FIG. 5L shows Violin plots of mean PC loadings across top 20 PCs for the integrated dataset following batch correction, stratified by clinical subtype.

FIGS. 6A-6I show visualization of combined original source datasets prior to batch correction. FIG. 6A-6C show UMAP visualization of combined original source datasets prior to batch correction, grouped by source dataset (FIG. 6A), capture technology (FIG. 6B) or clinical subtype (FIG. 6C). FIG. 6D-6F show PCA plots of first 2 PCs for combined original source datasets prior to batch correction, labeled by source dataset (FIG. 6C), capture technology (FIG. 6D) or clinical subtype (FIG. 6E). FIG. 6G-6I show Violin plots of mean PC loadings across top 20 PCs for combined original source datasets prior to batch correction, stratified by source dataset (FIG. 6G), capture technology (FIG. 6H) or clinical subtype (FIG. 6I).

FIGS. 7A-7D show classification of epithelial cells as cancer versus normal using CNV profile analysis. FIG. 7A shows a scatter plot showing classification of epithelial cells in the integrated dataset as cancer (malignant) versus normal (non-malignant) on inferCNV signal (x-axis) and CNV correlation (y-axis). Thresholds shown in red dashed lines. CNV signal reflects the extend of CNVs, while CNV correlation reflects the similarity between the cellular CNV pattern and that of other cells from the same tumor. Cells assigned as cancer (malignant) are shown in blue, while the rest are shown in red. FIG. 7B shows UMAP visualization of all epithelial cells in the integrated dataset, grouped by classification as cancer (malignant) versus normal (non-malignant). Cancer cells are shown in blue, while normal cells are shown in red. Unassigned cells are shown as NAs and are colored grey. FIG. 7C shows a Scatter plot showing classification of epithelial cells in the Bassez et al. dataset as cancer (malignant) versus normal (non-malignant) on inferCNV signal (x-axis) and CNV correlation (y-axis). FIG. 7D shows a UMAP visualization of all epithelial cells in the Bassez et al. dataset, grouped by classification as cancer (malignant) versus normal (non-malignant). Cancer cells are shown in blue, while normal cells are shown in red.

FIGS. 8A-8E show data relating to unsupervised clustering of NK cells and analysis of NK cell subsets. FIG. 8A shows UMAP visualization of all NK cells in the integrated dataset, grouped by source dataset, patient and capture technology. FIG. 8B shows silhouette scores for clustering of NK cells at various resolutions (0.1, 0.2, 0.3, 0.4, 0.5, 0.6, 0.7, 1.0). Mean silhouette score is shown as a red dashed line. Maximum mean silhouette score was observed at resolution 0.1 (2 clusters), and second highest mean silhouette score was observed at resolution 0.3 (6 clusters). FIG. 8C shows feature plots showing expression of NK subset markers and functional NK cell genes across all NK cells in our integrated dataset. FIG. 8D shows MA plots showing differentially expressed genes between individual NK cell subsets and all other NK cell subset types (Bonferroni adjusted p-value<0.05). FIG. 8E shows gene set enrichment of the differentially expressed genes by each NK cell subset. Significantly enriched gene sets from the MSigDB HALLMARK collection are shown (Benjamini-Hochberg adjusted p-value<0.05).

FIGS. 9A-9G show data relating to rNK signature development and analysis. FIG. 9A shows a heatmap showing z-scores for the variance-stabilized transformed expression of differentially expressed genes between healthy NK cells and tumor-promoting NK cells from previous study. FIG. 9B depicts a bubble heatmap showing expression of upregulated and downregulated human rNK orthologs for each major NK cell subset. FIG. 9C depicts a Boxplot showing the expression level of the rNK signature by clinical subtype, stratified by age. No significant difference was found between subtypes (Kruskal-Wallis p>0.05). FIG. 9D depicts a Boxplot showing the Pearson correlations of rNK signature gene expression in reprogrammed NK (rNK) cells compared to non-rNK cells versus rNK cells compared to rNK cells, stratified by age. Pearson correlations between rNK cells and rNK cells are higher than those between rNK cells and non-rNK cells for both age strata (two-sided Wilcoxon test, ****p-value<0.0001). FIG. 9E is a scatterplot showing the Pearson correlation between age at initial diagnosis and survival across TCGA samples (p-value>0.01). FIG. 9F shows Kaplan-Meier plots evaluating the influence of rNK cell gene signature expression on survival outcomes in TCGA patients with relatively high fraction of NK cells, stratified by age. For patients ≥45 yo, high rNK cell gene signature expression is associated with worse survival outcomes (log-rank test, p-value<0.05). FIG. 9G shows a Boxplot showing heterogeneity calculated as 1-ROGUE score for NK cells in each sample by breast cancer clinical subtype (*p-value<0.05).

FIGS. 10A-10K show differential gene expression and gene set enrichment analyses for each ERBB2 and TACSTD2 population. FIGS. 10A-10C are a UMAP visualization of all epithelial cells in the integrated dataset, grouped by patient (FIG. 10A), capture technology (FIG. 10B) or source dataset (FIG. 10C). Consistent with other tumor type and breast tumor datasets, epithelial cells appear to cluster by patient. FIG. 10D is a Boxplot showing % ERBB2+ cells by clinical subtype across samples in the integrated dataset. As anticipated, % ERBB2+ cells were significantly enriched in HER2+ samples compared to HR+ and TNBC samples (Kruskal-Wallis p<0.05, with post-hoc Dunn test p values shown). FIG. 10E is a scatterplot showing the Pearson correlation between HER2+ protein expression and ERBB2 mRNA expression across TCGA samples (p<0.0001). FIG. 10F depicts gene set enrichment of the differentially expressed genes by ERBB2Hi, ERBB2Med, and ERBB2Lo cells. Significantly enriched gene sets from the MSigDB HALLMARK collection are shown (Benjamini-Hochberg adjusted p-value<0.05). FIG. 10G depicts gene set enrichment of the differentially expressed genes by TACSTD2Hi, TACSTD2Med, and TACSTD2Lo cells. Significantly enriched gene sets from the MSigDB HALLMARK collection are shown (Benjamini-Hochberg adjusted p-value<0.05) FIG. 10H is a MA plot showing differentially expressed genes between ERBB2Hi vs. ERBB2Med and ERBB2Lo cells (Bonferroni adjusted p-value<0.05). FIG. 10I is a MA plot showing differentially expressed genes between ERBB2Lo vs. ERBB2Hi and ERBB2Med cells (Bonferroni adjusted p-value<0.05). FIG. 10J is a MA plot showing differentially expressed genes between TACSTD2Med vs. TACSTD2Hi and TACSTD2Lo cells (Bonferroni adjusted p-value<0.05). FIG. 10K is a MA plot showing differentially expressed genes between TACSTD2Lo vs. TACSTD2Hi and TACSTD2Med cells (Bonferroni adjusted p-value<0.05).

FIGS. 11A-11C show analysis of clinical features and associations across samples in the integrated dataset and depict boxplots showing the proportion of TACSTD2-expressing cells (FIG. 11A), ERBB2-expressing cells (FIG. 11B) or TACSTD2-expressing cells (FIG. 11C) per sample by nodal status, split by clinical subtype (FIG. 11A and FIG. 11B) or original source dataset (FIG. 11C). All results were analyzed by two-sided Wilcoxon test p-value as shown. For FIG. 11C, the combined result was not a statistically significant finding, though it does trend toward significance (Fisher's combined probability test, X=11.227, p=0.08).

FIGS. 12A-12G show data related to the generation and characterization of the 10 gene elements of cancer epithelial cell heterogeneity and exploration in breast cancer cell lines. FIG. 12A shows Metrics used to select the number of clusters (10) for consensus clustering of signatures of cancer epithelial cell ITTH. FIG. 12B shows spherical k-means (skmeans) consensus clustering of the Jaccard similarities between signatures of cancer epithelial cell ITTH, showing the probability (p1-p10) of each generated signature of being assigned to one of 10 classes. Silhouette scores are shown for each class or GE. FIG. 12C shows a heatmap of average z-scored expression of each of the 10 GEs across cancer epithelial cells in each sample in our integrated dataset. FIG. 12D is a Heatmap of the absolute number of curated predicted receptor-ligand pairs between cancer epithelial cells by GE and interacting immune and stromal cells. FIG. 12E is a heatmap of average z-scored expression of each of the 10 GEs across 10 cell lines derived from primary breast tumors. Cell lines are annotated by molecular subtype (basal A, basal B, HER2-amplified). FIG. 12F shows Cytotoxicity of NK cells against HCC1954, MCF7, and K562 cell lines, assessed by % LDH produced at 12 h timepoint. HCC1954 highly expressed NK-resistance GEs (GE1 and GE6), while MCF7 has low expression of NK-resistance GEs. Cytotoxicity was significantly reduced for the HCC1954 cell line compared to the MCF7 cell line (3 biological replications; Benjamini-Hochberg adjusted, *p-value<0.05, ***p-value<0.001). FIG. 12G depicts scatterplots showing Pearson correlations of expression of GEs with limited predicted interactions with NK cells (all but GE1) and sensitivity to NK cell killing across 10 cell lines derived from primary breast tumors (Benjamini-Hochberg adjusted p-values>0.05).

FIGS. 13A-13B show data related to predicted GE-immune interactions and spatial analysis of the 10 gene elements FIG. 9A depicts a heatmap showing the proportion of spatial tumor sample spots within a sample that contain each of the GEs and immune or stromal cell populations. FIG. 9B depicts how for a representative TNBC sample, UCell signature scores of each GE overlaid onto spatial tumor sample spots with >10% presence of cancer epithelial cells.

FIGS. 14A-14B show unbiased clustering of NK cells from human pancreatic tumors, with 4 clusters represented (FIG. 14A) and then application of the reprogrammed NK (rNK) cell signature revealing cluster 2 correlates strongly with signature expression.

FIG. 15 shows percentage of cancer cell death of two cancer cell lines (MDA-MB-436 and BT-474) in co-cultures with NK cells (NK-92 cells) in the presence or absence of an anti-TIGIT blocking antibody.

### I. TERMINOLOGY

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

As used herein, the term “about,” can mean relative to the recited value, e.g., amount, dose, temperature, time, percentage, etc., ±10%, ±9%, ±8%, ±7%, ±6%, ±5%, ±4%, ±3%, ±2%, or ±1%.

The terms “comprising,” “including,” “encompassing” and “having” are used interchangeably in this disclosure. The terms “comprising,” “including,” “encompassing” and “having” mean to include, but not necessarily be limited to the things so described.

The terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

As used herein “immune checkpoint inhibitor” or “ICI” is a drug that blocks immune checkpoints. These checkpoints are a normal part of the immune system and keep immune responses from being too strong. By blocking them, these drugs allow immune cells to respond more strongly to cancer. Immune checkpoint inhibitors work by preventing cancer cells from turning T-cells (white blood cells that detect infections and abnormalities) off. Non-limiting examples of immune checkpoint inhibitors include inhibitors of PD-1, PD-L1, TIM-3, LAG-3, CTLA-4, and CSF-1R and any combination thereof. The immune checkpoint receptors may be on tumor cells or immune cells such as T cells, monocytes, microglia, and macrophages, without limitation. The agents which assert immune checkpoint blockade may be small chemical entities or polymers, antibodies, antibody fragments, single chain antibodies or other antibody constructs, including, but not limited to, bispecific antibodies and diabodies. Immune checkpoint inhibitors which may be used according to the disclosure include any that disrupt the inhibitory interaction of cytotoxic T cells and tumor cells. These include but are not limited to anti-PD-1 antibody, anti-PD-L1 antibody, anti-CTLA4 antibody, anti-LAG-3 antibody, anti-TIM-3 antibody. The inhibitor need not be an antibody but can be a small molecule or other polymer. If the inhibitor is an antibody it can be a polyclonal, monoclonal, fragment, single chain, or other antibody variant construct. Inhibitors may target any immune checkpoint known in the art, including but not limited to, CTLA-4, PDL1, PDL2, PD1, B7-H3, B7-H4, BTLA, HVEM, TIM3, GAL9, LAG3, CSF-1R, VISTA, KIR, 2B4, CD160, CGEN-15049, CHK1, CHK2, A2aR, CD28, CD86 and the B-7 family of ligands. Combinations of inhibitors for a single target immune checkpoint or different inhibitors for different immune checkpoints may be used. Illustrative examples of immune checkpoint inhibitors include CTLA-4 blocking antibodies (Ipilimumab (Yervoy), Tremelimumab (Imjuno)), PD-1 inhibitors (Pembrolizumab (Keytruda), Nivolumab (Opdivo), Cemiplimab (Libtayo), CT-011 (Pidilizumab), AMP224), PD-L1 inhibitors (Atezolizumab (tecentriq), Avelumab (Bavencio), Durvalumab (Imfinzi), BMS-936559), Lag3 inhibitors (Relatlimab), combination of Lag3 and PD1 inhibitor (PD-1 inhibitor nivolumab (Opdualag) OX40 inhibitor (MEDI6469), CD160 inhibitor (BY55). Non-limiting examples of inhibitors of CSF-1R include PLX3397, PLX486, RG7155, AMG820, ARRY-382, FPA008, IMC-CS4, JNJ-40346527, and MCS 110. The terms “ICI treatment”, “ICI therapy”, “ICI compounds”, and the like, refer to one or more ICI (or the use thereof) disclosed herein or known to those of skill in the art.

As used herein “immune cell” is a cell which develops from stem cells in the bone marrow and become different types of white blood cells. Immune cells include neutrophils, eosinophils, basophils, mast cells, monocytes, macrophages, dendritic cells, natural killer cells, and lymphocytes (B cells and T cells).

As used herein “cancer” may be one or more neoplasm or cancer. The neoplasm may be malignant or benign, the cancer may be primary or metastatic; the neoplasm or cancer may be early stage or late stage. Preferably, the neoplasm or cancer is a solid malignant cancer (e.g., a carcinoma, a sarcoma, or a lymphoma). Non-limiting examples of carcinomas, sarcomas or lymphomas include breast cancer, adrenocortical carcinoma, AIDS-related cancers, AIDS-related lymphoma, anal cancer, appendix cancer, astrocytoma (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sezary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, and Wilms tumor (childhood).

In various aspects, the cancer is a breast cancer and may identified histologically as ductal, lobular, invasive breast carcinoma, carcinoma with apocrine differentiation, metaplastic carcinoma, invasive lobular carcinoma surrounding soft tissue, invasive breast carcinoma-no special type with medullary pattern, Invasive Ductal Carcinoma NST, Invasive Lobular Carcinoma, Invasive Ductal Carcinoma, Invasive Apocrine Carcinoma, or a combination of any thereof.

As used herein, the terms “treat,” “treating,” “treatment,” and the like, unless otherwise indicated, can refer to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the condition, or disorder.

The term “biomolecule” as used herein refers to, but is not limited to, proteins, enzymes, antibodies, DNA, siRNA, and small molecules. “Small molecules” as used herein can refer to chemicals, compounds, drugs, and the like.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

It should also be understood that, unless clearly indicated to the contrary, in any methods claimed herein that include more than one step or act, the order of the steps or acts of the method is not necessarily limited to the order in which the steps or acts of the method are recited.

### II. METHODS OF CLASSIFYING CELLS

Various aspects of the present disclosure are directed to methods of classifying tumor and/or associated secondary cells (e.g., cells that interact with a tumor) and then applying these classifications to determine a score for a tumor in a subject, wherein the score can be used to predict effectiveness of a given immunotherapy in the subject. Current methods for evaluating candidates for immunotherapy rely on broad genotyping from a single sample from a tumor and do not account for intratumor heterogeneity which can reduce therapeutic efficacy. Therefore, the novel methods herein may be applied to identify previously unknown ideal candidates for various therapies.

In various aspects, methods herein provide for (a) classifying one or more cancer epithelial cells (e.g., from a single tumor). In other aspects, methods herein provide means for (b) classifying natural killer cells in a subject. Finally, one or more of these methods are then combined to allow for (c) classifying entire tumors (and consequently, a subject) as being suitable for targeting with certain therapeutics or treatments. Ultimately, the novel methods provided herein provide for a more granular approach to tumor classification and an improved ability to judge potential success of a given treatment regimen.

As used herein, a suitable subject includes a mammal, a human, a livestock animal, a companion animal, a lab animal, or a zoological animal. In some embodiments, a subject may be a rodent, e.g., a mouse, a rat, a guinea pig, etc. In other embodiments, a subject may be a livestock animal. Non-limiting examples of suitable livestock animals may include pigs, cows, horses, goats, sheep, llamas and alpacas. In yet other embodiments, a subject may be a companion animal. Non-limiting examples of companion animals may include pets such as dogs (canine), cats (feline), rabbits, and birds. In yet other embodiments, a subject may be a zoological animal. As used herein, a “zoological animal” refers to an animal that may be found in a zoo. Such animals may include non-human primates, large cats, wolves, and bears. In other embodiments, the animal is a laboratory animal. Non-limiting examples of a laboratory animal may include rodents, canines, felines, and non-human primates. In some embodiments, the animal is a rodent. Non-limiting examples of rodents may include mice, rats, guinea pigs, etc. In preferred embodiments, the subject is a human. In additional preferred embodiments, the subject is a canine.

In various aspects, the methods disclosed herein comprise obtaining a gene expression profile from one or more cells. As used herein, “obtaining a gene expression profile is used synonymously with “obtaining a set of expressed genes.” As used herein, the terms “gene expression profile” or “set of expressed genes” refer to a pattern of genes expressed by a cell at the transcription level. Non-limiting examples of methods of measuring gene expression in one or more cells suitable for use herein include high-density expression array, DNA microarray, polymerase chain reaction (PCR), reverse transcriptase PCR (RT-PCR), real-time quantitative reverse transcription PCR (qRT-PCR), digital droplet PCR (ddPCR), serial analysis of gene expression (SAGE), Spotted cDNA arrays, GeneChip, spotted oligo arrays, bead arrays, RNA Seq, tiling array, northern blotting, hybridization microarray, in situ hybridization, or any combination thereof. In certain embodiments, the expressed gene set/gene expression profile is obtained using single cell RNA-seq. In some aspects, a gene expression profile as disclosed herein can be obtained by any known or future method suitable to assess gene expression.

In accord with the foregoing, three classification methods are provided below.

### a) Classifying Cancer Epithelial Cells

Tumor cells are often very heterogeneous and are difficult to characterize with a single genotype. This heterogeneity results in different tumor cells expressing different surface receptors which may directly impact the tumor cell's ability or tendency to interact with other cells. At the same time, modern immunotherapies rely on the interaction between a tumor and a secondary cell population (e.g., immune cells) to function. Consequently, unappreciated tumor heterogeneity can have a direct impact on immunotherapy efficacy. Until now, it has been largely impossible to understand this heterogeneity in any meaningful way to determine clinical outcomes. Consequently, in a first aspect, a method is provided for classifying a tumor cell. In various aspects, tumor cells are classified to a “gene element group” which, as described further below, encompasses a set of genes identified in a gene expression profile or set of expressed genes. Specifically, the set of genes in a “gene element group” encompass genes that are upregulated relative to a baseline gene expression level (e.g., relative to a baseline gene expression level across cells across the entire tumor).

Accordingly, a method of classifying a cancer epithelial cell to a gene element group may comprise (i) obtaining a set of expressed genes in the cancer epithelial cell; (ii) determining expression levels of genes in a plurality of gene sets and ranking the expression levels in each gene set to identify a gene set having highest gene expression; and (iii) assigning the cell to a gene element group corresponding to the gene set having highest gene expression.

The gene sets having highest gene expression corresponding to each gene element group may, in general, comprise one or more (for example 10 or more, 20 or more, 30 or more, 40 or more, 50 or more, 60 or more, 70 or more, 80 or more, 90 or more, or 100 or more) genes selected from the following: A2M, AC090498-1, AC093001-1, AC105999-2, ACTA2, ACTG2, ADIRF, AFF3, AGR2, AGR3, AIF1, ALCAM, ALDH2, ALDH3B2, ALOX15B, ALOX5AP, ANAPC11, ANGPTL4, ANKRD30A, ANKRD37, ANLN, ANP32E, ANXA1, ANXA2, APOC1, APOD, APOE, AQP1, AQP3, AQP5, AR, ARC, AREG, ARFGEF3, ARL6IP1, ARMT1, ASAH1, ASCL1, ASF1B, ASPM, ATAD2, ATF3, ATP1B1, ATP5ME, AURKA, AZGP1, AZIN1, B2M, BAMBI, BATF, BGN, BIRC5, BLVRB, BMPR1B, BNIP3, BST2, BTG1, BTG2, BUB1B, C15ORF48, C1ORF162, C1orf21, C1QA, C1QB, C1QC, C6ORF15, CALD1, CALM1, CALML5, CALU, CAPG, CARD16, CAV1, CAVIN1, CAVIN3, CCDC74A, CCL20, CCL28, CCL3, CCL4, CCL5, CCN1, CCN2, CCNB1, CCNB2, CCND1, CD2, CD24, CD27, CD37, CD3D, CD3E, CD48, CD52, CD53, CD55, CD59, CD69, CD7, CD74, CD83, CD9, CD99, CDC20, CDC6, CDCA3, CDCA8, CDK1, CDK12, CDKN1A, CDKN2A, CDKN2B, CDKN3, CEACAM6, CEBPB, CEBPD, CELF2, CENPA, CENPE, CENPF, CENPK, CENPM, CENPU, CENPW, CETN2, CFD, CHI3L1, CHI3L2, CIP2A, CISD3, CKAP2, CKB, CKLF, CKS1B, CKS2, CLDN3, CLDN4, CLDN7, CLIC3, CLTC, CLU, CNN3, COL12A1, COL1A2, COL6A1, COL6A2, CORO1A, COTL1, COX6C, CP, CPB1, CRABP2, CRACR2B, CREM, CRIP1, CRIP2, CRISP3, CRYAB, CSRP1, CSRP2, CST3, CST7, CSTA, CSTB, CTD-3252C9-4, CTHRC1, CTNNB1, CTNND1, CTSC, CTSD, CTSK, CTSL, CTSS, CTSW, CTTN, CXCL1, CXCL14, CXCL17, CXCL2, CXCR4, CYB5A, CYBA, CYBB, CYC1, CYSTM1, CYTIP, DBI, DCXR, DDIT4, DEFB1, DEK, DEPP1, DHRS2, DLGAP5, DLX5, DNAJB1, DSC2, DSCAM-AS1, DSTN, DTYMK, DUSP1, DUSP2, DUT, ECT2, EDN1, EEF1B2, EFEMP1, EFHD1, EFNA1, EGR1, ELF3, ELF5, ELOC, ELOVL2, ELP2, EMP2, EMP3, ENO1, EPCAM, ERBB2, ERBB4, ESR1, EVL, FABP3, FABP5, FADS2, FAM111A, FAM111B, FAM229B, FAM3D, FASN, FBXO32, FCER1G, FDCSP, FGFBP2, FHL2, FKBP5, FN1, FOS, FOSB, FSIP1, FXYD3, FYB1, GABRP, GATA3, GBP1, GBP2, GDF15, GGH, GIMAP7, GJA1, GLYATL2, GMFG, GPR183, GPSM3, GPX1, GPX4, GRB7, GRIK1-AS1, GSN, GSTM3, GSTP1, GTSE1, GZMA, GZMK, H1-2, H1-3, H2AJ, H2AZ1, H2AZ2, H2BC11, H4C3, HCST, HDC, HELLS, HES1, HILPDA, HINT1, HLA-A, HLA-B, HLA-C, HLA-DMA, HLA-DPA1, HLA-DPB1, HLA-DQA1, HLA-DQA2, HLA-DQB1, HLA-DRA, HLA-DRB1, HLA-DRB5, HLA-E, HMGB1, HMGB2, HMGB3, HMGN2, HMMR, HNRNPH1, HSPA1A, HSPA1B, HSPA5, HSPB1, HSPE1, ICAM1, ID1, ID2, ID3, IDH2, IER2, IER3, IFI16, IFI27, IFI44L, IFI6, IFIT1, IFIT2, IFIT3, IFITM1, IFITM2, IFITM3, IGFBP3, IGFBP4, IGFBP5, IGFBP7, IGKC, IGSF6, IL2RG, IL32, IL7R, INPP4B, INTS6, IQGAP3, IRF1, ISG15, ISG20, ITGB1, ITGB2, ITGB6, ITM2A, ITM2B, JPT1, JUN, JUNB, KCNC2, KCNE4, KCNJ3, KDELR2, KIF20B, KIF23, KIF2C, KLF4, KLF6, KLK5, KLK7, KLRB1, KNL1, KPNA2, KRT10, KRT14, KRT15, KRT16, KRT17, KRT18, KRT19, KRT23, KRT5, KRT6A, KRT6B, KRT7, KRT8, KRT81, LAMB3, LAPTM4B, LAPTM5, LCK, LCN2, LDHB, LDLRAD4, LGALS1, LGALS3, LGMN, LIMD2, LMNA, LMO4, LRATD2, LSM3, LSM4, LSP1, LST1, LTB, LTF, LUM, LY6D, LY6E, LY96, LYZ, MAD2L1, MAFB, MAGED2, MAL2, MAOB, MARCKS, MARCKSL1, MDK, MEF2C, MESP1, MFAP2, MFAP5, MFGE8, MGP, MGST1, MIA, MIEN1, MIF, MKI67, MMP7, MNDA, MPC2, MRPL12, MRPL15, MRPL51, MRPS30, MRPS30-DT, MRPS34, MS4A6A, MS4A7, MSR1, MT1X, MT2A, MT-ATP8, MTCO2P12, MTDH, MT-ND4L, MUC1, MUC5B, MUCL1, MX1, MYBL2, MYBPC1, MYH9, MYL9, MYLK, MZT1, NAMPT, NCOA7, NDRG1, NDRG2, NDUFA4L2, NDUFB9, NDUFC2, NEAT1, NEK2, NFIB, NFKBIA, NFKBIZ, NKG7, NME1, NME2, NNMT, NOVA1, NPW, NR4A1, NR4A2, NUDT8, NUF2, NUPR1, NUSAP1, PAFAH1B3, PALMD, PBK, PCLAF, PCNA, PCSK1N, PDLIM4, PDZK1IP1, PEG10, PERP, PFDN2, PFN1, PFN2, PHGDH, PHGR1, PI15, PI3, PIGR, PIP, PKM, PLAAT4, PLAT, PLK1, PLS3, PMAIP1, PMEPA1, POLR2K, POSTN, PPDPF, PRC1, PRNP, PRR11, PRSS21, PRSS23, PSAP, PSCA, PSD3, PSMA7, PSMB3, PSME2, PTHLH, PTN, PTPN1, PTPRC, PTTG1, PVALB, PYDC1, RAC2, RACGAP1, RAD21, RAMP1, RAMP2, RAMP3, RAN, RANBP1, RARRES1, RBIS, RBP1, RCAN1, REEP5, REL, RGCC, RGS1, RGS10, RGS2, RHCG, RHEB, RHOB, RHOBTB3, RHOV, RNASE1, RNASEH2A, RND1, ROMO1, RP11-53019-2, RPL39L, RPS26, RRM2, RSAD2, RSU1, RUNX1, S100A1, S100A10, S100A14, S100A16, S100A2, S100A4, S100A6, S100A7, S100A8, S100A9, S100P, SAA1, SAA2, SAT1, SBSN, SCARB2, SCD, SCGB1D2, SCGB2A1, SCGB2A2, SCGB3A1, SCUBE2, SDC1, SDC2, SEC61G, SELENOP, SEMA3C, SEPTIN6, SERHL2, SERPINA1, SERPING1, SFRP1, SGK1, SH3BGRL, SH3BGRL3, SHISA2, SLC12A2, SLC18A2, SLC25A37, SLC2A3, SLC38A2, SLC39A4, SLC39A6, SLC40A1, SLC9A3R1, SLPI, SMAP2, SMC4, SMIM22, SNCG, SNRPB, SNRPG, SOCS1, SOCS3, SOD2, SOX4, SOX9, SPARC, SPARCL1, SPATS2L, SPC25, SPINT2, SPP1, SQLE, SRGN, SRP9, STARD10, STC2, STK4, STMN1, STOM, SYNM, SYTL2, TACSTD2, TAGLN, TCAF1, TCEAL4, TCIM, TFAP2B, TFDP1, TFF1, TFF3, THBS1, TIMP1, TIMP3, TK1, TM4SF1, TMC5, TMCO1, TMEM106C, TMEM123, TMEM14B, TMEM45A, TMPO, TMSB4X, TNFAIP3, TNFAIP6, TNFRSF12A, TNFSF10, TOP2A, TPI1, TPM1, TPM2, TPRG1, TPX2, TRAC, TRBC1, TRBC2, TREM2, TROAP, TRPS1, TSC22D1, TSC22D3, TSHZ2, TSPAN1, TSPAN13, TSPYL1, TTK, TTYH1, TUBA1A, TUBA1B, TUBA1C, TUBB, TUBB4B, TXNIP, TYMS, TYROBP, UBE2C, UBE2S, UBE2T, UBE2V2, UQCRQ, VASN, VEGFA, VIM, VSTM2A, VTCN1, WFDC2, WIPF1, WSB1, XBP1, XIST, YBX1, YBX3, YWHAH, YWHAZ, ZEB2, ZFAND2A, ZFP36, ZFP36L1, ZFP36L2, ZNF292, ZNF331, ZNF706, and ZWINT.

In various aspects, a gene set having highest gene expression corresponding to a gene element group, as defined below, may comprise 5 to 500 genes (e.g., 10 to 450, 20 to 400, 30 to 350, 40 to 300, 50 to 250, 60 to 200, 70 to 150, 80 to 125, or 90 to 110 genes) selected from the set of genes listed above. For example, the gene element group may comprise at least 5 genes, at least 10 genes, at least 15 genes, at least 20 genes, at least 25 genes, at least 30 genes, at least 35 genes, at least 40 genes, at least 45 genes, at least 50 genes, at least 55 genes, at least 60 genes, at least 65 genes, at least 70 genes, at least 75 genes, at least 80 genes, at least 85 genes, at least 90 genes, at least 95 genes, at least 100 genes, at least 105 genes, at least 110 genes, at least 115 genes, at least 120 genes, at least 125 genes, at least 130 genes, at least 135 genes, at least 140 genes, at least 145 genes, at least 150 genes, at least 155 genes, at least 160 genes, at least 165 genes, at least 170 genes, at least 175 genes, at least 180 genes, at least 185 genes, at least 190 genes, at least 195 genes, at least 200 genes, at least 205 genes, at least 210 genes, at least 215 genes, at least 220 genes, at least 225 genes, at least 230 genes, at least 235 genes, at least 240 genes, at least 245 genes, at least 250 genes, at least 255 genes, at least 260 genes, at least 265 genes, at least 270 genes, at least 275 genes, at least 280 genes, at least 285 genes, at least 290 genes, at least 295 genes, at least 300 genes, at least 305 genes, at least 310 genes, at least 315 genes, at least 320 genes, at least 325 genes, at least 330 genes, at least 335 genes, at least 340 genes, at least 345 genes, at least 350 genes, at least 355 genes, at least 360 genes, at least 365 genes, at least 370 genes, at least 375 genes, at least 380 genes, at least 385 genes, at least 390 genes, at least 395 genes, and at least 400 genes selected from the group provided above. In certain aspects, each gene set having highest gene expression corresponding to a gene element group comprises about 100 genes selected from the group provided above.

Certain exemplary combinations of genes that may define a set of gene element groups are shown in Table 1 below. These gene element groups each comprise about 100 genes that are uniquely overexpressed in certain cancer epithelial cells. That is, the gene set having highest gene expression comprises one or more genes from the lists provided below. In various aspects, cells may be classified to a gene element group if the gene set having highest gene expression comprises at least one gene, at least 5 genes, at least 10 genes, at least 15 genes, or at least 20 genes selected from each list provided below. Accordingly, in various aspects, cells are classified to one of ten different gene element groups as provided in Table 1 below.

TABLE 1
Gene Element Groups

| Gene | |
|---|---|
| Element | |
| Group | Overexpressed Genes |
| GE 1 | AC090498-1, AC105999-2, ADIRF, AGR2, AGR3, ALDH2, ANKRD30A, |
| | ARL6IP1, ARMT1, ATAD2, AZGP1, BATF, BMPR1B, BST2, BTG2, |
| | C15ORF48, CCDC74A, CEBPD, CFD, CLDN4, CLU, COX6C, CPB1, |
| | CRIP1, CST3, CTHRC1, CXCL14, DHRS2, DSCAM-AS1, ELF3, ELP2, |
| | ERBB4, ESR1, EVL, FABP3, FHL2, FKBP5, FSIP1, GJA1, GSTM3, HES1, |
| | HSPB1, IFI27, IFI6, IFITM1, IFITM2, IFITM3, IGFBP4, INPP4B, ISG15, |
| | JUNB, KCNE4, KCNJ3, KRT18, KRT19, LDLRAD4, MAGED2, MDK, |
| | MESP1, MGP, MGST1, MRPS30, MRPS30-DT, MS4A7, MT-ATP8, NOVA1, |
| | PEG10, PHGR1, PI15, PIP, PLAAT4, PLAT, PRSS23, PSD3, PVALB, |
| | RAMP1, RBP1, RHOBTB3, SCGB3A1, SCUBE2, SEMA3C, SERPINA1, |
| | SH3BGRL, SLC39A6, SLC40A1, SNCG, STC2, TCEAL4, TCIM, TFF1, |
| | TFF3, TIMP1, TMC5, TPM1, TPRG1, VSTM2A, VTCN1, WFDC2, XBP1, |
| | ZFP36L1 |
| GE 2 | ALDH3B2, ALOX15B, APOD, AZIN1, B2M, BNIP3, C1orf21, CALD1, CALU, |
| | CAPG, CD24, CD59, CD74, CD99, CDKN2B, CFD, CKB, CLDN3, CLDN4, |
| | CNN3, COL12A1, COX6C, CRIP1, CSRP1, CSRP2, CTNNB1, CTTN, |
| | CYSTM1, DDIT4, DHRS2, DLX5, DSC2, EFHD1, EFNA1, ELF5, ENO1, |
| | FAM229B, FASN, GJA1, GRIK1-AS1, GSTP1, H2AJ, HILPDA, HNRNPH1, |
| | HSPA5, IFI27, IFITM3, IGKC, JPT1, KCNC2, KRT15, KRT23, KRT7, |
| | LAPTM4B, LDHB, LMO4, LTF, MAFB, MAL2, MAOB, MFAP2, MGST1, |
| | MRPL15, MT1X, MUCL1, MYBPC1, NME2, NUPR1, PCSKIN, PFN2, |
| | PHGDH, PRSS23, PSMB3, PTHLH, PTPN1, RAMP1, RAMP3, RBP1, |
| | RSU1, S100A10, S100A6, SCUBE2, SFRP1, SH3BGRL, SLC39A4, |
| | SLC40A1, SOX4, STC2, STOM, TCIM, TFF3, TMSB4X, TTYH1, TUBA1A, |
| | UBE2V2, VIM, YBX1, YBX3, YWHAH, YWHAZ |
| GE 3 | A2M, ACTA2, ACTG2, ANGPTL4, ANXA1, APOD, APOE, BGN, C6ORF15, |
| | CALD1, CALML5, CAV1, CAVIN1, CAVIN3, CCL28, CCN2, CD24, CDKN2A, |
| | CHI3L1, COL1A2, COL6A1, COL6A2, COTL1, CRYAB, CSTA, CXCL2, |
| | DEFB1, DEPP1, EFEMP1, FABP5, FBXO32, FDCSP, FGFBP2, FN1, |
| | GABRP, GSTP1, HLA-A, HLA-B, ID1, IFI27, IGFBP3, IGFBP5, IGFBP7, |
| | IL32, KLK5, KLK7, KRT14, KRT15, KRT16, KRT17, KRT5, KRT6A, KRT6B, |
| | KRT81, LAMB3, LCN2, LTF, LY6D, MFAP5, MFGE8, MGP, MIA, MMP7, |
| | MT1X, MT2A, MYL9, MYLK, NDRG1, NDUFA4L2, NFKBIA, NNMT, PDLIM4, |
| | PLS3, POSTN, PRNP, PTN, RARRES1, RCAN1, RGS2, S100A2, S100A4, |
| | S100A6, S100A8, S100A9, SAA1, SAA2, SBSN, SERPING1, SFRP1, |
| | SGK1, SLC25A37, SLPI, SPARC, SPARCL1, TAGLN, THBS1, TPM2, |
| | TSHZ2, VIM, ZFP36L2 |
| GE 4 | ANLN, ANP32E, ARL6IP1, ASF1B, ASPM, ATAD2, AURKA, BIRC5, BUB1B, |
| | CCNB1, CCNB2, CDC20, CDC6, CDCA3, CDCA8, CDK1, CDKN2A, |
| | CDKN3, CENPA, CENPE, CENPF, CENPK, CENPM, CENPU, CENPW, |
| | CIP2A, CKAP2, CKLF, CKS1B, CKS2, CTHRC1, DEK, DLGAP5, DTYMK, |
| | DUT, ECT2, FAM111A, FAM111B, GGH, GTSE1, H1-2, H1-3, H2AZ1, |
| | H2AZ2, H2BC11, H4C3, HELLS, HMGB1, HMGB2, HMGB3, HMGN2, |
| | HMMR, IQGAP3, KIF20B, KIF23, KIF2C, KNL1, KPNA2, LGALS1, MAD2L1, |
| | MKI67, MT2A, MYBL2, MZT1, NEK2, NUF2, NUSAP1, PBK, PCLAF, PCNA, |
| | PLK1, PRC1, PRR11, PTTG1, RACGAP1, RAD21, RHEB, RNASEH2A, |
| | RPL39L, RRM2, SMC4, SPC25, STMN1, TFDP1, TK1, TMEM106C, TMPO, |
| | TOP2A, TPX2, TROAP, TTK, TUBA1B, TUBA1C, TUBB, TUBB4B, TYMS, |
| | UBE2C, UBE2S, UBE2T, ZWINT |
| GE 5 | AIF1, ALOX5AP, ANXA1, APOC1, APOE, AREG, C1ORF162, C1QA, |
| | C1QB, C1QC, CARD16, CCL3, CCL4, CCL5, CD2, CD27, CD37, CD3D, |
| | CD3E, CD48, CD52, CD53, CD69, CD7, CD74, CD83, CELF2, COL1A2, |
| | CORO1A, CREM, CST7, CTSL, CTSW, CXCR4, CYBB, CYTIP, DUSP2, |
| | EMP3, FCER1G, FN1, FYB1, GIMAP7, GMFG, GPR183, GPSM3, GZMA, |
| | GZMK, HCST, HLA-DPA1, HLA-DPB1, HLA-DQA1, HLA-DRA, HLA-DRB1, |
| | IGSF6, IL2RG, IL32, IL7R, ISG15, ITGB2, KLRB1, LAPTM5, LCK, LIMD2, |
| | LSP1, LST1, LTB, LY96, LYZ, MEF2C, MNDA, MS4A6A, MSR1, NKG7, |
| | PTPRC, RAC2, RGCC, RGS1, RGS2, RNASE1, S100A4, S100A6, |
| | SEPTIN6, SLC2A3, SMAP2, SOCS1, SPARC, SPP1, SRGN, STK4, |
| | TMSB4X, TNFAIP3, TRAC, TRBC1, TRBC2, TREM2, TYROBP, VIM, |
| | WIPF1, ZEB2, ZNF331 |
| GE 6 | ADIRF, ANAPC11, ATP5ME, AZGP1, BLVRB, BST2, CALM1, CCND1, CD9, |
| | CETN2, CISD3, CLDN7, COX6C, CRABP2, CRACR2B, CRIP1, CRIP2, |
| | CSTB, CYB5A, CYBA, CYC1, DBI, DCXR, DSTN, EEF1B2, ELOC, EMP2, |
| | FXYD3, GPX4, GSTM3, H2AJ, H2AZ1, HINT1, HMGB1, HSPE1, IDH2, |
| | JPT1, KDELR2, KRT10, KRT18, KRT19, KRT7, KRT8, LGALS1, LGALS3, |
| | LSM3, LSM4, LY6E, MARCKSL1, MIEN1, MIF, MPC2, MRPL12, MRPL51, |
| | MRPS34, MTDH, MUCL1, NDUFB9, NDUFC2, NME1, PAFAH1B3, PFDN2, |
| | PFN1, PIP, POLR2K, PPDPF, PSMA7, PSMB3, PSME2, RAN, RANBP1, |
| | RBIS, REEP5, ROMO1, RPS26, S100A14, S100A16, SEC61G, SELENOP, |
| | SH3BGRL, SLC9A3R1, SMIM22, SNRPB, SNRPG, SPINT2, SQLE, SRP9, |
| | STARD10, TCEAL4, TMCO1, TMEM14B, TPI1, TPM1, TSPAN13, TUBA1B, |
| | TUBB, UQCRQ, XBP1, YBX1, ZNF706 |
| GE 7 | AC093001-1, ADIRF, AGR2, AGR3, ANKRD37, APOD, AQP3, ARC, AREG, |
| | ATF3, AZGP1, BAMBI, BTG1, BTG2, C15ORF48, CALML5, CCDC74A, |
| | CCN1, CD55, CDKN1A, CEBPB, CEBPD, CFD, CLDN3, CLDN4, CST3, |
| | CTD-3252C9-4, CTSK, DHRS2, DNAJB1, DUSP1, EDN1, EGR1, ELF3, |
| | ELOVL2, ESR1, FHL2, FOS, FOSB, GATA3, GDF15, GRB7, GSTM3, H1-2, |
| | HES1, ICAM1, ID2, IER2, IER3, IFITM1, IGFBP4, IGFBP5, IRF1, JUN, |
| | JUNB, KLF4, KLF6, KRT15, KRT18, LGALS3, MAFB, MAGED2, MGP, |
| | NAMPT, NCOA7, NFKBIA, NFKBIZ, NR4A1, NR4A2, PERP, PLAT, PMAIP1, |
| | PRSS23, REL, RHOV, RND1, S100P, SAT1, SLC39A6, SLC40A1, SOCS3, |
| | SOX4, SOX9, STC2, TACSTD2, TCIM, TFF1, TIMP3, TM4SF1, |
| | TNFRSF12A, TSC22D3, TUBA1A, VASN, VEGFA, VTCN1, XBP1, |
| | ZFAND2A, ZFP36, ZFP36L1, ZFP36L2 |
| GE 8 | ADIRF, AFF3, ALCAM, ANKRD30A, ANXA2, AR, ARFGEF3, ASAH1, |
| | ATP1B1, AZGP1, BTG1, CD59, CDK12, CEBPD, CLDN3, CLDN4, CLTC, |
| | CLU, CNN3, CTNNB1, CTNND1, EFHD1, EGR1, ELF3, EPCAM, ERBB2, |
| | ESR1, EVL, FOSB, GATA3, GRB7, H4C3, HES1, HLA-B, HNRNPH1, |
| | HSPA1A, HSPA1B, IGFBP5, INTS6, ITGB1, ITGB6, ITM2B, JUN, KLF6, |
| | KRT7, LDLRAD4, LMNA, LRATD2, MAGED2, MAL2, MARCKS, MT-ND4L, |
| | MT2A, MUC1, MYH9, NEAT1, NFIB, PERP, PKM, PLAT, PMEPA1, PSAP, |
| | RAD21, RBP1, RHOB, RUNX1, S100A10, SAT1, SCARB2, SCD, SDC1, |
| | SERHL2, SH3BGRL3, SHISA2, SLC38A2, SLC39A6, SLC40A1, SOX4, |
| | SYTL2, TACSTD2, TCAF1, TCIM, TFAP2B, TIMP1, TM4SF1, TMC5, |
| | TMEM123, TPM1, TRPS1, TSC22D1, TSPYL1, TUBA1A, VEGFA, WSB1, |
| | XIST, YBX1, YBX3, ZFP36L1, ZFP36L2, ZNF292 |
| GE 9 | AC093001-1, ADIRF, AGR2, AGR3, APOD, AQP1, AQP5, AREG, ASCL1, |
| | AZGP1, BMPR1B, C15ORF48, CALML5, CCL28, CD55, CEACAM6, CFD, |
| | CLIC3, CLU, COX6C, CSTB, CTSD, CXCL14, CXCL17, DHRS2, DSCAM- |
| | AS1, DUSP1, ERBB2, FADS2, FAM3D, FHL2, GDF15, GLYATL2, GPX1, |
| | GSN, GSTP1, HDC, HSPB1, IGFBP5, ISG20, ITM2A, KRT23, KRT7, |
| | LGALS1, LGALS3, LY6E, MARCKS, MFGE8, MGP, MS4A7, MT-ATP8, |
| | MTCO2P12, MUC5B, MUCL1, NDRG2, NFKBIZ, NPW, NR4A1, NUDT8, |
| | PALMD, PDZK1IP1, PERP, PHGR1, PIP, PLAT, PRSS21, PSCA, PTHLH, |
| | PYDC1, RGS10, RGS2, RHCG, RP11-53O19-2, S100A1, S100A10, |
| | S100A6, S100A7, S100A8, S100A9, S100P, SAA2, SCGB1D2, SCGB2A1, |
| | SCGB2A2, SDC2, SERHL2, SERPINA1, SLC12A2, SLC18A2, SLPI, SYNM, |
| | TACSTD2, TFF1, TFF3, TM4SF1, TMC5, TSC22D3, TSPAN1, TXNIP, XBP1 |
| GE 10 | AGR2, APOD, AREG, AZGP1, B2M, BST2, BTG2, C15ORF48, CCL20, |
| | CD74, CEBPD, CHI3L1, CHI3L2, CP, CRISP3, CSTA, CTSC, CTSD, CTSS, |
| | CXCL1, CXCL17, CYBA, DEFB1, FDCSP, GBP1, GBP2, HLA-A, HLA-B, |
| | HLA-C, HLA-DMA, HLA-DPA1, HLA-DPB1, HLA-DQA1, HLA-DQA2, HLA- |
| | DQB1, HLA-DRA, HLA-DRB1, HLA-DRB5, HLA-E, ID3, IFI16, IFI27, IFI44L, |
| | IFI6, IFIT1, IFIT2, IFIT3, IFITM1, IFITM2, IFITM3, IGFBP7, IL32, IRF1, |
| | ISG15, KRT15, KRT19, KRT5, KRT7, LCN2, LGALS1, LGMN, LTF, LUM, |
| | LY6D, LYZ, MAFB, MARCKS, MGP, MIA, MMP7, MRPS30-DT, MX1, |
| | NNMT, PI3, PIGR, RAMP2, RARRES1, RHCG, RNASE1, RSAD2, S100A8, |
| | S100A9, S100P, SAA2, SCGB1D2, SCGB2A1, SERPING1, SLC39A6, |
| | SOD2, SPATS2L, TCIM, TFF1, TFF3, TMEM45A, TNFAIP6, TNFSF10, |
| | TXNIP, WFDC2, XBP1, ZFP36 |

Accordingly, in some aspects, the cancer epithelial cell may be assigned to a gene element 1 group (GE1) when the gene set having highest gene expression comprises at least one gene, at least 5 genes, at least 10 genes, at least 15 genes, or at least 20 genes selected from the group consisting of AC090498-1, AC105999-2, ADIRF, AGR2, AGR3, ALDH2, ANKRD30A, ARL6IP1, ARMT1, ATAD2, AZGP1, BATF, BMPR1B, BST2, BTG2, C15ORF48, CCDC74A, CEBPD, CFD, CLDN4, CLU, COX6C, CPB1, CRIP1, CST3, CTHRC1, CXCL14, DHRS2, DSCAM-AS1, ELF3, ELP2, ERBB4, ESR1, EVL, FABP3, FHL2, FKBP5, FSIP1, GJA1, GSTM3, HES1, HSPB1, IFI27, IFI6, IFITM1, IFITM2, IFITM3, IGFBP4, INPP4B, ISG15, JUNB, KCNE4, KCNJ3, KRT18, KRT19, LDLRAD4, MAGED2, MDK, MESP1, MGP, MGST1, MRPS30, MRPS30-DT, MS4A7, MT-ATP8, NOVA1, PEG10, PHGR1, PI15, PIP, PLAAT4, PLAT, PRSS23, PSD3, PVALB, RAMP1, RBP1, RHOBTB3, SCGB3A1, SCUBE2, SEMA3C, SERPINA1, SH3BGRL, SLC39A6, SLC40A1, SNCG, STC2, TCEAL4, TCIM, TFF1, TFF3, TIMP1, TMC5, TPM1, TPRG1, VSTM2A, VTCN1, WFDC2, XBP1, and ZFP36L1.

In further aspects, the cancer epithelial cell may be assigned to a gene element 2 group (GE2) when the gene set having highest gene expression comprises at least one gene, at least 5 genes, at least 10 genes, at least 15 genes, or at least 20 genes selected from the group consisting of ALDH3B2, ALOX15B, APOD, AZIN1, B2M, BNIP3, C1orf21, CALD1, CALU, CAPG, CD24, CD59, CD74, CD99, CDKN2B, CFD, CKB, CLDN3, CLDN4, CNN3, COL12A1, COX6C, CRIP1, CSRP1, CSRP2, CTNNB1, CTTN, CYSTM1, DDIT4, DHRS2, DLX5, DSC2, EFHD1, EFNA1, ELF5, ENO1, FAM229B, FASN, GJA1, GRIK1-AS1, GSTP1, H2AJ, HILPDA, HNRNPH1, HSPA5, IFI27, IFITM3, IGKC, JPT1, KCNC2, KRT15, KRT23, KRT7, LAPTM4B, LDHB, LMO4, LTF, MAFB, MAL2, MAOB, MFAP2, MGST1, MRPL15, MT1X, MUCL1, MYBPC1, NME2, NUPR1, PCSK1N, PFN2, PHGDH, PRSS23, PSMB3, PTHLH, PTPN1, RAMP1, RAMP3, RBP1, RSU1, S100A10, S100A6, SCUBE2, SFRP1, SH3BGRL, SLC39A4, SLC40A1, SOX4, STC2, STOM, TCIM, TFF3, TMSB4X, TTYH1, TUBA1A, UBE2V2, VIM, YBX1, YBX3, YWHAH, and YWHAZ.

In still further aspects, the cancer epithelial cell may be assigned to a gene element 3 group (GE3) when the gene set having highest gene expression comprises at least one gene, at least 5 genes, at least 10 genes, at least 15 genes, or at least 20 genes selected from the group consisting of A2M, ACTA2, ACTG2, ANGPTL4, ANXA1, APOD, APOE, BGN, C6ORF15, CALD1, CALML5, CAV1, CAVIN1, CAVIN3, CCL28, CCN2, CD24, CDKN2A, CHI3L1, COL1A2, COL6A1, COL6A2, COTL1, CRYAB, CSTA, CXCL2, DEFB1, DEPP1, EFEMP1, FABP5, FBXO32, FDCSP, FGFBP2, FN1, GABRP, GSTP1, HLA-A, HLA-B, ID1, IFI27, IGFBP3, IGFBP5, IGFBP7, IL32, KLK5, KLK7, KRT14, KRT15, KRT16, KRT17, KRT5, KRT6A, KRT6B, KRT81, LAMB3, LCN2, LTF, LY6D, MFAP5, MFGE8, MGP, MIA, MMP7, MT1X, MT2A, MYL9, MYLK, NDRG1, NDUFA4L2, NFKBIA, NNMT, PDLIM4, PLS3, POSTN, PRNP, PTN, RARRES1, RCAN1, RGS2, S100A2, S100A4, S100A6, S100A8, S100A9, SAA1, SAA2, SBSN, SERPING1, SFRP1, SGK1, SLC25A37, SLPI, SPARC, SPARCL1, TAGLN, THBS1, TPM2, TSHZ2, VIM, and ZFP36L2.

In various aspects, the cancer epithelial cell is assigned to a gene element 4 group (GE4) when the gene set having highest gene expression comprises at least one gene, at least 5 genes, at least 10 genes, at least 15 genes, or at least 20 genes selected from the group consisting of ANLN, ANP32E, ARL6IP1, ASF1B, ASPM, ATAD2, AURKA, BIRC5, BUB1B, CCNB1, CCNB2, CDC20, CDC6, CDCA3, CDCA8, CDK1, CDKN2A, CDKN3, CENPA, CENPE, CENPF, CENPK, CENPM, CENPU, CENPW, CIP2A, CKAP2, CKLF, CKS1B, CKS2, CTHRC1, DEK, DLGAP5, DTYMK, DUT, ECT2, FAM111A, FAM111B, GGH, GTSE1, H1-2, H1-3, H2AZ1, H2AZ2, H2BC11, H4C3, HELLS, HMGB1, HMGB2, HMGB3, HMGN2, HMMR, IQGAP3, KIF20B, KIF23, KIF2C, KNL1, KPNA2, LGALS1, MAD2L1, MKI67, MT2A, MYBL2, MZT1, NEK2, NUF2, NUSAP1, PBK, PCLAF, PCNA, PLK1, PRC1, PRR11, PTTG1, RACGAP1, RAD21, RHEB, RNASEH2A, RPL39L, RRM2, SMC4, SPC25, STMN1, TFDP1, TK1, TMEM106C, TMPO, TOP2A, TPX2, TROAP, TTK, TUBA1B, TUBA1C, TUBB, TUBB4B, TYMS, UBE2C, UBE2S, UBE2T, and ZWINT.

In various aspects, the cancer epithelial cell may be assigned to a gene element 5 group (GE5) when the gene set having highest gene expression comprises at least one gene, at least 5 genes, at least 10 genes, at least 15 genes, or at least 20 genes selected from the group consisting of AIF1, ALOX5AP, ANXA1, APOC1, APOE, AREG, C1ORF162, C1QA, C1QB, C1QC, CARD16, CCL3, CCL4, CCL5, CD2, CD27, CD37, CD3D, CD3E, CD48, CD52, CD53, CD69, CD7, CD74, CD83, CELF2, COL1A2, CORO1A, CREM, CST7, CTSL, CTSW, CXCR4, CYBB, CYTIP, DUSP2, EMP3, FCER1G, FN1, FYB1, GIMAP7, GMFG, GPR183, GPSM3, GZMA, GZMK, HCST, HLA-DPA1, HLA-DPB1, HLA-DQA1, HLA-DRA, HLA-DRB1, IGSF6, IL2RG, IL32, IL7R, ISG15, ITGB2, KLRB1, LAPTM5, LCK, LIMD2, LSP1, LST1, LTB, LY96, LYZ, MEF2C, MNDA, MS4A6A, MSR1, NKG7, PTPRC, RAC2, RGCC, RGS1, RGS2, RNASE1, S100A4, S100A6, SEPTIN6, SLC2A3, SMAP2, SOCS1, SPARC, SPP1, SRGN, STK4, TMSB4X, TNFAIP3, TRAC, TRBC1, TRBC2, TREM2, TYROBP, VIM, WIPF1, ZEB2, and ZNF331.

In various aspects, the cancer epithelial cell may be assigned to a gene element 6 group (GE6) when the gene set having highest gene expression comprises at least one gene, at least 5 genes, at least 10 genes, at least 15 genes, or at least 20 genes selected from the group consisting of ADIRF, ANAPC11, ATP5ME, AZGP1, BLVRB, BST2, CALM1, CCND1, CD9, CETN2, CISD3, CLDN7, COX6C, CRABP2, CRACR2B, CRIP1, CRIP2, CSTB, CYB5A, CYBA, CYC1, DBI, DCXR, DSTN, EEF1B2, ELOC, EMP2, FXYD3, GPX4, GSTM3, H2AJ, H2AZ1, HINT1, HMGB1, HSPE1, IDH2, JPT1, KDELR2, KRT10, KRT18, KRT19, KRT7, KRT8, LGALS1, LGALS3, LSM3, LSM4, LY6E, MARCKSL1, MIEN1, MIF, MPC2, MRPL12, MRPL51, MRPS34, MTDH, MUCL1, NDUFB9, NDUFC2, NME1, PAFAH1B3, PFDN2, PFN1, PIP, POLR2K, PPDPF, PSMA7, PSMB3, PSME2, RAN, RANBP1, RBIS, REEP5, ROMO1, RPS26, S100A14, S100A16, SEC61G, SELENOP, SH3BGRL, SLC9A3R1, SMIM22, SNRPB, SNRPG, SPINT2, SQLE, SRP9, STARD10, TCEAL4, TMCO1, TMEM14B, TPI1, TPM1, TSPAN13, TUBA1B, TUBB, UQCRQ, XBP1, YBX1, and ZNF706.

In some aspects, the cancer epithelial cell is assigned to a gene element 7 group (GE7) when the gene set having highest gene expression comprises at least one gene, at least 5 genes, at least 10 genes, at least 15 genes, or at least 20 genes selected from the group consisting of AC093001-1, ADIRF, AGR2, AGR3, ANKRD37, APOD, AQP3, ARC, AREG, ATF3, AZGP1, BAMBI, BTG1, BTG2, C15ORF48, CALML5, CCDC74A, CCN1, CD55, CDKN1A, CEBPB, CEBPD, CFD, CLDN3, CLDN4, CST3, CTD-3252C9-4, CTSK, DHRS2, DNAJB1, DUSP1, EDN1, EGR1, ELF3, ELOVL2, ESR1, FHL2, FOS, FOSB, GATA3, GDF15, GRB7, GSTM3, H1-2, HES1, ICAM1, ID2, IER2, IER3, IFITM1, IGFBP4, IGFBP5, IRF1, JUN, JUNB, KLF4, KLF6, KRT15, KRT18, LGALS3, MAFB, MAGED2, MGP, NAMPT, NCOA7, NFKBIA, NFKBIZ, NR4A1, NR4A2, PERP, PLAT, PMAIP1, PRSS23, REL, RHOV, RND1, S100P, SAT1, SLC39A6, SLC40A1, SOCS3, SOX4, SOX9, STC2, TACSTD2, TCIM, TFF1, TIMP3, TM4SF1, TNFRSF12A, TSC22D3, TUBA1A, VASN, VEGFA, VTCN1, XBP1, ZFAND2A, ZFP36, ZFP36L1, and ZFP36L2.

In still further aspects, the cancer epithelial cell is assigned to a gene element 8 group (GE8) when the gene set having highest gene expression comprises at least one gene, at least 5 genes, at least 10 genes, at least 15 genes, or at least 20 genes selected from the group consisting of ADIRF, AFF3, ALCAM, ANKRD30A, ANXA2, AR, ARFGEF3, ASAH1, ATP1B1, AZGP1, BTG1, CD59, CDK12, CEBPD, CLDN3, CLDN4, CLTC, CLU, CNN3, CTNNB1, CTNND1, EFHD1, EGR1, ELF3, EPCAM, ERBB2, ESR1, EVL, FOSB, GATA3, GRB7, H4C3, HES1, HLA-B, HNRNPH1, HSPA1A, HSPA1B, IGFBP5, INTS6, ITGB1, ITGB6, ITM2B, JUN, KLF6, KRT7, LDLRAD4, LMNA, LRATD2, MAGED2, MAL2, MARCKS, MT-ND4L, MT2A, MUC1, MYH9, NEAT1, NFIB, PERP, PKM, PLAT, PMEPA1, PSAP, RAD21, RBP1, RHOB, RUNX1, S100A10, SAT1, SCARB2, SCD, SDC1, SERHL2, SH3BGRL3, SHISA2, SLC38A2, SLC39A6, SLC40A1, SOX4, SYTL2, TACSTD2, TCAF1, TCIM, TFAP2B, TIMP1, TM4SF1, TMC5, TMEM123, TPM1, TRPS1, TSC22D1, TSPYL1, TUBA1A, VEGFA, WSB1, XIST, YBX1, YBX3, ZFP36L1, ZFP36L2, and ZNF292.

In some aspects, the cancer epithelial cell is assigned to a gene element 9 group (GE9) when the gene set having highest gene expression comprises at least one gene, at least 5 genes, at least 10 genes, at least 15 genes, or at least 20 genes selected from the group consisting of AC093001-1, ADIRF, AGR2, AGR3, APOD, AQP1, AQP5, AREG, ASCL1, AZGP1, BMPR1B, C15ORF48, CALML5, CCL28, CD55, CEACAM6, CFD, CLIC3, CLU, COX6C, CSTB, CTSD, CXCL14, CXCL17, DHRS2, DSCAM-AS1, DUSP1, ERBB2, FADS2, FAM3D, FHL2, GDF15, GLYATL2, GPX1, GSN, GSTP1, HDC, HSPB1, IGFBP5, ISG20, ITM2A, KRT23, KRT7, LGALS1, LGALS3, LY6E, MARCKS, MFGE8, MGP, MS4A7, MT-ATP8, MTCO2P12, MUC5B, MUCL1, NDRG2, NFKBIZ, NPW, NR4A1, NUDT8, PALMD, PDZK1IP1, PERP, PHGR1, PIP, PLAT, PRSS21, PSCA, PTHLH, PYDC1, RGS10, RGS2, RHCG, RP11-53019-2, S100A1, S100A10, S100A6, S100A7, S100A8, S100A9, S100P, SAA2, SCGB1D2, SCGB2A1, SCGB2A2, SDC2, SERHL2, SERPINA1, SLC12A2, SLC18A2, SLPI, SYNM, TACSTD2, TFF1, TFF3, TM4SF1, TMC5, TSC22D3, TSPAN1, TXNIP, and XBP1.

In some aspects, the cancer epithelial cell is assigned to a gene element 10 group (GE10) when the gene set having highest gene expression comprises at least one gene, at least 5 genes, at least 10 genes, at least 15 genes, or at least 20 genes selected from the group consisting of AGR2, APOD, AREG, AZGP1, B2M, BST2, BTG2, C15ORF48, CCL20, CD74, CEBPD, CHI3L1, CHI3L2, CP, CRISP3, CSTA, CTSC, CTSD, CTSS, CXCL1, CXCL17, CYBA, DEFB1, FDCSP, GBP1, GBP2, HLA-A, HLA-B, HLA-C, HLA-DMA, HLA-DPA1, HLA-DPB1, HLA-DQA1, HLA-DQA2, HLA-DQB1, HLA-DRA, HLA-DRB1, HLA-DRB5, HLA-E, ID3, IFI16, IFI27, IFI44L, IFI6, IFIT1, IFIT2, IFIT3, IFITM1, IFITM2, IFITM3, IGFBP7, IL32, IRF1, ISG15, KRT15, KRT19, KRT5, KRT7, LCN2, LGALS1, LGMN, LTF, LUM, LY6D, LYZ, MAFB, MARCKS, MGP, MIA, MMP7, MRPS30-DT, MX1, NNMT, PI3, PIGR, RAMP2, RARRES1, RHCG, RNASE1, RSAD2, S100A8, S100A9, S100P, SAA2, SCGB1D2, SCGB2A1, SERPING1, SLC39A6, SOD2, SPATS2L, TCIM, TFF1, TFF3, TMEM45A, TNFAIP6, TNFSF10, TXNIP, WFDC2, XBP1, and ZFP36.

As mentioned, the gene expression profile may be determined using any standard method in the art. For example, the gene expression profile may be obtained from one or more cells in a tumor sample using RNA-seq (e.g., single-cell RNA seq).

In further aspects, more than one tumor cells may be classified according to the methods provided above. This allows for a determination of intra-tumoral transcriptional heterogeneity (ITTH) which, as described below, allows for a granular and more informative view of a tumor's potential responsiveness to treatment. Accordingly, in various aspects, a method of determining a level of intra-tumoral transcriptional heterogeneity (ITTH) in a tumor is provided, the method comprising classifying a plurality of cancer epithelial cells in the tumor according to the method provided herein.

### (b) Classifying Secondary Cell Populations

In various aspects, methods are also provided for classifying a secondary cell. As used herein, the term “secondary cell” refers to a cell or a group of cells that interact with a tumor in vivo. For example, a secondary cell may be an immune cell, an epithelial cell, a nervous cell, an arterial or venous cell, or any cell that directly or indirectly contacts, communicates, or signals a tumor cell. In various aspects a secondary cell may be an immune cell (e.g., a cytotoxic T cell, a natural killer (NK) cell, CD8 T cells, B cells, or myeloid cells). In certain aspects, the secondary cell is a natural killer cell and the methods provided herein allow for the classification of NK cells.

In various aspects, a method of classifying a natural killer cell (NK cell) to an NK cell subset is provided, the method comprising: (i) obtaining a set of expressed genes in the natural killer cell, (ii) determining expression levels of genes in a plurality of gene sets and ranking the expression levels in each gene set to identify a gene set having highest gene expression, (iii) assigning the NK cell to an NK cell subset corresponding to the gene set having highest gene expression.

The gene sets corresponding to each NK cell subset may, in general, comprise one or more genes selected from the following: ABCA1, ALOX12, CALD1, CAVIN2, CCL4, CCL5, CD74, CLU, CMKLR1, CR2, CX3CR1, CXCF1, DTX1, DUSP1, DUSP2, ETS1, F5, FAM81A, FCGR3A, FGFBP2, FOS, FOSB, GAS2L1, GFRA2, GP6, GZMA, GZMB, GZMH, GZMK, HEATR9, HES1, HLA-DRB1, HSPE1, IFI44L, IFI6, IFIT3, IL7R, ISG15, ITGAX, JUN, KLRC1, KLRG1, LTB, LTBP1, MID1, MPIG6B, MYADM, NHSL2, NR4A1, NR4A2, NR4A3, NYLK, PARVB, PLXNA4, PRF1, RASGRP2, RHPN1, S1PR5, SCD, SELL, SLC6A4, SLC7A5, SPON2, THBS1, TMTC1, TNFAIP3, TUBB1, VWF, and XDH.

The gene set corresponding to each NK cell subset may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, and 45 or more genes from the list provided above. In some aspects, a gene set corresponding to each NK cell subset may comprise 3 to 5 genes (e.g., 3, 4, or 5) genes. In some aspects, a gene set corresponding to each NK cell subset may comprise 5 to 10 genes (e.g., 5, 6, 7, 8, 9, or 10) genes selected from the group listed above. In some aspects, a gene set corresponding to each NK cell subset may comprise 10-15 genes (e.g., 10, 11, 12, 13, 14, or 15) genes selected from the group listed above. In some aspects, a gene set corresponding to each NK cell subset may comprise 15-20 genes (e.g., 15, 16, 17, 18, 19, or 20) genes selected from the group listed above. In further aspects, a gene set corresponding to each NK cell subset may comprise 20 to 25 genes (e.g., 20, 21, 22, 23, 24, or 25) from the group listed above. In still further aspects, the gene set corresponding to each NK cell subset may comprise 25 to 30 genes (e.g., 25, 26, 27, 28, 29 or 30) selected from the group listed above. In still further aspects, the gene set corresponding to each NK cell subset may comprise 30 to 35 genes (e.g., 30, 31, 32, 33, 34 or 35) selected from the group listed above. In still further aspects, the gene set corresponding to each NK cell subset may comprise 35 to 40 genes (e.g., 35, 36, 37, 38, 39, or 40) selected from the group listed above. In still further aspects, the gene set corresponding to each NK cell subset may comprise 40 to 45 genes (e.g., 40, 41, 42, 43, 44, or 45) selected from the group listed above.

In certain embodiments, an NK cell subset may be defined according to one of the following gene sets in Table 2.

TABLE 2
NK Subsets

| NK Cell | |
|---|---|
| Subset | Gene Set |
| NK-0 | FCGR3A, PRF1, FGFBP2, GZMH, ETS1 |
| NK-1 | NR4A1, NR4A2, DUSP1, DUSP2, FOS, JUN |
| NK-2 | FCGR3A, PRF1, FGFBP2, GZMA, GZMB, CXCF1, |
| | SPON2, CX3CR1, S1PR5 |
| NK-3 | GZMK, SELL, IL7R, LTB |
| NK-4 | ISG15, IFI6, IFIT3, IFI44L |
| NK-5 | CCL5, HLA-DRB1, KLRC1, CD74, MYADM, HSPE1 |
| rNK | ABCA1, ALOX12, CALD1, CAVIN2, CCL4, CLU, CMKLR1, |
| cell | CR2, CX3CR1, DTX1, DUSP1, F5, FAM81A, FOS, FOSB, |
| | GAS2L1, GFRA2, GP6, HEATR9, HES1, ITGAX, JUN, |
| | KLRG1, LTBP1, MID1, MPIG6B, NHSL2, NR4A1, NR4A2, |
| | NR4A3, NYLK, PARVB, PLXNA4, RASGRP2, RHPN1, SCD, |
| | SLC6A4, SLC7A5, THBS1, TMTC1, TNFAIP3, TUBB1, |
| | VWF, XDH |

In some aspects, an NK cell is assigned to NK subset NK-0 when the gene set having highest gene expression comprises at least one gene, at least 1, at least 2, at least 3, at least 4, or 5 genes selected from the group consisting of FCGR3A, PRF1, FGFBP2, GZMH, and ETS1. In various aspects, an NK cell is assigned to NK subset NK-0 when the gene set having highest gene expression comprises FCGR3A, PRF1, FGFBP2, GZMH, and ETS1.

In some aspects, an NK cell is assigned to NK subset NK-1 when the gene set having highest gene expression comprises at least one gene, at least 1, at least 2, at least 3, at least 4, at least 5, or 6 genes selected from the group consisting of NR4A1, NR4A2, DUSP1, DUSP2, FOS, JUN. In various aspects, an NK cell is assigned to NK subset NK-1 when the gene set having highest gene expression comprises NR4A1, NR4A2, DUSP1, DUSP2, FOS, and JUN. In some aspects, an NK cell is assigned to NK subset NK-2 when the gene set having highest gene expression comprises at least one gene, at least 1, at least 2, at least 3, at least 4, at least 5, at least 6, at least 7, at least 8, or 9 genes selected from the group consisting of FCGR3A, PRF1, FGFBP2, GZMA, GZMB, CXCF1, SPON2, CX3CR1, S1PR5. In various aspects, an NK cell is assigned to NK subset NK-2 when the gene set having highest gene expression comprises FCGR3A, PRF1, FGFBP2, GZMA, GZMB, CXCF1, SPON2. CX3CR1, and S1PR5.

In some aspects, an NK cell is assigned to NK subset NK-3 when the gene set having highest gene expression comprises at least one gene, at least 1, at least 2, at least 3, or 4 genes selected from the group consisting of GZMK, SELL, IL7R, LTB. In various aspects, an NK cell is assigned to NK subset NK-3 when the gene set having highest gene expression comprises GZMK, SELL, IL7R, and LTB.

In some aspects, an NK cell is assigned to NK subset NK-4 when the gene set having highest gene expression comprises at least one gene, at least 1, at least 2, at least 3, or 4 genes selected from the group consisting of ISG15, IFI6, IFIT3, and IFI44L. In various aspects, an NK cell is assigned to NK subset NK-4 when the gene set having highest gene expression comprises ISG15, IFI6, IFIT3, and IFI44L.

In some aspects, an NK cell is assigned to NK subset NK-5 when the gene set having highest gene expression comprises at least one gene, at least 1, at least 2, at least 3, at least 4, at least 5, or 6 genes selected from the group consisting of CCL5, HLA-DRB1, KLRC1, CD74. MYADM, and HSPE1. In various aspects, an NK cell is assigned to NK subset NK-5 when the gene set having highest gene expression comprises CCL5, HLA-DRB1, KLRC1, CD74, MYADM, and HSPE1.

In some aspects, an NK cell is assigned to NK subset rNK when the gene set having highest gene expression comprises at least one gene, at least 1, at least 2, at least 3, at least 4, at least 5, at least 6, at least 7, at least 8, at least 9, at least 10, at least 11, or at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, or at least 42 genes selected from the group consisting of ABCA1, ALOX12, CALD1, CAVIN2, CCL4, CLU, CMKLR1, CR2, CX3CR1, DTX1, DUSP1, F5, FAM81A, FOS, FOSB, GAS2L1, GFRA2, GP6, HEATR9, HES1, ITGAX, JUN, KLRG1, LTBP1, MID1, MPIG6B, NHSL2, NR4A1, NR4A2, NR4A3, NYLK, PARVB, PLXNA4, RASGRP2, RHPN1, SCD, SLC6A4, SLC7A5, THBS1, TMTC1, TNFAIP3, TUBB1, VWF, XDH. In various aspects, an NK cell is assigned to NK subset rNK when the gene set having highest gene expression comprises ABCA1, ALOX12, CALD1, CAVIN2, CCL4, CLU, CMKLR1, CR2, CX3CR1, DTX1, DUSP1, F5, FAM81A, FOS, FOSB, GAS2L1, GFRA2, GP6, HEATR9, HES1, ITGAX, JUN, KLRG1, LTBP1, MID1, MPIG6B, NHSL2, NR4A1, NR4A2, NR4A3, NYLK, PARVB, PLXNA4, RASGRP2, RHPN1, SCD, SLC6A4, SLC7A5, THBS1, TMTC1, TNFAIP3, TUBB1, VWF, and XDH.

Each NK cell subset defined above provides additional information about the function and role of cells classified in it. For example, in various aspects, NK-0 and NK-2 express high levels of FCGR3A (CD16) and cytolytic molecules (granzymes and PRF1), which suggests they are similar to CD56dim NK. NK-0 is enriched for KLRC2, ETS1, and effector genes (GZMH, CCL5), which closely resembles gene expression profiles previously described for ‘memory-like’ NK cells. NK-2 is defined by increased expression of cytotoxicity-related genes (GZMA, GZMB, PRF1, SPON2) and S1PR5, which has been previously described in CD56dim bone marrow NK cells. NK-4 is predominated by genes involved in interferon signaling suggesting that this subset may be influenced by interferon-high tumor microenvironments and consists of activated NK cells involved in the direct anti-tumor response. NK-3 cells appear to have features of tissue-resident NK cells, with upregulated expression of SELL, IL7R, and GZMK, as well as reduced expression of cytolytic genes and FCGR3A (CD16). In contrast, genes of inactivity and reduced cytotoxicity were upregulated in clusters NK-1 and NK-5. NK-1 most notably was marked by genes related to the NR4A family, JUN, FOS, and DUSP1. NR4A are a family of orphan nuclear receptors which act as transcription factors; they are thought to negatively regulate T cell cytotoxicity and have been described as marking specific NK cells with reduced interferon gamma production. NK-5 had reduced expression of cytolytic genes and FCGR3A (CD16) and increased expression of KLRC1 and CD96, which are inactivators of NK cell activity.

In various aspects, rNK subset cells refer to reprogrammed NK cells which as their name suggest, are NK cells reprogrammed following exposure to cancer cells. These cells have been altered by the tumor microenvironment or by cancer cells directly so that they now promote cancer cell growth, progression and/or metastasis. These cells were found to have a different expression pattern for NR4A family, which are a family of orphan nuclear receptors that act as transcription factors. As they negatively regulate T cell cytotoxicity they have been described as marking specific NK cells that have reduced interferon gamma. Consequently, these cells are of special interest as targets for immunotherapy. In certain aspects, rNK subset cells are considered a “secondary cell population” in further methods below.

### (c) InteractPrint-Associating Tumor Cell and Secondary Cell Classifications

Further aspects of the present disclosure are directed to methods of determining a level of interaction between a tumor and a secondary cell population. As used herein “level of interaction” refers to a degree to which the secondary cell population supports or promotes growth of the tumor or a tumor cell. Certain gene element groups, defined above, are characterized as “activating” which means cells classified into these groups show an increased level of interaction with a secondary cell population and consequently may be more susceptible to a therapeutic that targets that secondary cell population. Conversely, gene element groups characterized as “inactivating” generally show less of an interaction with the secondary cell population and may be less susceptible to a therapeutic that targets that secondary cell population. The methods herein provide a way to evaluate a tumor's level of interaction with a given secondary cell population, by determining the percentage of “activating” vs. “inactivating” cells in the tumor. This information may then be used to predict the tumor's susceptibility to a given therapeutic.

In various aspects, a method of determining a level of interaction between a tumor and a secondary cell population is provided, the method comprising: (a) obtaining a population of cancer epithelial cells from the tumor; (b) assigning each cancer epithelial cell obtained in (a) to a gene element group as described previously, (c) determining average expression of each gene element group in (b) across the population of cancer epithelial cells); (d) obtaining a set of prioritized receptor-ligand pairs across the tumor and the secondary cell population, each comprising a ligand expressed by a cancer epithelial cell assigned in step (b) and a prioritized receptor expressed by a secondary cell; (e) determining average expression of prioritized receptors from the set of prioritized receptor-ligand pairs in the secondary cell population; and (f) determining the level of interaction between the tumor and the secondary cell population based on the average expression of each gene element group in (c) and the average expression of prioritized receptors in (e).

In various aspects, the prioritized receptor-ligand pairs in (d) can increase or decrease an interaction between the cancer epithelial cell and the secondary cell. In other words, the prioritized receptor-ligand pairs are associated with either “activating” or “inactivating’ gene element groups such that cells classified as “activating” express ligands from prioritized receptor-ligand pairs that increase the level of interaction between the cancer epithelial cell and the secondary cells and cells classified as “inactivating” express ligands from prioritized receptor-ligand pairs that decrease the level of interaction between the cancer epithelial cell and the secondary cell.

It is evident that whether a gene element group and a prioritized receptor-ligand pair is considered “activating” or “inactivating” depends largely on the identity of the secondary cell population. Accordingly, while the identity of each possible gene element group remains constant (e.g., is determined by relative expression of the genes listed in Table 1 above), whether the gene element group is “activating” or “inactivating” is not known until a secondary cell population is selected.

In various methods herein, the secondary cell population comprises an immune cell (e.g., CD8+ T cells, NK cells, B cells, or myeloid cells), fibroblasts, or endothelial cells. For example, the secondary cell population may comprise a population of rNK cells (reprogrammed NK cells) identified using the methods described above. When, for example, the secondary population comprises rNK cells, the activating gene element groups may include GE2, GE3, GE4, GE5, GE10 and/or GE11 and the inactivating gene element groups may include GE1, GE6, GE7, GE8, and/or GE9.

In further aspects, the level of interaction in step (f) is positively weighted by average expression of gene element group classified as “activating” and negatively weighted by average expression of gene element groups classified as “inactivating”. In further aspects, (f) is calculated using an equation comprising:

$$IP = {\sum }_{i = 1}^{10} ( {e}_{i} ) ( {R}_{i} ) ( w )$$

wherein i corresponds to each gene element group, eᵢ is average expression of each gene element group, Rᵢ is the number of prioritized receptors on the secondary cell type and w is positive 1 for an activating gene element group and negative 1 for an inactivating gene element group.’

In various aspects, the level of interaction in (f) is further based on average levels of one or more interacting factors associated an interaction between the tumor cell and secondary cell population. These interacting factors are generally secreted or expressed factors that act directly or indirectly on the tumor cell, secondary cell or both the tumor cell and secondary cell to either increase or decrease the level of their interaction. In some aspects, the interacting factors may be autocrine factors (e.g., factors secreted by the tumor cell or secondary cell population), paracrine factors (e.g. factors secreted by a neighboring cell in or near the tumor microenvironment), a juxtacrine factor (e.g., a factor secreted by a cell in direct connection to the tumor cell or secondary cell) or an endocrine factor (e.g., a factor secreted elsewhere in the organism that directly or indirectly influences the level of interaction between the tumor cell and secondary cell population. Exemplary interacting factors can include, but are not limited to, cytokines, chemokines, extracellular matrix remodeling factors (MMP), secreted peptides, hormones, neuromodulators, growth factors, metabolic factors, or a combination thereof. In some aspects, the level of interaction in (f) is based on average levels of more than one interacting factors as described herein.

In various aspects, the effect of these levels of the interacting factors are incorporated into the overall level of interaction between the two cell populations in a similar way to the effect of prioritized receptor-ligand pairs as described above. Specifically, depending on the identity of the “interacting factor”, just as above for the “prioritized receptor-ligand pairs”, the GE cell classes can be sorted into “activating” or “inactivating” depending on whether the “interacting factor” increases or decreases the level of interaction between a cancer cell and the secondary cell population of interest. For example, an “interacting factor” like a cytokine or chemokine that promotes interaction between a tumor cell and a neighboring immune cell would correspond to an “activating” GE class. Accordingly, each GE class is classified as “inactivating” or “deactivating” depending on whether they are or are not influenced by the interacting factor. Once the overall level of the “interacting factor” is determined for each cancer cell-secondary cell pairing, the overall level of interaction can be determined by an equation modified to the one provided above: IP=IP(Rᵢ)+IP(B1ᵢ)+IP(B2ᵢ), where

$$IP ( {R}_{i} ) is {\sum }_{i = 1}^{10} ( {e}_{i} ) ( {R}_{i} ) ( w )$$

as described above and each IP(Bᵢ) (where B can be B1, B2, B3, etc)), is defined by the following equation

$${\sum }_{i = 1}^{10} ( {e}_{i} ) ( {B}_{i} ) ( {w}_{Bi} ) .$$

where i corresponds to each gene element group, eᵢ is average expression of each gene element group (as described above), and Bᵢ is the average level of an interacting factor associated with the secondary cell population and w_{Bi} is positive 1 for an activating gene element group and negative 1 for an inactivating gene element group. Therefore, it is possible to continually refine the level of interaction between a given tumor cell population and a secondary cell population by adding on additional weighted “interaction scores” corresponding to each additional “interacting factor” that might influence the interaction between the two cell populations.

In accord with the foregoing, the level of interaction between two cell populations (e.g., a tumor cell population and a secondary cell population) may be based on: (a) levels of receptor-ligand pairs expressed by the tumor cell and secondary cell, (b) levels of one or more “interacting factors” acting on the tumor cell and/or secondary cell, or a combination of (a) and (b). Each GE class is expected to interact differently with a given secondary cell population depending on the identity of each receptor-ligand pair and the interacting factor and the overall method provided herein, is capable of capturing this nuance.

As described further below, tumors having a high “level of interaction” with a secondary population (e.g., having an IP value greater than a certain threshold) would be expected to have increased susceptibility to a therapy that targets that secondary cell population and/or targets the interaction between the tumor and the secondary cell. Likewise, tumors having a low “level of interaction” with a secondary cell population (e.g., having an IP value lower than a certain threshold) would be expected to have less susceptibility to a therapy that targets that secondary cell population and/or targets the interaction between the tumor and the secondary cell. Accordingly, the measure of “interaction” using the methods described herein may better inform tumor therapeutics, as described in more detail below.

### III. METHODS OF TREATING CANCER IN A SUBJECT

In accordance with aspects of the present disclosure, a method of treating cancer (e.g., a tumor) in a subject are provided. In certain aspects, methods of treating cancer (e.g., treating a tumor) in a subject in need thereof comprise administering an immunotherapy to the subject. Methods are also provided for determining whether a subject is a suitable candidate for an immunotherapy.

As used herein, a suitable subject includes a mammal, a human, a livestock animal, a companion animal, a lab animal, or a zoological animal. In some embodiments, a subject may be a rodent, e.g., a mouse, a rat, a guinea pig, etc. In other embodiments, a subject may be a livestock animal. Non-limiting examples of suitable livestock animals may include pigs, cows, horses, goats, sheep, llamas and alpacas. In yet other embodiments, a subject may be a companion animal. Non-limiting examples of companion animals may include pets such as dogs (canines), cats (felines), rabbits, and birds. In yet other embodiments, a subject may be a zoological animal. As used herein, a “zoological animal” refers to an animal that may be found in a zoo. Such animals may include non-human primates, large cats, wolves, and bears. In other embodiments, the animal is a laboratory animal. Non-limiting examples of a laboratory animal may include rodents, canines, felines, and non-human primates. In some embodiments, the animal is a rodent. Non-limiting examples of rodents may include mice, rats, guinea pigs, etc. In preferred embodiments, the subject is a human. In other preferred embodiments, the subject is a canine.

In various aspects, a subject may be evaluated for candidacy for an immunotherapy by determining the level of interaction between the tumor and a secondary cell, using InteractPrint as described above, and then determining that the subject is a candidate for immunotherapy if the level of interaction exceeds a threshold. In various aspects, the secondary cell may be directly or indirectly targeted by the immunotherapy (for example, the secondary cell might express a receptor or protein that is modulated by an active agent in the immunotherapy), or the secondary cell may interact with a cell that is directly or indirectly targeted by the immunotherapy. It should be appreciated that the term “directly or indirectly targeted” is to be interpreted broadly and is meant to encompass any mechanism of action of a given immunotherapy. Nevertheless, in some non-limiting aspects, the secondary cell expresses a receptor that is targeted by the immunotherapy (for example, a secondary cell that expresses a PD-1 receptor that is the target of an anti-PD-1 immunotherapy).

In various aspects, a method of developing a new immunotherapy is provided. In aspects, this method may comprise (a) obtaining one or more candidate cell sets for targeting with a potential immunotherapy, where each candidate cell set comprises a population of tumor cells and a population of secondary cells that interact with or are suspected of interacting with the tumor cells, (b) determining a level of interaction between the tumor cells and the secondary cells in each candidate set according to the methods provided herein, and (c) selecting a cell set having a level of interaction that exceeds a threshold for further development of an immunotherapy that alters or exploits the level of interaction between the cell populations in the selected set. In various aspects, the secondary cells in each candidate cell set may comprise immune cells. For example, different sets of immune cells (e.g., T cells, macrophages, NK cells, or others) may be evaluated for their level of interaction with a given tumor cell population to identify the best candidate for targeting with a new immunotherapy strategy. In various aspects, the immunotherapy is developed to increase activity of the immune cell population in the selected cell set and/or reduce immune suppression by the tumor cells in the selected cell set.

In further aspects, a method of treating a cancer is provided, the method comprising administering an immunotherapy to a subject, wherein the subject has been identified as a good candidate for the immunotherapy according to the disclosure herein. In some aspects, the method comprises administering a new immunotherapy developed using the methods herein to the subject.

As used herein, “immunotherapy” refers to a therapy that activates or augments immune cells or reduces the overall immune suppression of the tumor microenvironment. Immunotherapy may comprise, for example, use of cancer vaccines and/or sensitized antigen presenting cells. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can be a cell-based therapy and involve delivery of genetically engineered cells that target the cancer or otherwise modulate the immune response to the cancer. For example, some cell-based immunotherapies include CAR-T cells or CAR-NK cells and comprise engineered T-cells or NK-cells expressing chimeric antigen receptors (CAR) that are designed to target specific receptors or proteins on tumor cells and then induce tumor cell lysis or killing by the engineered cell. Immunotherapy can also focus on using the cytotoxic lymphocyte-recognized epitopes of cancer cell lines. In some aspects, the immunotherapy may comprise a T-cell directed therapy. For example, in some aspects, the immunotherapy may comprise an immune checkpoint inhibitor (ICI). In some aspects, the ICI compounds comprise one or more ICI compounds disclosed herein. In some aspects, ICI compound can comprise an inhibitor of PD-1, PD-L1, TIM-3, LAG-3, CTLA-4, CSF-1R, or any combination thereof. In some aspects, ICI can comprise of CTLA-4 blocking antibodies (Ipilimumab (Yervoy) and tremelimumab (Imjuno)), PD-1 inhibitors (Pembrolizumab (Keytruda), Nivolumab (Opdivo), Cemiplimab (Libtayo), CT-011 (Pidilizumab), AMP224), PD-L1 inhibitors (Atezolizumab (tecentriq), Avelumab (Bavencio), Durvalumab (Imfinzi), BMS-936559), Lag3 inhibitors (Relatlimab), combination of Lag3 and PD1 inhibitor (PD-1 inhibitor nivolumab (Opdualag) OX40 inhibitor (MEDI6469), CD160 inhibitor (BY55) and/or CSF-1R inhibitor (PLX3397, PLX486, RG7155, AMG820, ARRY-382, FPA008, IMC-CS4, JNJ-40346527, MCS 110). In various aspects, the ICI compound comprises an inhibitor of PD-1 or PD-L1.

In certain aspects, the present disclosure provides for use of one or more anticancer therapies in combination with the immunotherapy (e.g., ICI) described above. Non-limiting examples of non-ICI therapy comprise chemotherapy, anti-mitotic compounds, surgery, radiation, hormone therapy, angiogenesis inhibitors, and/or stem cell transplantation. In accordance with some embodiments of the disclosure, the therapies that may be prescribed to a subject with increased likelihood of cancer metastases may be selected, used and/or administered to treat a cancer, a solid tumor, a metastasis, or any combination thereof.

In some embodiments, one or more anticancer therapies may be chemotherapy. Chemotherapeutic agents may be selected from any one or more of cytotoxic antibiotics, antimetabolities, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: treosulfan, and trofosfamide; plant alkaloids: vinblastine, paclitaxel, docetaxol; DNA topoisomerase inhibitors: doxorubicin, epirubicin, etoposide, camptothecin, topotecan, irinotecan, teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well known in the art (e.g., Olaparib, ABT-888, BSI-201, BGP-15, INO-1001, PJ34, 3-aminobenzamide, 4-amino-1,8-naphthalimide, 6(5H)-phenanthridinone, benzamide, NU1025).

In some embodiments, one or more anticancer therapies may be radiation therapy. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. In some aspects, the radiation therapy can be administered as external beam radiation or teletherapy wherein the radiation is directed from a remote source. In other aspects, the radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In some embodiments, one or more anticancer therapies may be hormonal therapy, Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In certain embodiments, the duration and/or dose of treatment with anticancer therapies may vary according to the particular anti-cancer agent or combination thereof. An appropriate treatment time for a particular cancer therapeutic agent will be appreciated by the skilled artisan. In some embodiments, the continued assessment of optimal treatment schedules for each cancer therapeutic agent is contemplated, where the genetic signature of the cancer of the subject as determined by the methods of the disclosure is a factor in determining optimal treatment doses and schedules.

In some embodiments, methods of treatment disclosed herein can impair tumor growth progression compared to tumor growth in an untreated subject with identical disease condition and predicted outcome. In some embodiments, tumor growth can be stopped following treatments according to the methods disclosed herein. In other embodiments, tumor growth can be impaired at least about 5% or greater to at least about 100%, at least about 10% or greater to at least about 95% or greater, at least about 20% or greater to at least about 80% or greater, at least about 40% or greater to at least about 60% or greater compared to an untreated subject with identical disease condition and predicted outcome. In other words, tumors in subject treated according to the methods disclosed herein grow at least 5% less (or more as described above) when compared to an untreated subject with identical disease condition and predicted outcome. In some embodiments, tumor growth can be impaired at least about 5% or greater, at least about 10% or greater, at least about 15% or greater, at least about 20% or greater, at least about 25% or greater, at least about 30% or greater, at least about 35% or greater, at least about 40% or greater, at least about 45% or greater, at least about 50% or greater, at least about 55% or greater, at least about 60% or greater, at least about 65% or greater, at least about 70% or greater, at least about 75% or greater, at least about 80% or greater, at least about 85% or greater, at least about 90% or greater, at least about 95% or greater, at least about 100% compared to an untreated subject with identical disease condition and predicted outcome. In some embodiments, tumor growth can be impaired at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% compared to an untreated subject with identical disease condition and predicted outcome.

In some embodiments, treatment of a tumor according to the methods disclosed herein can result in a shrinking of the tumor in comparison to the starting size of the tumor. In some embodiments, tumor shrinking may be at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% (meaning that the tumor is completely gone after treatment) compared to the starting size of the tumor.

In various embodiments, treatments administered according to the methods disclosed herein can improve patient life expectancy compared to the life expectancy of an untreated subject with identical disease condition (e.g., tumor presence) and predicted outcome. As used herein, “patient life expectancy” is defined as the time at which 50 percent of subjects are alive and 50 percent have passed away. In some embodiments, patient life expectancy can be indefinite following treatment according to the methods disclosed herein. In other aspects, patient life expectancy can be increased at least about 5% or greater to at least about 100%, at least about 10% or greater to at least about 95% or greater, at least about 20% or greater to at least about 80% or greater, at least about 40% or greater to at least about 60% or greater compared to an untreated subject with identical disease condition and predicted outcome. In some embodiments, patient life expectancy can be increased at least about 5% or greater, at least about 10% or greater, at least about 15% or greater, at least about 20% or greater, at least about 25% or greater, at least about 30% or greater, at least about 35% or greater, at least about 40% or greater, at least about 45% or greater, at least about 50% or greater, at least about 55% or greater, at least about 60% or greater, at least about 65% or greater, at least about 70% or greater, at least about 75% or greater, at least about 80% or greater, at least about 85% or greater, at least about 90% or greater, at least about 95% or greater, at least about 100% compared to an untreated subject with identical disease condition and predicted outcome. In some embodiments, patient life expectancy can be increased at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% compared to an untreated patient with identical disease condition and predicted outcome

The methods and disclosure above provide for various methods of analyzing and characterizing a specific tumor microenvironment in a subject. As described above, analyzing the tumor microenvironment can lead to many different applications. For instance, a subject may be identified as a suitable (or not suitable) candidate for a given immunotherapy (e.g., the method can be used as a “companion diagnostic” (CDx) or “complementary diagnostic” for an immunotherapy of interest). Likewise, characterizing the tumor microenvironment can be applied to tailor clinical trial patient selection to improve clinical trial outcomes and reduce costs. Use of the disclosed methods in “companion diagnostic” applications can further assist in approval of reimbursements of specific therapies in certain patient populations. These applications are not meant to be limiting and other applications may be envisioned by those of skill in the art.

### V. KITS

The present disclosure provides kits for performing any of the methods disclosed herein. In some aspects, the present disclosure provides a kit for determining a gene expression in a plurality of cells isolated from a tumor. Such a kit may comprise a means for determining expression level of any combination of genes that make up any of the gene element groups or NK cell subsets as disclosed herein.

Any of the kits disclosed herein may further comprise a container for placing a biological sample, and optionally a tool for collecting a biological sample from a subject. Alternatively, or in addition, the kit may further comprise one or more reagents for determining gene expression levels of the one or more genes in any of the gene element groups or NK cell subset groups as disclosed herein from the biological sample. In certain aspects, the biological sample comprises more than one tumor cell obtained from a subject and the kit provides reagents for detecting gene expression in each cell isolated from the sample. For example, in some aspects, the kit provides reagents for single cell RNA-seq (e.g., primers, nucleotides, markers, buffers) to measure gene expression of one or more genes listed above in an individual cell.

Any of the kits may further comprise an instruction manual providing guidance for using the kit to determine a gene expression panel having any combination of the one or more genes of the gene element groups or NK gene subsets as disclosed herein.

Further, any of the kits disclosed herein may comprise a processor, e.g., a computational processor, for assessing expression levels of one or more genes of the gene expression groups or NK gene subsets as disclosed herein. Such a processor may be configured with a regression model such as those disclosed herein. By inputting the marker profile (e.g., the gene expression level of certain genes in individual cells in a tumor), the processor may process the information to generate a level of interaction between the tumor and a secondary cell population and optionally determine whether a subject is an ideal candidate for a cancer therapy targeting that secondary cell population.

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure

### Introduction to the Examples

Breast cancer is the most common cancer among women. The development of breast cancer is driven by both cancer epithelial cell intrinsic factors and the tumor microenvironment. The medical treatment of breast cancer therefore targets these diverse cell populations and includes traditional chemotherapy, targeted agents inhibiting cancer cell hormone receptors, kinases, cell cycle entry, and immune cell modulators. To further improve these therapies, a deeper understanding of the cellular and molecular composition of breast tumors is required.

Single-cell RNA sequencing (scRNA-seq) technology has been applied to better characterize tumor microenvironments. In breast cancer, several scRNA-seq studies have been performed to identify key immune, cancer cell, and stromal populations of the breast tumor microenvironment. Previously studies have individually provided insight into the molecular phenotypes of cancer cells, multiple immune populations, and other stromal cells. However, they were limited by the number of samples and cells analyzed, preventing a comprehensive analysis of rare cell populations as well as the implications of cancer epithelial cell heterogeneity across a larger and more diverse set of clinical breast cancer subtypes.

For example, natural killer (NK) cells are innate lymphoid immune cells critical to anti-tumor defense. In breast cancer, they often represent 1-6% of total tumor cells. Their cytotoxic activity is regulated by a series of functionally activating and inactivating receptors. After tumor exposure, the balance of activating and inactivating receptors can change, and they can lose their cytotoxic activity, proliferative capacities, or even become tumor-promoting (10-12). Because of the small numbers of NK cells processed in most human studies, scRNA-seq analyses of NK cells often are underpowered to capture these distinct functional phenotypes.

In this computational study, an integrated atlas of 8 publicly available scRNA-seq datasets was created from samples taken from patients with early-stage breast cancer (2-9). This resource enables unbiased separation of distinct cell populations within primary breast tumors and robust characterization of phenotypes at the cell level. This integrated dataset is more statistically powerful than traditional meta-analyses of original source datasets and enables evaluation of correlations with clinical features. This resource was then used to define both cancer epithelial cell heterogeneity and rare immune cell heterogeneity along with their subsequent interactions. This comprehensive dataset of the breast tumor microenvironment provides a resource to understand the composition of breast cancer. It is the first to our knowledge to categorize NK cells in breast cancer at the single-cell level and provide evidence that cancer epithelial cell heterogeneity influences immune interactions and response to anti-PD-1 therapy.

### Example 1—an Integrated scRNA-Seq Dataset of Breast Cancer Samples Reveals Distinct Natural Killer Cell Subsets that Exhibit Diverse Functional Characteristics

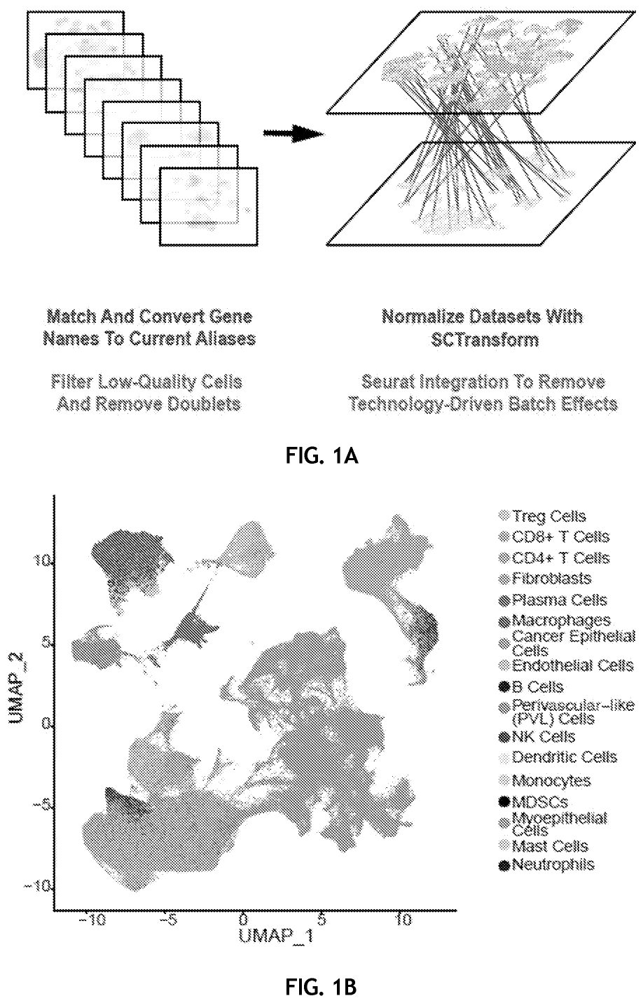

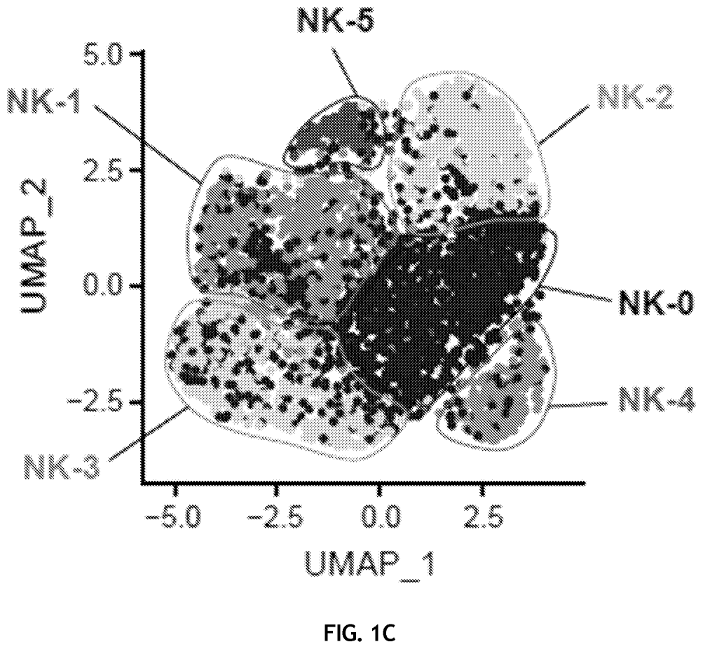

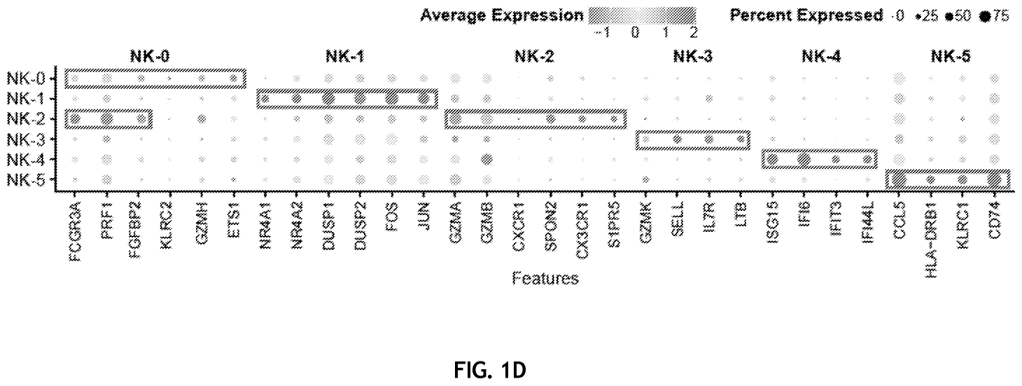

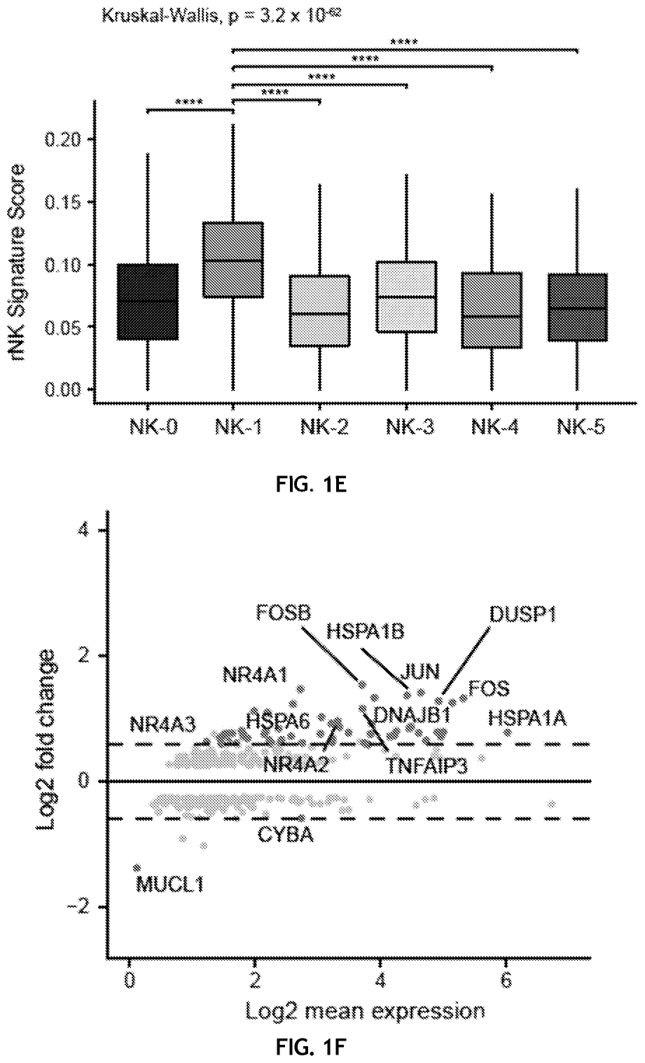

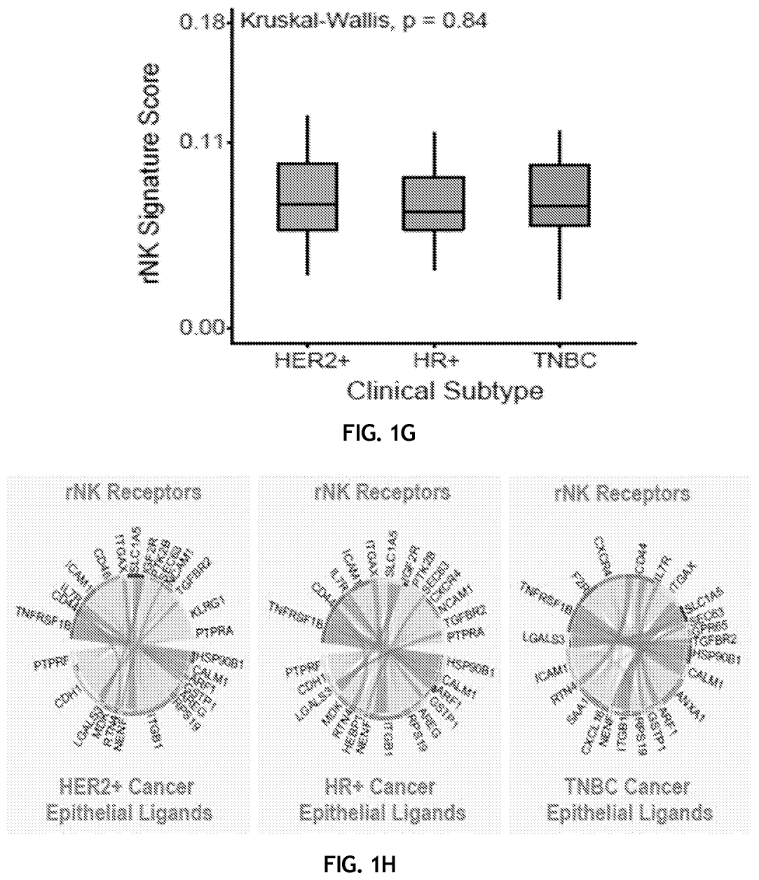

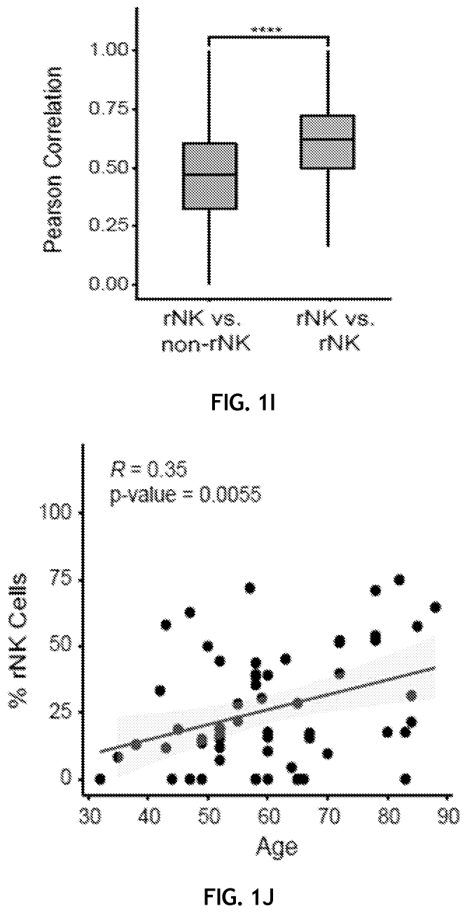

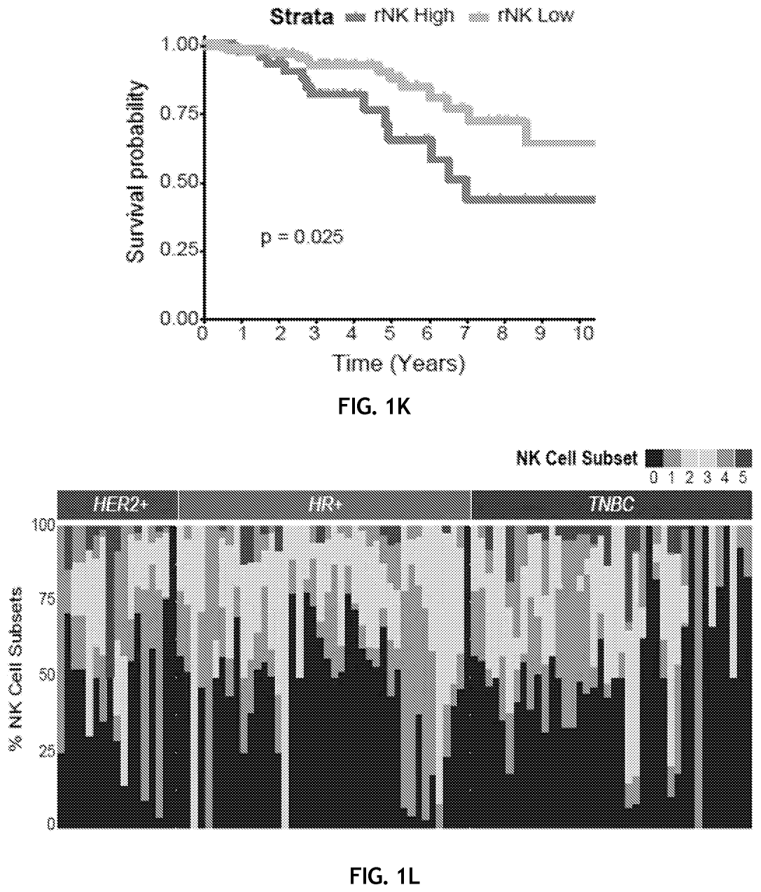

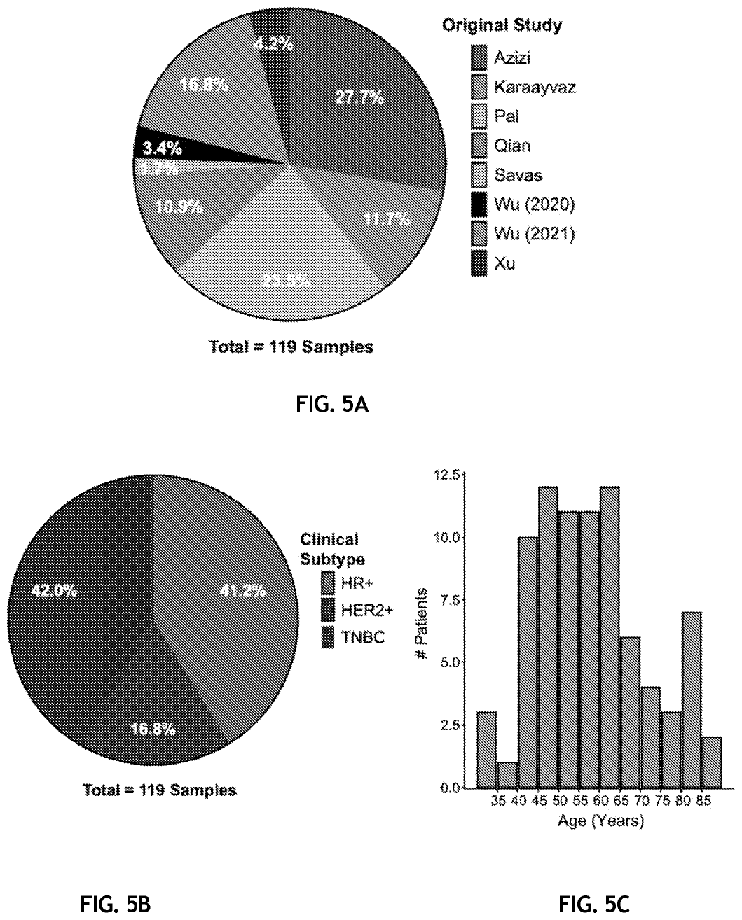

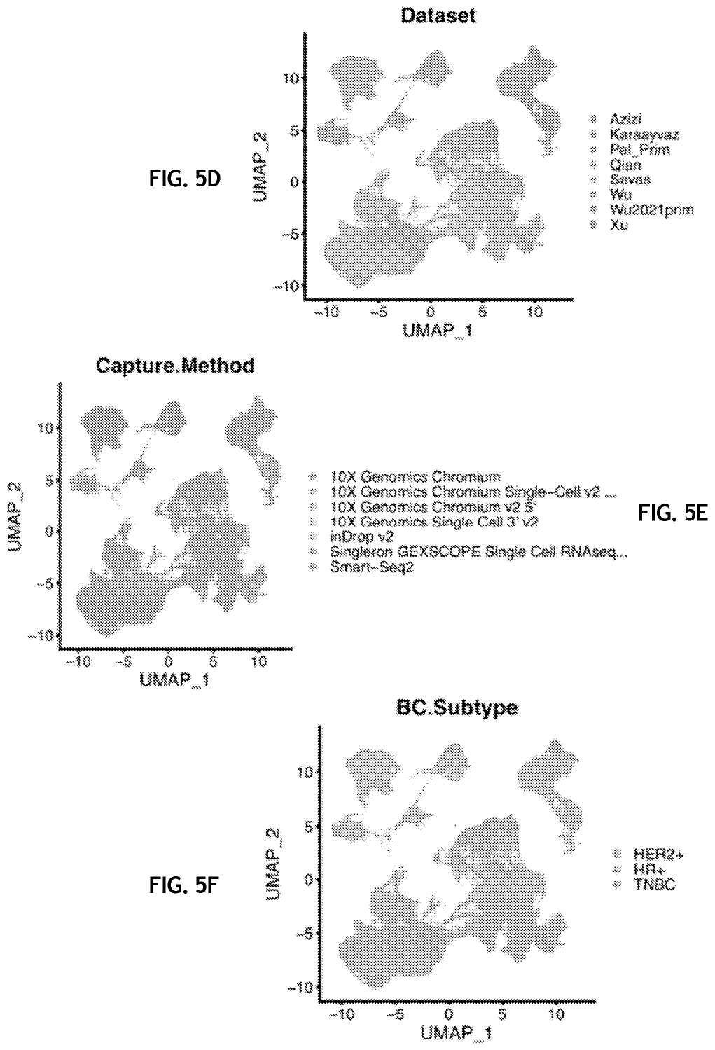

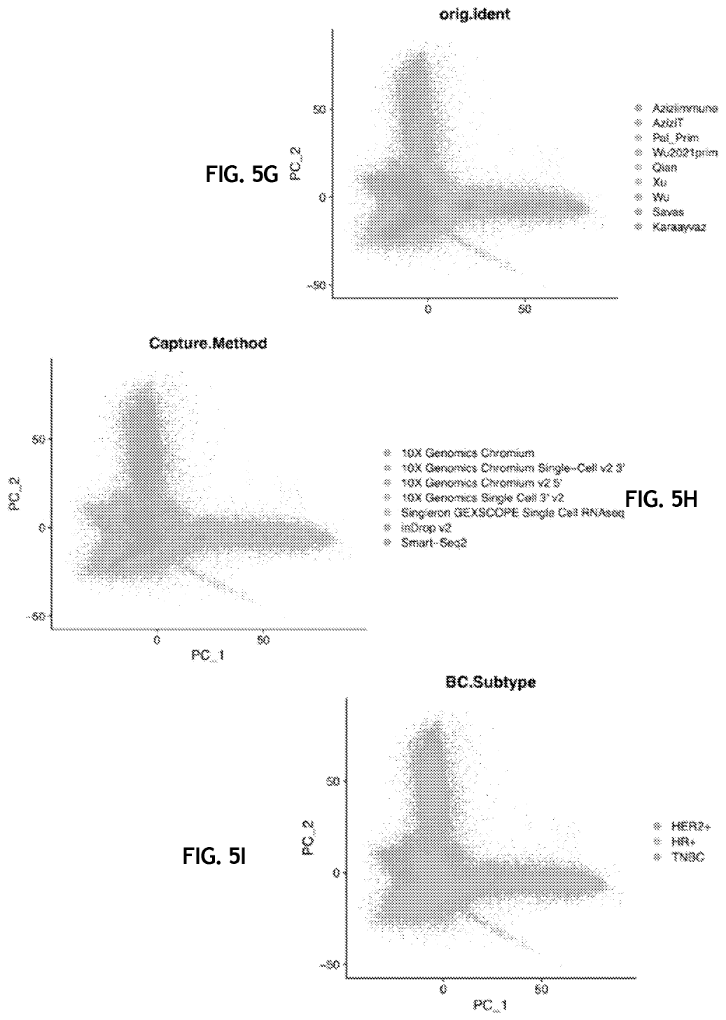

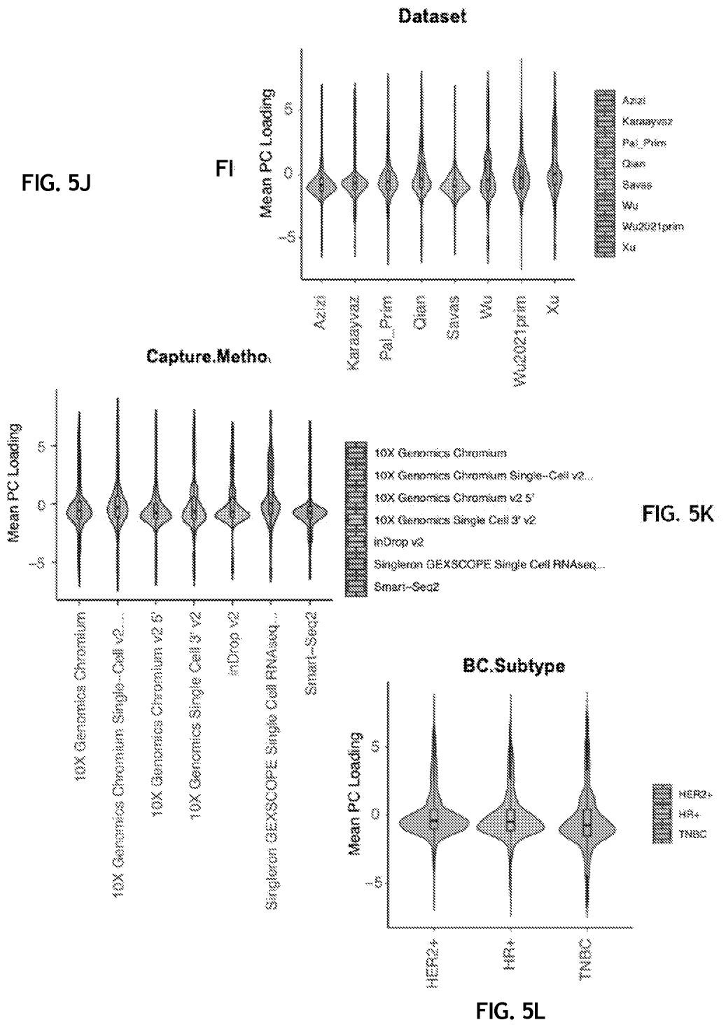

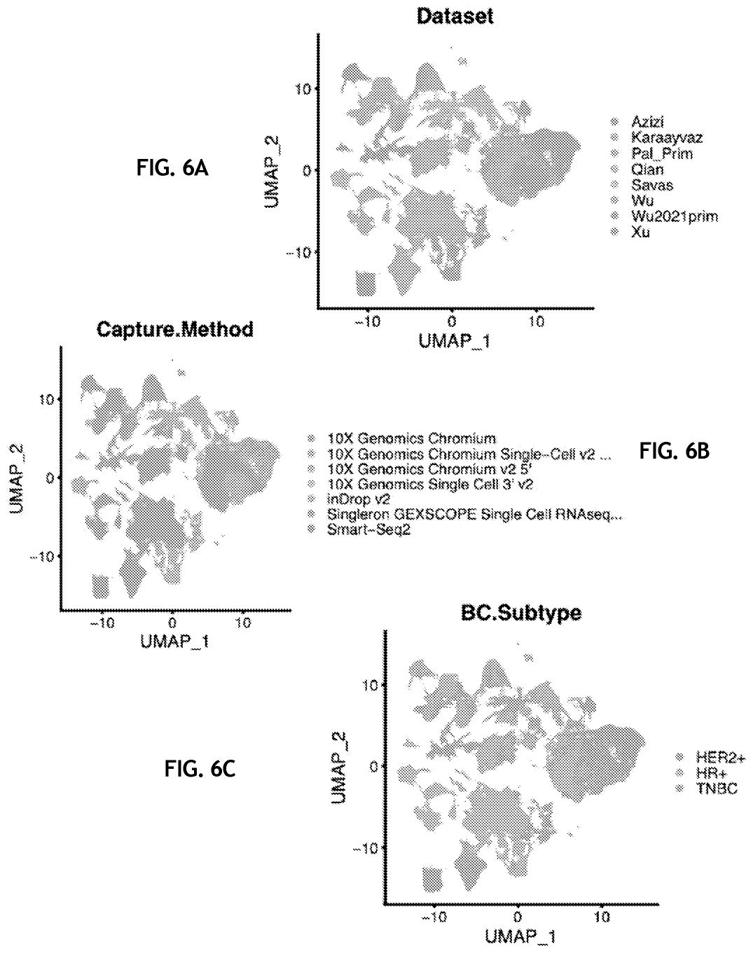

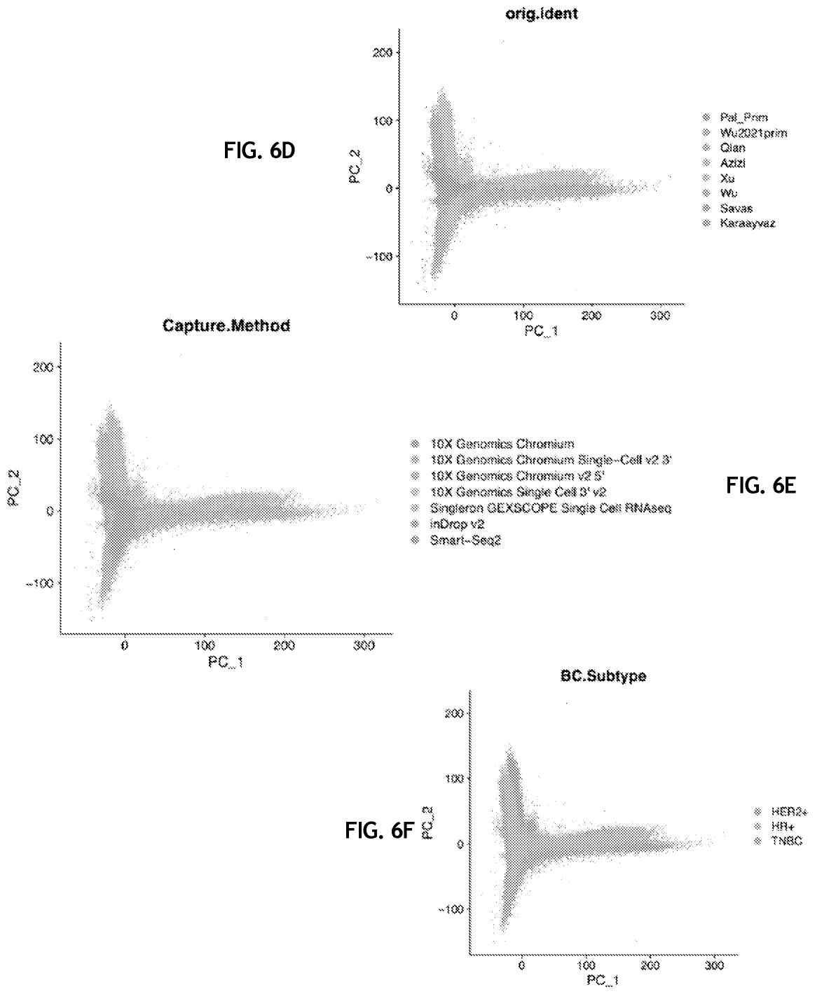

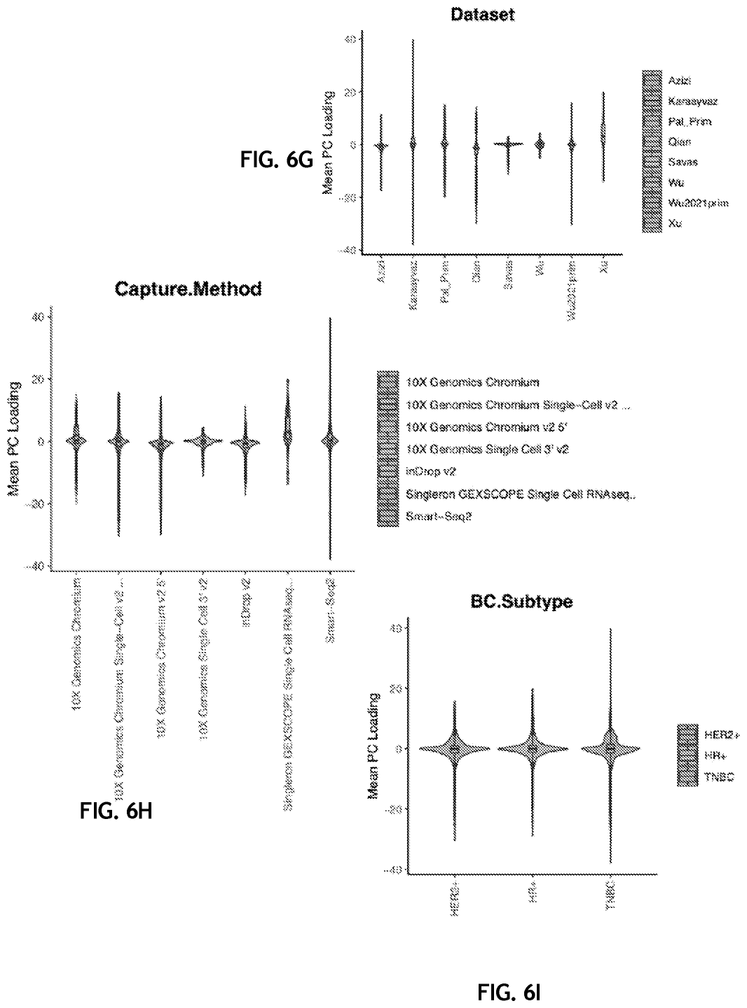

To develop a high-resolution atlas of the breast TME, we analyzed scRNA-seq data from 119 samples collected from primary tumor biopsies of 88 patients across 8 publicly available breast cancer datasets (FIG. 1A; FIG. 5A-5C) (Azizi E et al. Cell. 2018; 174 (5): 1293-308 e36; Karaayvaz M et al. Nat Commun. 2018; 9; Pal B et al. Embo Journal. 2021; 40 (11); Savas P et al. Cancer Cell. 2020; 37 (5): 623-4; Qian J B et al. Cell Res. 2020; 30 (9): 745-62; Wu S Z et al. The EMBO Journal. 2020; 39 (19); Wu S Z et al. Nature Genetics. 2021; 53 (9): 1334-47; and Xu K et al. Oncogenesis. 2021; 10 (10)). After processing each dataset separately to filter out low quality cells and doublets, we integrated a total of 236,363 cells across all clinical subtypes and a wide spectrum of clinical features. We assessed batch effect to ensure no cluster was driven by a single dataset or technology (see Methods in Example 10; FIG. 5D-5L, FIG. 6A-6I)). Cell types were identified by taking the top call resulting from a three-step process which labeled clusters based on a signature score of canonical cell markers, marker count coupled with average expression, and greatest average expression of the marker genes alone (Table 3; see Methods in Example 10).

TABLE 3
Cell Type Markers

| Cell Type | Upregulated Genes | Downregulated Genes |
|---|---|---|
| B cells | PTPRC, CD79A, CD79B, | CD33, EPCAM |
| | BLNK, CD19, MS4A1 | |
| Cancer epithelial cells | EGFR, FZR1, KRT14, ITGA6, | PTPRC |
| | KRT5, TP63, KRT17, MME, | |
| | FOXA1, GATA3, MUC1, | |
| | CD24, GABRP, EPCAM, | |
| | KRT8, KRT18, KRT19 | |
| CD4+ T cells | CD4 | None identified |
| CD8+ T cells | CD8A, CD8B | None identified |
| Dendritic cells | LILRA4, GZMB, IL3RA, | None identified |
| | CLEC9A, FLT3, IDO1, CD1C, | |
| | FCER1A, HLA-DQA1, | |
| | LAMP3, CCR7, FSCN1 | |
| Endothelial cells | PECAM1, VWF, CDH5, SELE | EPCAM, PTPRC |
| Fibroblasts | FAP, THY1, DCN, COL1A1, | None identified |
| | COL1A2, COL6A1, COL6A2, | |
| | COL6A3 | |
| Macrophages | INHBA, IL1RN, CCL4, | None identified |
| | NLRP3, EREG, IL1B, LYVE1, | |
| | PLTP, SELENOP, C1QC, | |
| | C1QA, APOE | |
| Mast cells | KIT, TPSAB1, CPA4 | None identified |
| Monocytes | FCN1, S100A9, S100A8, | HLA-DRA |
| | FCGR3A, LST1, LILRB2 | |
| Myeloid cells | PTPRC, ITGAM, HLA-DRA, | EPCAM |
| | ITGAX, CD14, FCGR3A, | |
| | CD1C, CD1A, CD68, CD33 | |
| Myeloid-derived suppressor | CD33, ITGAM, LY6G, LY6C, | CD3D, CD14, CD19, NCAM1, |
| cells | FUT4, CEACAM8, IL4R, HLA- | EPCAM |
| | DRA | |
| Myoepithelial cells | KRT5, KRT14, ACTA2, | PTPRC |
| | TAGLN, EPCAM | |
| Neutrophils | CXCR1, CD15, FCGR3A, | CD14 |
| | CSF3R, S100A9, CD24A, | |
| | TNF, CD274 | |
| NK cells | PTPRC, FGFBP2, SPON2, | CD3E, CD3G, CD33, EPCAM |
| | KLRF1, FCGR3A, KLRD1, | |
| | TRDC, NCAM1 | |
| Perivascular-like cells | MCAM, ACTA2, PDGFRB | EPCAM, PTPRC |
| Plasma cells | PTPRC, CD27, IGHA1, | CD33, EPCAM |
| | SDC1, TNFRSF17, JCHAIN, | |
| | MZB1, DERL3, CD38, | |
| | IGHG1, IGHG3, IGHG4 | |
| Regulatory T cells | FOXP3 | None identified |
| T cells | PTPRC, CD2, CD3D, CD3E, | CD33, EPCAM |
| | CD3G, CD8A, CD8B, CD4 | |

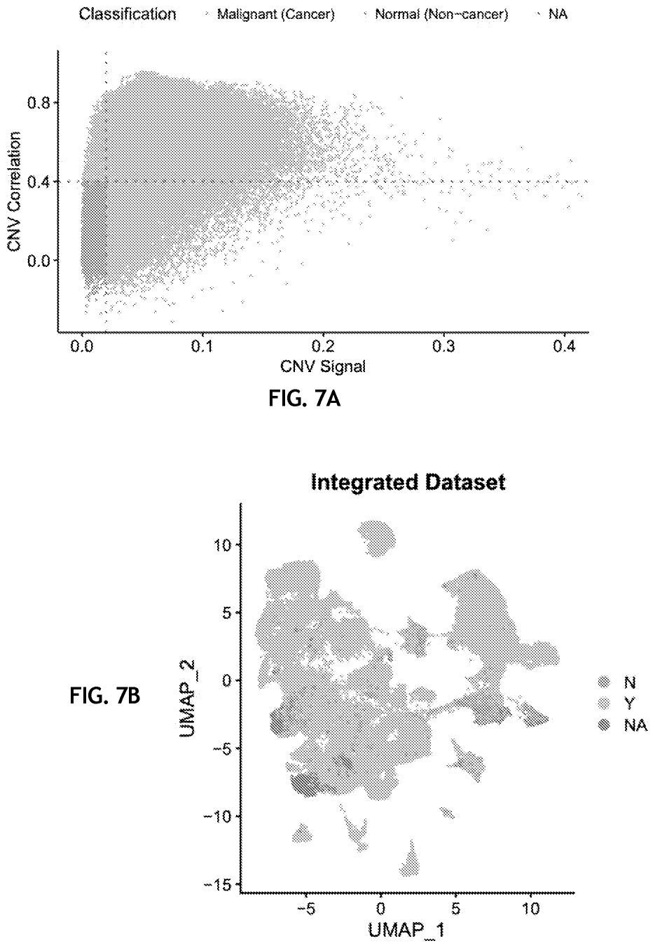

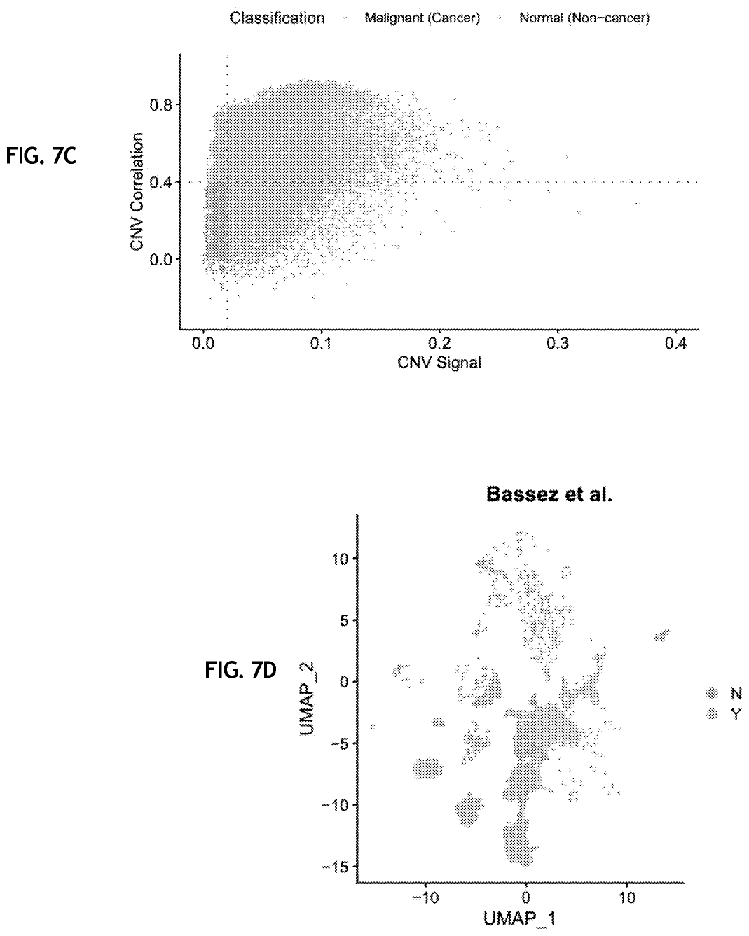

Uniform manifold approximation and projection (UMAP) visualization showed clustering of cells by lineage. Immune and stromal cell populations clustered together across clinical subtypes, while epithelial cells showed separation by subtype (FIG. 1B; FIG. 5F), which is consistent with other studies (Qian J B et al. Cell Res. 2020; 30 (9): 745-62; Wu S Z et al. Nature Genetics. 2021; 53 (9): 1334-47). For all datasets, single-cell copy number variant (CNV) profiles were estimated to distinguish cancer from normal epithelial cells (FIG. 7A-7D).

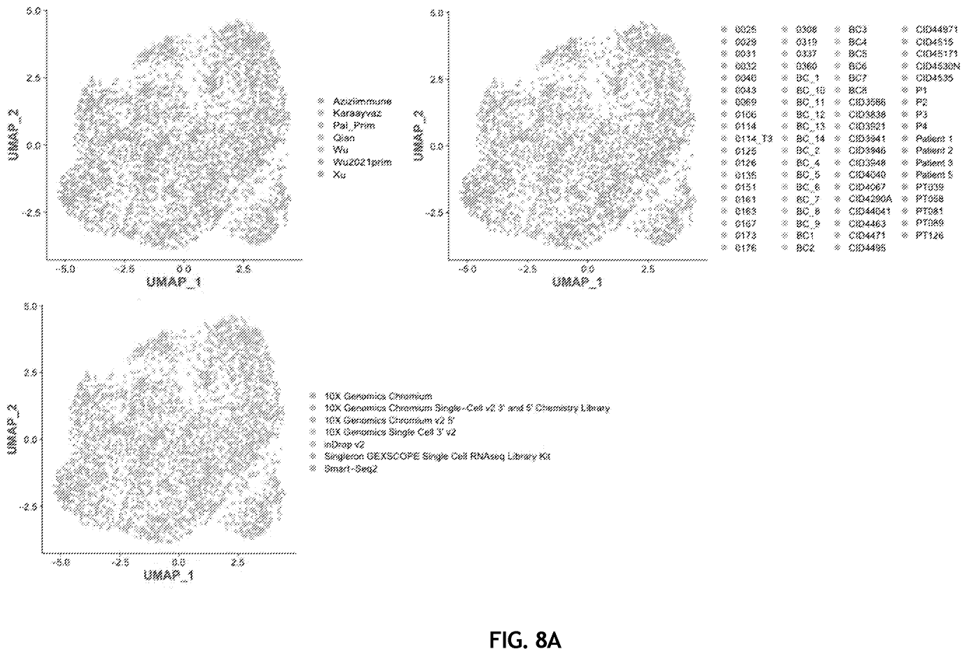

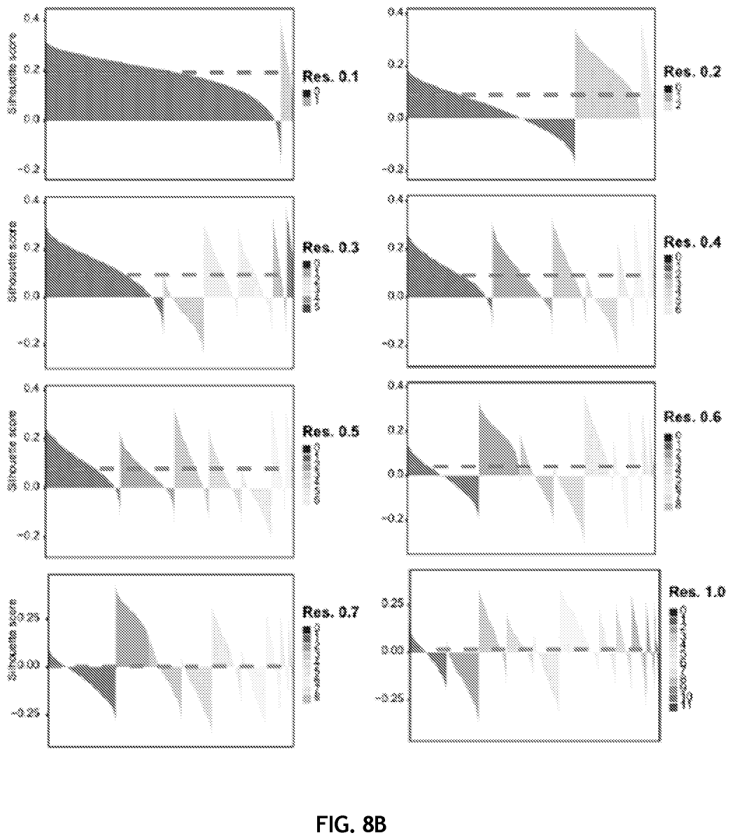

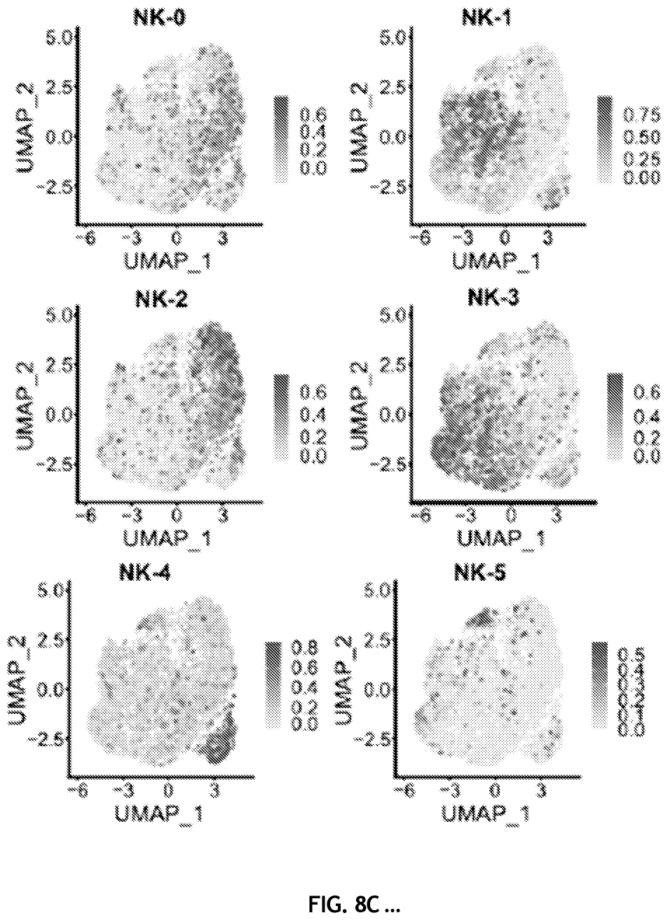

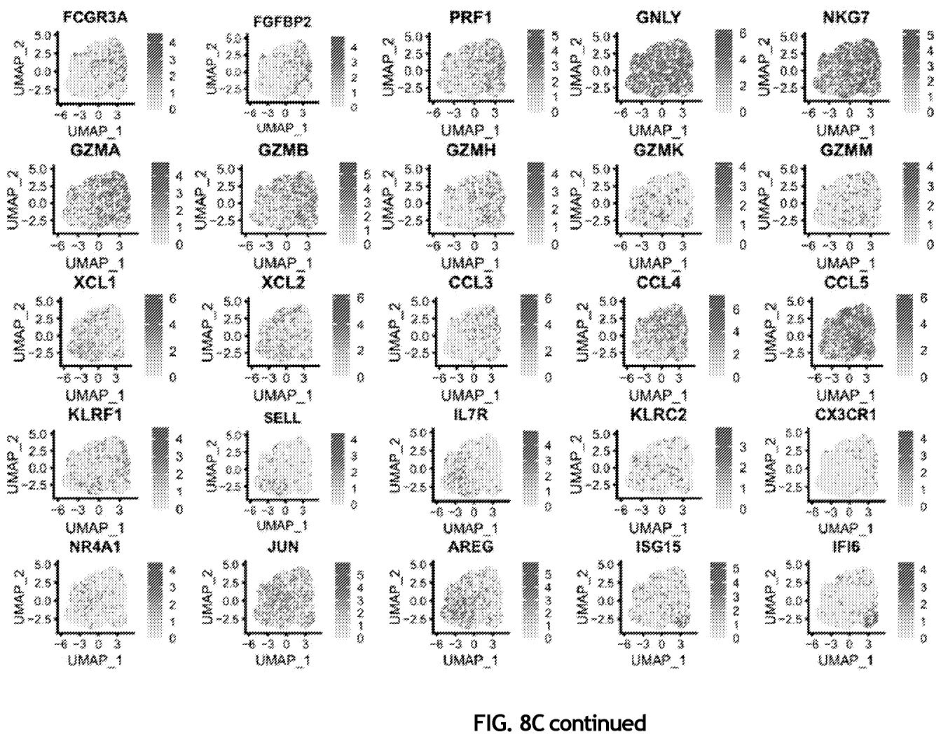

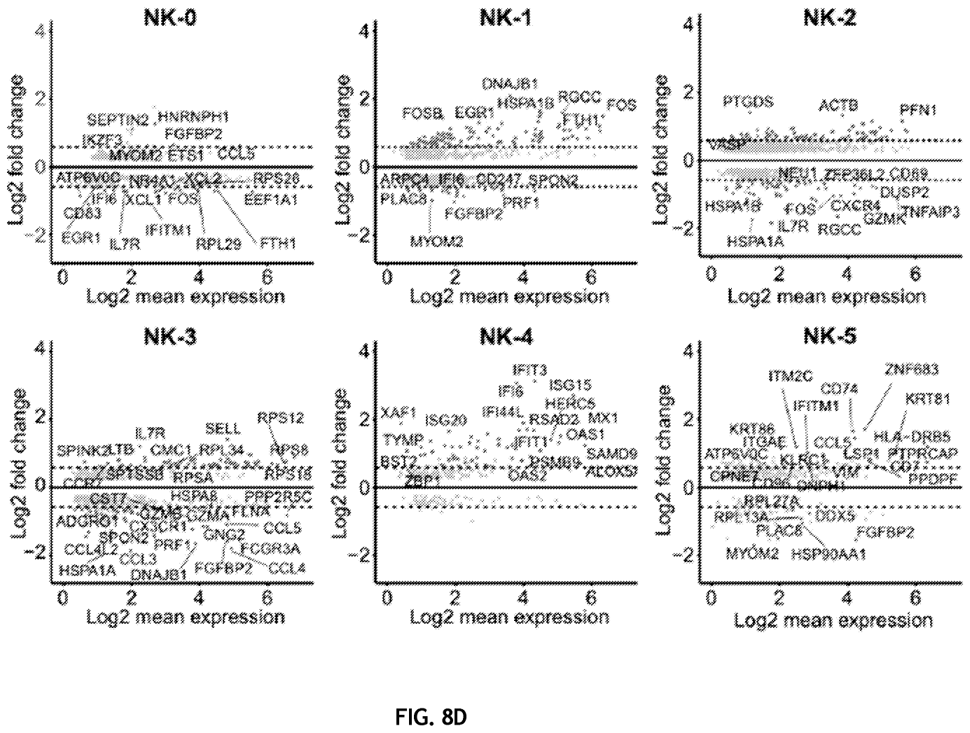

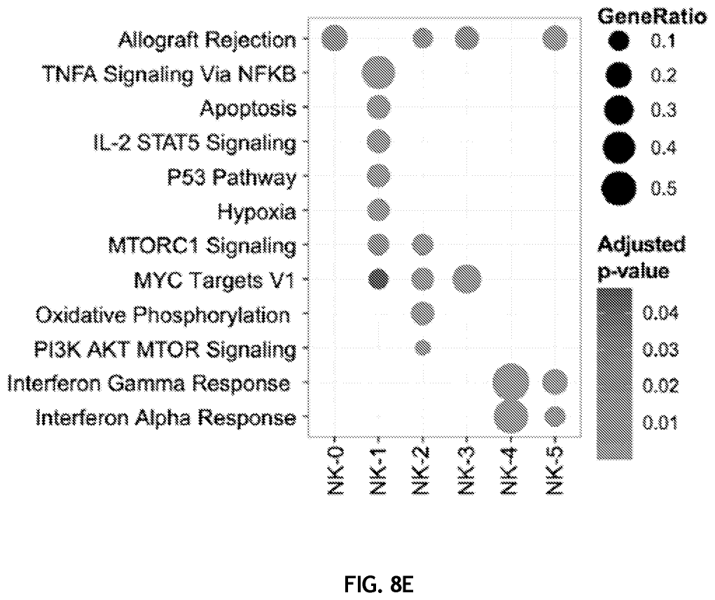

Because the number of cells in this dataset permits statistically powered analysis of rare immune cell populations in human breast cancers, we first leveraged the integrated dataset to better characterize the heterogeneity of natural killer (NK) cells. While NK cells are key mediators of anti-tumor control, our understanding of their varied phenotype and function in the breast TME is limited and incomplete. To our knowledge, there are no prior studies that dissect NK cell subsets in the human breast TME. To address this gap, we re-clustered NK cells from the integrated dataset (FIG. 8A). Unsupervised graph-based clustering uncovered 6 clusters of NK cells, designated NK-0 through NK-5 (FIG. 1C; FIG. 8B-8C).

Differential gene expression analysis between clusters revealed upregulated genes defining each NK subset (FIG. 1D; FIG. 8D; Table 4-below, see Methods in Example 10).

TABLE 4
NK Subset Markers

| NK Subset | Upregulated Genes |
|---|---|
| NK-0 | FCGR3A, PRF1, FGFBP2, GZMH, |
| | ETS1 |
| NK-1 | NR4A1, NR4A2, DUSP1, DUSP2, FOS, |
| | JUN |
| NK-2 | FCGR3A, PRF1, FGFBP2, GZMA, GZMB, |
| | CXCF1, SPON2, CX3CR1, S1PR5 |
| NK-3 | GZMK, SELL, IL7R, LTB |
| NK-4 | ISG15, IFI6, IFIT3, IFI44L |
| NK-5 | CCL5, HLA-DRB1, KLRC1, CD74, |
| | MYADM, HSPE1 |

NK-0 and NK-2 express high levels of FCGR3A (CD16) and cytolytic molecules (granzymes and PRF1), which suggests they are similar to CD56^{dim} NK. NK-0 is enriched for KLRC2, ETS1, and effector genes (GZMH, CCL5), which closely resembles gene expression profiles previously described for ‘memory-like’ NK cells. NK-2 is defined by increased expression of cytotoxicity-related genes (GZMA, GZMB, PRF1, SPON2) and S1PR5, which has been previously described in CD56^{dim} bone marrow NK cells. NK-4 is predominated by genes involved in interferon signaling (IFI6, ISG15), suggesting that this subset may be influenced by interferon-high tumor microenvironments and consists of activated NK cells involved in the direct anti-tumor response. NK-3 cells appear to have features of tissue-resident NK cells, with upregulated expression of SELL, IL7R, and GZMK, as well as reduced expression of cytolytic genes and FCGR3A (CD16). In contrast, genes of inactivity and reduced cytotoxicity were upregulated in clusters NK-1 and NK-5. NK-1 most notably was marked by genes related to the NR4A family, JUN, FOS, and DUSP1. NR4A are a family of orphan nuclear receptors which act as transcription factors; they are thought to negatively regulate T cell cytotoxicity and have been described as marking specific NK cells with reduced interferon gamma production. NK-5 had reduced expression of cytolytic genes and FCGR3A (CD16) and increased expression of KLRC1 and CD96, which are inactivators of NK cell activity. To further define the function of NK cell subsets, we performed gene set enrichment analysis of individual clusters, which confirmed their functional phenotypes (FIG. 8E).

### Example 2—Reprogrammed NK Cells are Most Similar to the NK-1 Subset and are Observed in Patient Samples Independent of Subtype

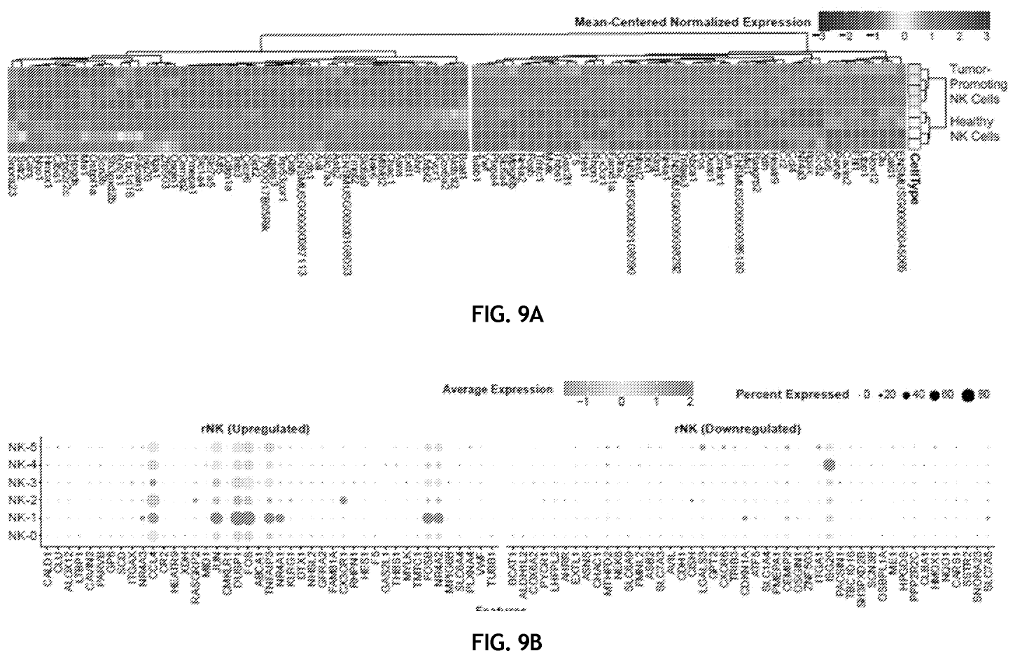

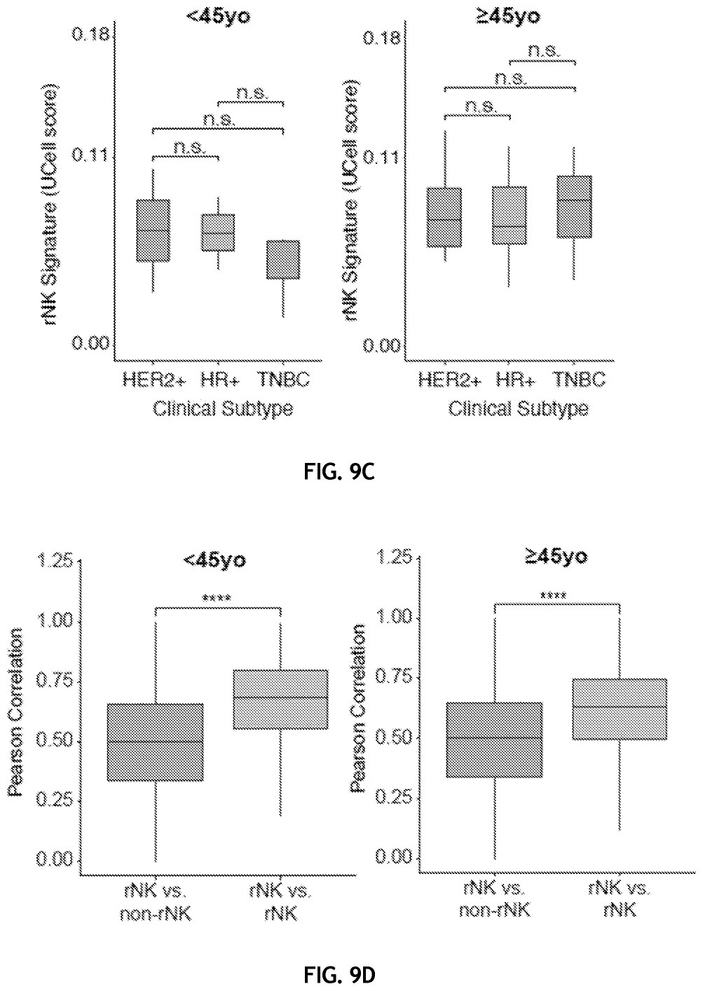

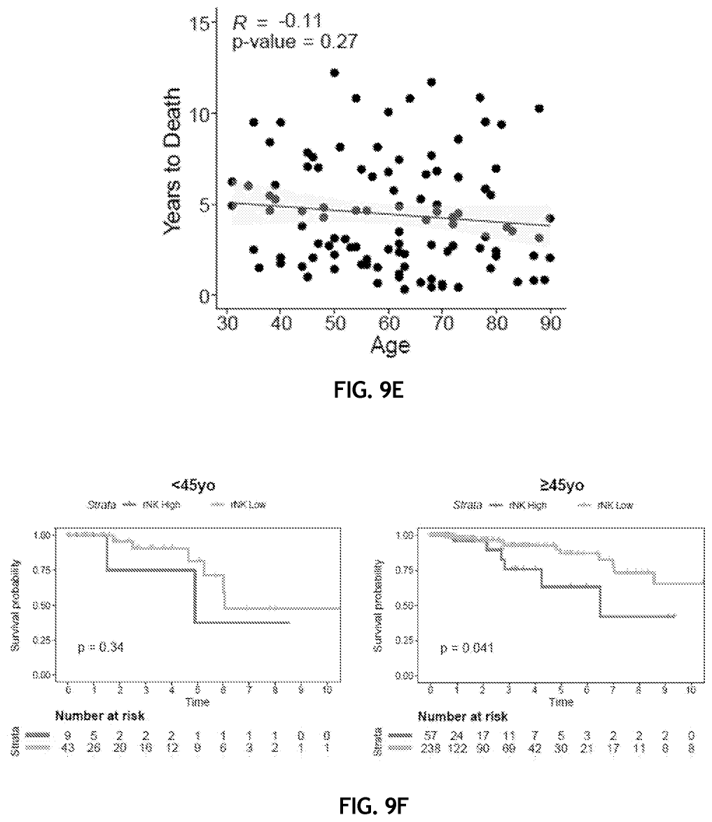

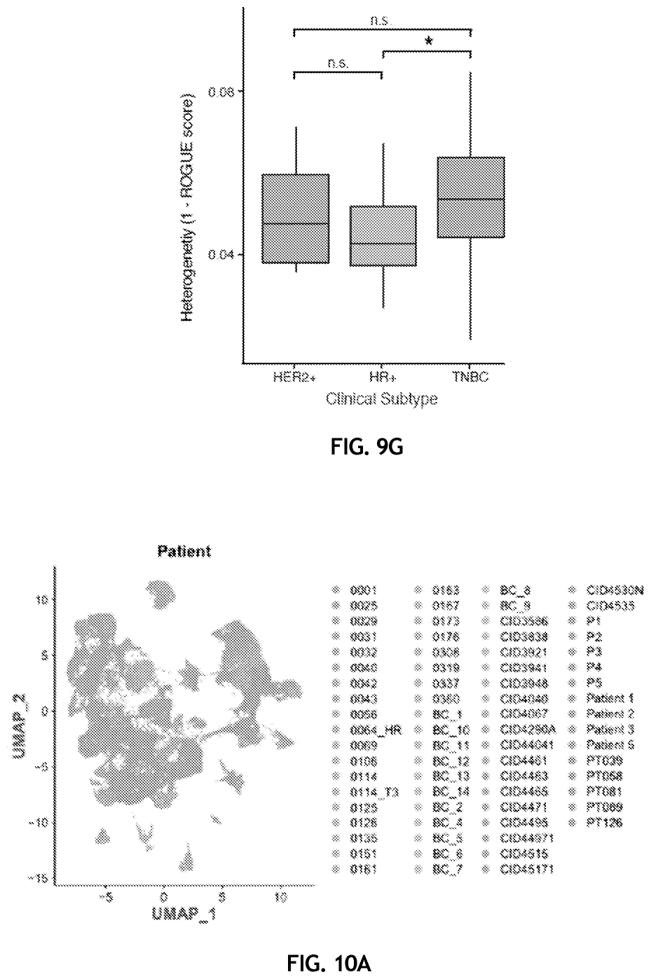

Previously in ex vivo and mouse models, we observed that NK cells can be ‘reprogrammed’ after exposure to malignant mammary epithelial cells to promote tumor outgrowth (Chan I S et al. Journal of Cell Biology. 2020; 219 (9); Chan I S et al. Journal of Clinical Investigation. 2022; 132 (6), each incorporated herein by reference in their entirety). To determine the human significance of this finding, we first generated a signature of mouse reprogrammed NK (rNK) cells based on an experiment comparing the transcriptomes of healthy NK cells to tumor-exposed NK cells that we found to be tumor promoting (FIG. 9A). We next converted the original signature to their human analogs (FIG. 9B; Table 5) and applied it to the NK cell subsets.

TABLE 5
rNK Signature

| Upregulated rNK Genes | Downregulated rNK Genes |
|---|---|
| ABCA1, ALOX12, CALD1, CAVIN2, CCL4, | AHRR, ALDH1B2, ASB2, ASNS, ATF5, AVIL, |
| CLU, CMKLR1, CR2, CX3CR1, DTX1, | BCAT1, CARS1, CDH1, CDKN1A, CEMIP2, |
| DUSP1, F5, FAM81A, FOS, FOSB, GAS2L1, | CHAC1, CISH, CLBA1, COX6A2, CXCR6, |
| GFRA2, GP6, HEATR9, HES1, ITGAX, JUN, | EXYL1, FMNL2, GPT2, HMOX1, HPGDS, |
| KLRG1, LTBP1, MID1, MPIG6B, NHSL2, | ISG20, ITGA1, LGALS3, LHFPL2, ME1, |
| NR4A1, NR4A2, NR4A3, NYLK, PARVB, | MTHFD2, NEK6, NQO1, OSBPL1A, OSGIN1, |
| PLXNA4, RASGRP2, RHPN1, SCD, SLC6A4, | PACSIN1, PMEPA1, PPP2R2C, PYCR1, |
| SLC7A5, THBS1, TMTC1, TNFAIP3, TUBB1, | RN7SL1, SCN3B, SH3PXD2B, SLC1A4, |
| VWF, XDH | SLC6A9, SLC7A3, SNORA23, SSTR2, |
| | TBC1D16, TRIB3, ZNF503 |

NK-1 scored significantly higher for the rNK signature than all other NK cell subsets (p<0.0001) (FIG. 1E). Differential gene expression analysis of rNK cells compared non-rNK cells revealed that the NR4A family (NR4A1, NR4A2, NR4A3), FOS, JUN, and DUSP1 were among the most differentially expressed genes (FIG. 1F; see Methods in Example 10), similar to the transcriptional profile of the NK-1 subset.

To test whether rNK cells were associated with a specific breast cancer subtype, we examined the expression of rNK cells across clinical subtypes. We found no significant differences in rNK cell expression across all subtypes (p>0.05; n=3,720 NK cells total) (FIG. 1G; FIG. 9C). Additionally, we found shared receptor-ligand pairs between NK cells and cancer epithelial cells across all subtypes (FIG. 1H), including LGALS3 SPN, RPS19 ICAM1, and HSP90B1_TNFRSF1B. Further, the average Pearson correlation in gene expression levels between rNK cells was greater than between rNK and non-rNK cells (p<0.0001) (FIG. 1I; FIG. 9D). Together, these findings demonstrate that rNK cells are not defined by specific breast cancer subtype biology, but suggest a shared, but still unknown mechanism, contributes to NK cell reprogramming.

To further investigate the clinical significance of rNK cells, we observed that higher expression of rNK cells correlates with older age (R=0.33, p<0.01) (FIG. 1J). Survival analysis was performed on patients in the The Cancer Genome Atlas (TCGA) breast cancer cohort, and we first confirmed that age was not a confounder of this analysis (FIG. 9E). Given the limitations of applying the rNK cell signature to bulk RNA-seq samples from TCGA which include a substantial fraction of non-NK cells, only samples with relatively high fraction of tumor-infiltrating NK cells were selected for analysis (see Methods in Example 10). Increased expression of the rNK cell signature in tumors with high fraction of NK cells correlates with worse overall survival (p<0.05) (FIG. 1K; FIG. 9F).

We then asked whether NK cell subsets were uniformly expressed across individuals and breast cancer subtypes. To answer this question, we characterized the degree of NK cell heterogeneity across patients in the integrated dataset. We observed remarkable heterogeneity in the proportions of NK cell subsets across patients (FIG. 1L). Additionally, all NK cell subsets were found within each individual tumor sample. However, NK cell subset heterogeneity as quantified using ROGUE analysis was observed to be significantly higher in certain clinical subtypes than others (FIG. 9G). While there have been multiple reports of NK cell subsets in other cancers, no other studies have explored the diversity of NK cell subsets within individual patient samples. Our findings provide further evidence of the diverse phenotypes of NK cells within individual primary breast tumors.

### Example 3—Individual Breast Tumors have Varying Degrees of Cancer Epithelial Cell Heterogeneity

Because we observed that NK cell heterogeneity is associated with certain clinical subtypes of breast cancer (FIG. 1L), we reasoned that heterogeneity within breast cancer subtypes would be important in further characterizing the breast tumor microenvironment (TME). We then used our dataset to explore the heterogeneity of cancer epithelial cells at different resolutions: At the level of single gene expression, molecular subtypes, and then 10 categories of cancer epithelial cells that reflect intratumoral transcriptional heterogeneity (ITTH).

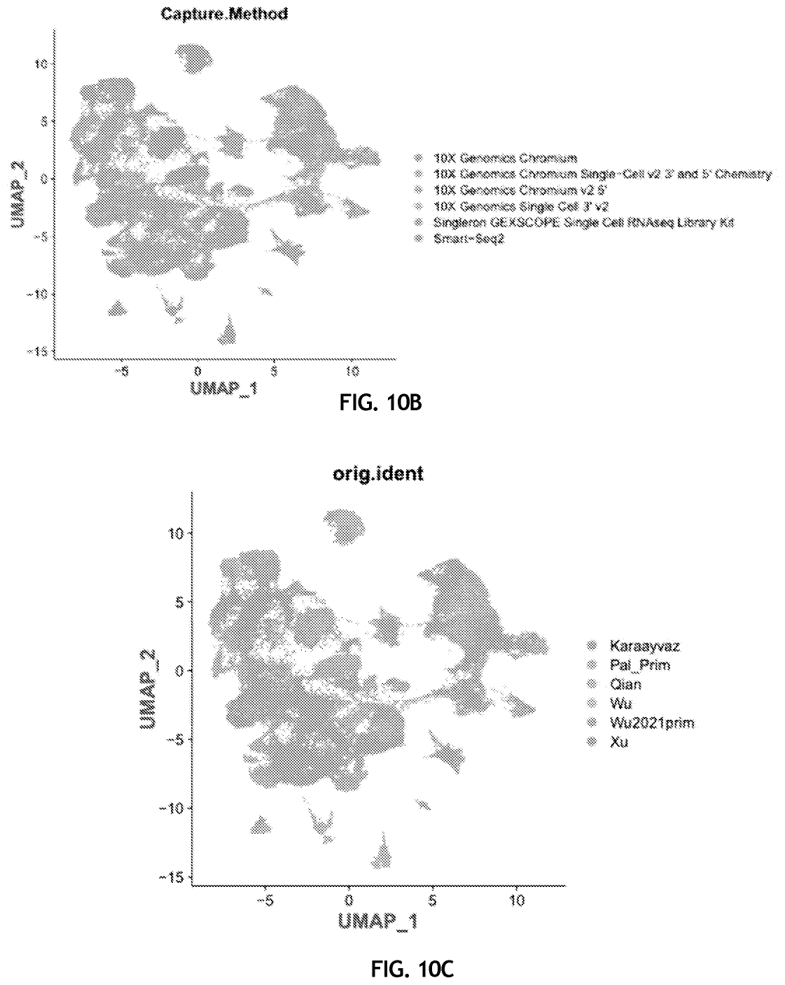

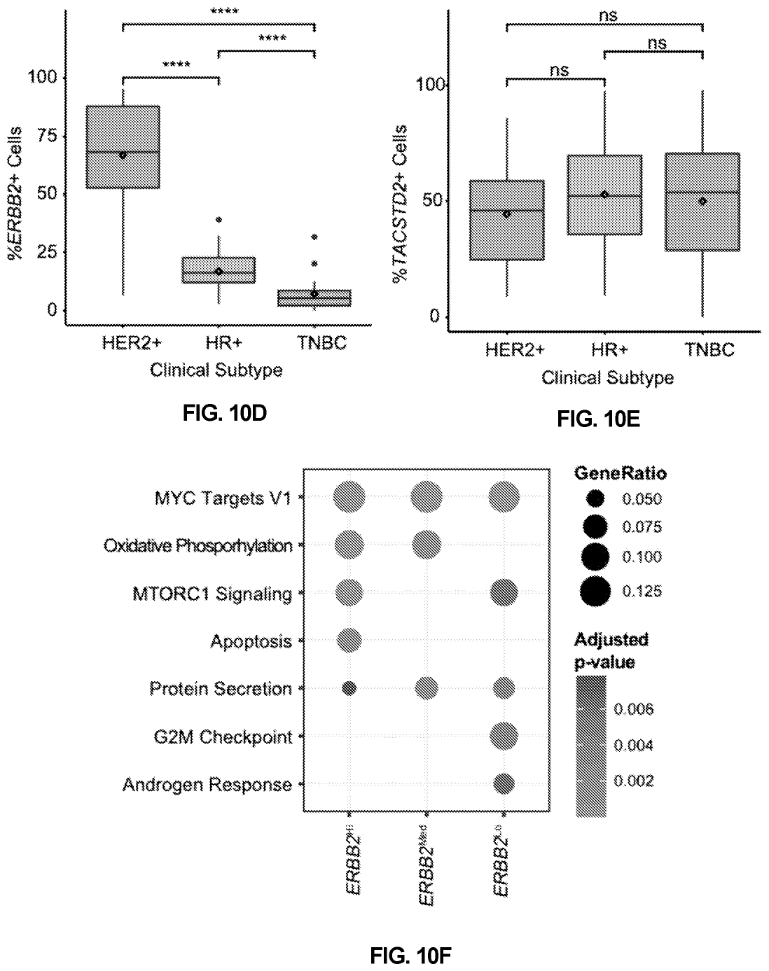

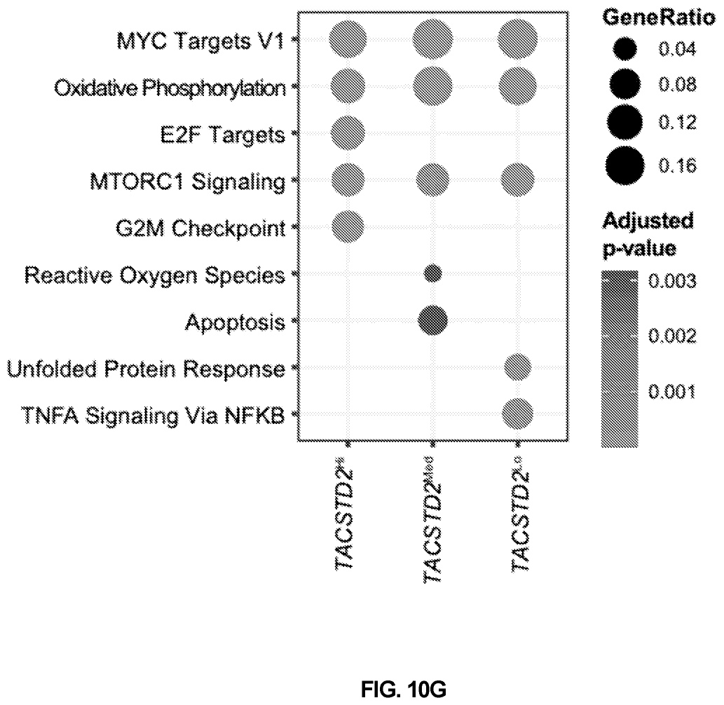

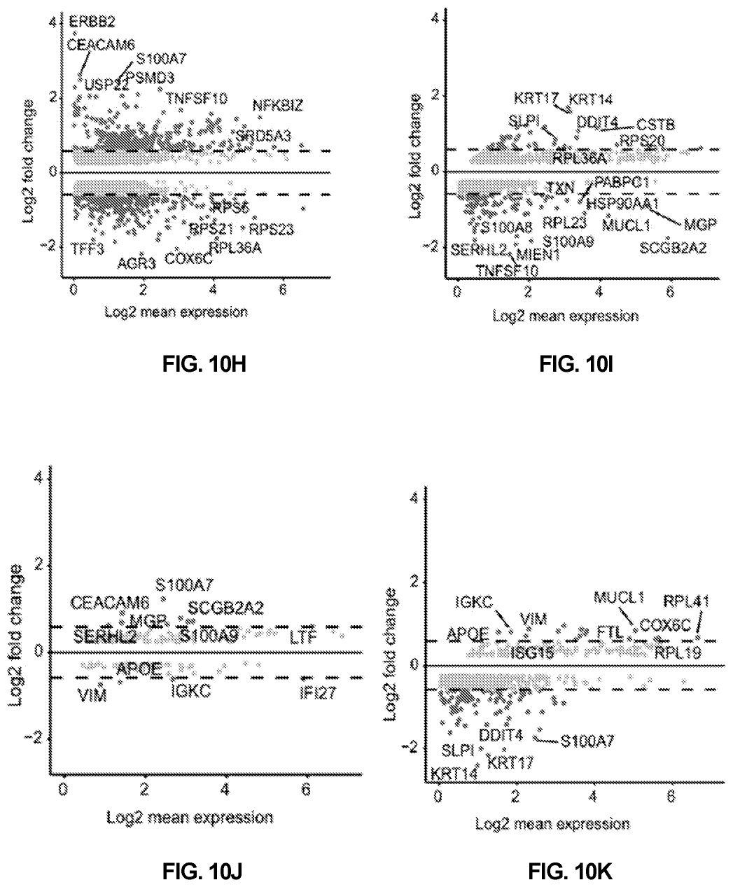

Cancer epithelial cells are well known to demonstrate substantial intertumoral and intratumoral heterogeneity in primary breast tumors at the single gene level. For example, heterogeneous expression of ERBB2 (HER2) and TACSTD2 (TROP2) could have clinical implications. Newer anti-HER2 and anti-TROP2 agents have shown benefit in patients across clinical breast cancer subtypes. This highlights an urgent need to better understand HER2 and TROP2 expression heterogeneity in cancer epithelial cells to improve patient selection. In contrast to bulk RNA-seq, which aggregates expression levels across all cell types and thus offers limited resolution for studying intratumoral heterogeneity, the integrated dataset can be used evaluate HER2/ERBB2 and TROP2/TACSTD2 heterogeneity in cancer epithelial cells at the single-cell level across tumor samples. To do so, epithelial cells in the integrated dataset were re-clustered and re-integrated to account for technology-driven batch effects (FIG. 10A-10C). Cancer epithelial cells were distinguished from normal epithelial cells (FIG. 7A-7B). Consistent with prior studies, epithelial cells demonstrated stratification by patient (FIG. 10A).

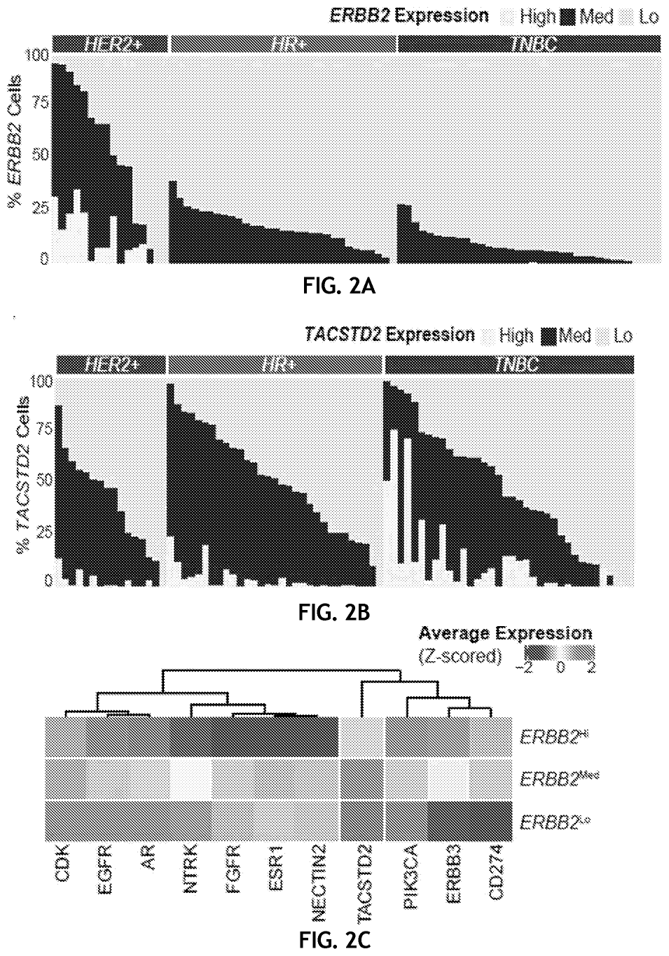

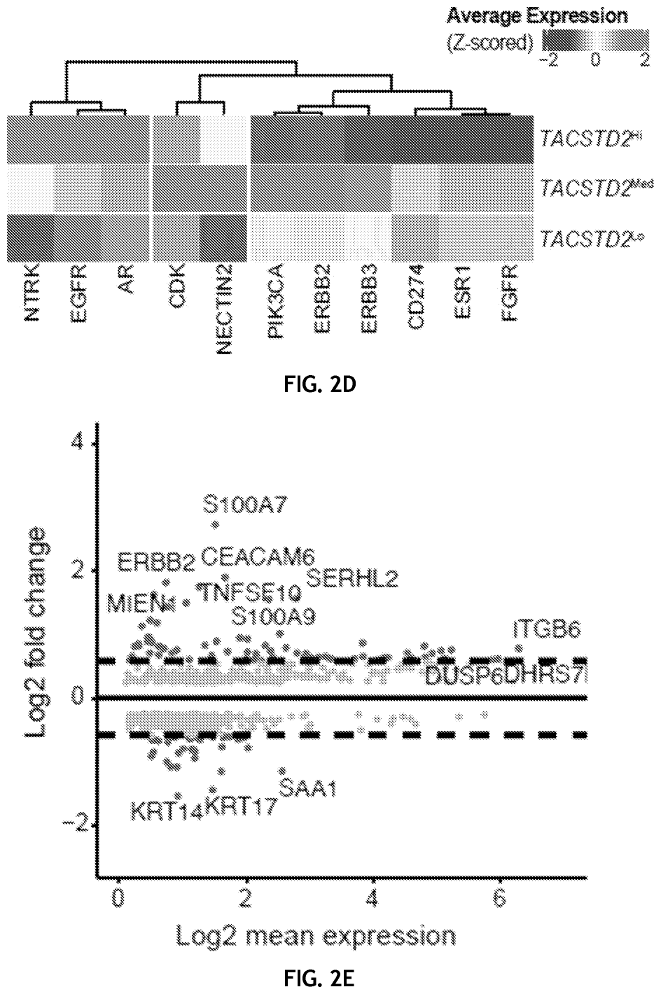

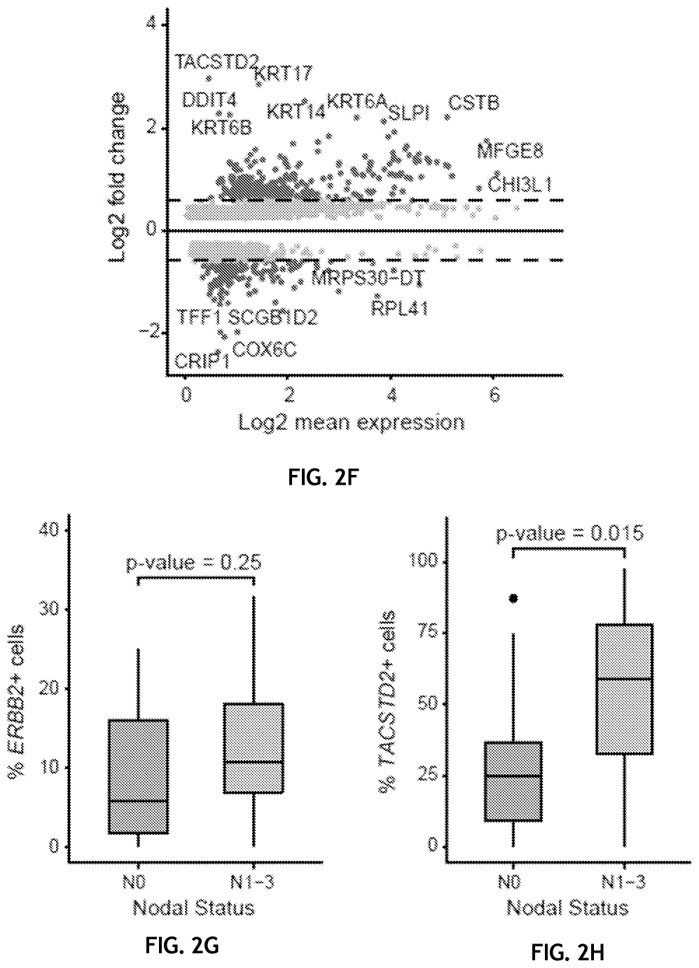

Previous bulk RNA-seq and immunohistochemistry (IHC) studies have reported expression of the ERBB2 gene or HER2 protein in up to 70% of HER2-negative breast tumors (Tan RSYC et al. BMC Medicine. 2022; 20 (1); Schettini F et al. npj Breast Cancer. 2021; 7 (1)). We detect ERBB2 expression in 92% of samples independent of clinical subtype at the single-cell level (FIG. 2A, FIG. 10D). For TACSTD2, we similarly observed notable heterogeneity (FIG. 2B, FIG. 10E). In particular, TACSTD2 expression was observed across all subtypes in 94% of samples. This provides additional evidence at single-cell resolution to what has been previously described in bulk RNA-seq and IHC studies, which report TROP2 positivity in 50-93% of breast cancer samples. Interestingly, the proportion of ERBB2^{Hi} and ERBB2^{Med} cells and TACSTD2^{Hi} and TACSTD2^{Med} cells varied between samples, suggesting additional factors could help determine patient selection for anti-HER2 and anti-TROP2 antibody drug conjugates. We next asked how other clinically relevant target genes were related to ERBB2 expression. We found that PIK3CA, ERBB3, and FGFR expression were highest in ERBB2^{Hi} cells (FIG. 2C). In contrast, TACSTD2 and CD274 expression was highest in ERBB2^{Med} cells and notably lower in ERBB2^{Hi} cells. Upon analysis of target genes related to TACSTD2, we found EGFR, CDK, and NTRK expression were elevated in TACSTD2^{Hi} cells (FIG. 2D). ERBB2, ERBB3, PIK3CA, and AR expression were highest in TACSTD2^{Med} cells. Additionally, we observed that TACSTD2^{Med} cells highly express NECTIN2, a ligand related to TIGIT, which hints at potential synergy with anti-TROP2 therapeutics and immune checkpoint inhibition.

Next, we characterized the heterogeneity of molecular features between ERBB^{Hi}, ERBB2^{Med}, and ERBB2^{Lo} populations. We performed gene set enrichment analysis for the ERBB2 and TACSTD2 groups to further characterize function (FIGS. 10F-10G) and differential gene expression analyses between the groups (FIG. 2E; FIGS. 10F-10G). Of the upregulated genes for ERBB2^{Hi} cells, 47 genes have been shown to be direct interactors with ERBB2 (Table 6, below).

TABLE 6
Upregulated Genes in ERBB2^{HI} cells.

| | |
|---|---|
| Upregulated Genes in | ERBB2, PSMD3, MUCL1, GRB7, HSP90AA1, |
| ERBB2^{Hi} cells that | MAP2K3, CDH1, HSP90B1, PIK3R1, |
| are direct interactors | PABPC1, C11orf58, CDK12, ZMYND8, TXN, |
| with ERRBB2 | TLN1, SDC1, C4orf3, HSPA5, AP2B1, |
| | C1orf43, TOP1, INSR, CLTC, CALM1, |
| | HSP90AB1, LGALS8, EZR, C1orf21, PAK1, |
| | KPNB1, HDLBP, PTPN11, C4ORF3, PGAM1, |
| | MCL1, STMN1, C1ORF43, ARF5, C1ORF21, |
| | C11ORF58, SOCS3, GAPDH, IL6ST, ELF3, |
| | ESR1, ALDOA, TIMP1 |

Differentially expressed genes in ERBB2^{Med} cells compared to ERBB2^{Hi} and ERBB2^{Lo} cells may provide insight into molecular features associated with ERBB2 heterogeneity and HER2-low tumors (FIG. 2E, FIG. 10H-10I). For instance, CEACAM6 (Rizeq B et al. Cancer ScL 2018; 109 (1): 33-42), DUSP6, and ITGB6 were found to be upregulated in in ERBB2^{Med} cells and may add to the growing literature on the underlying biology of HER2-low tumors (FIG. 2E).

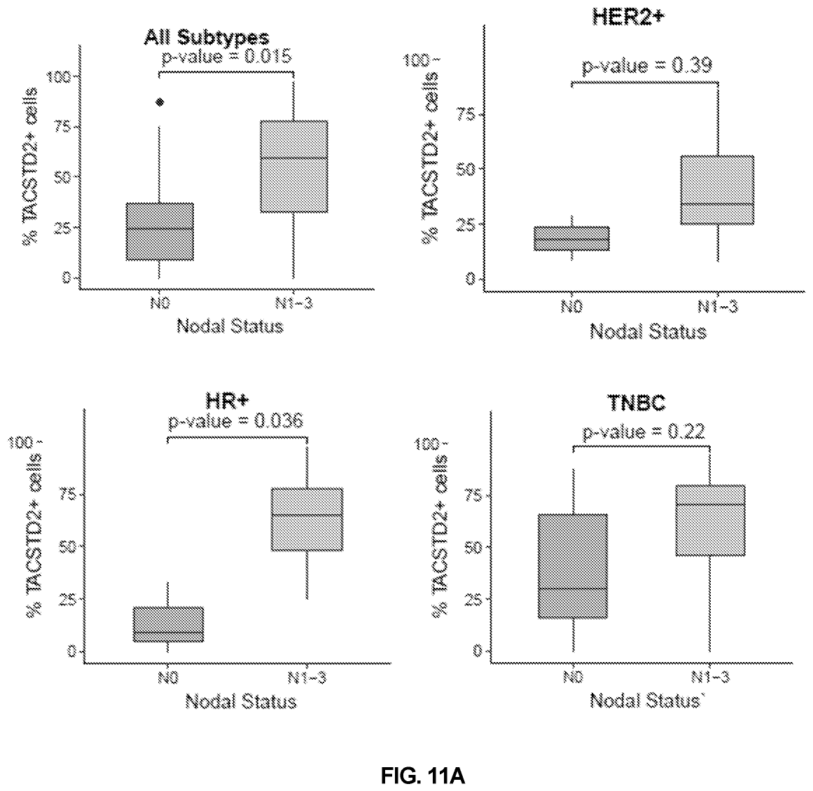

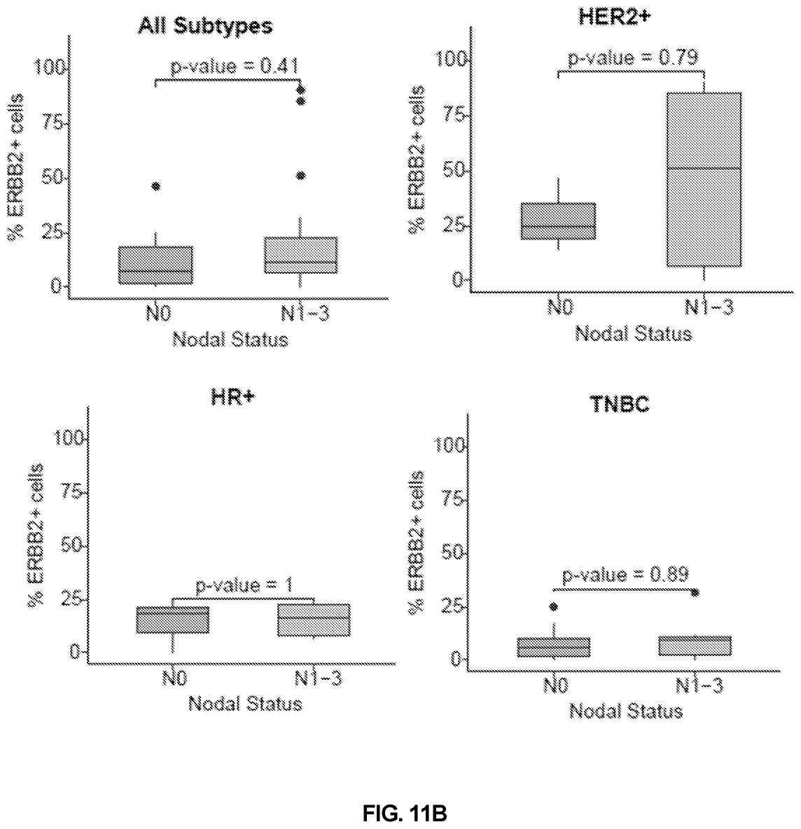

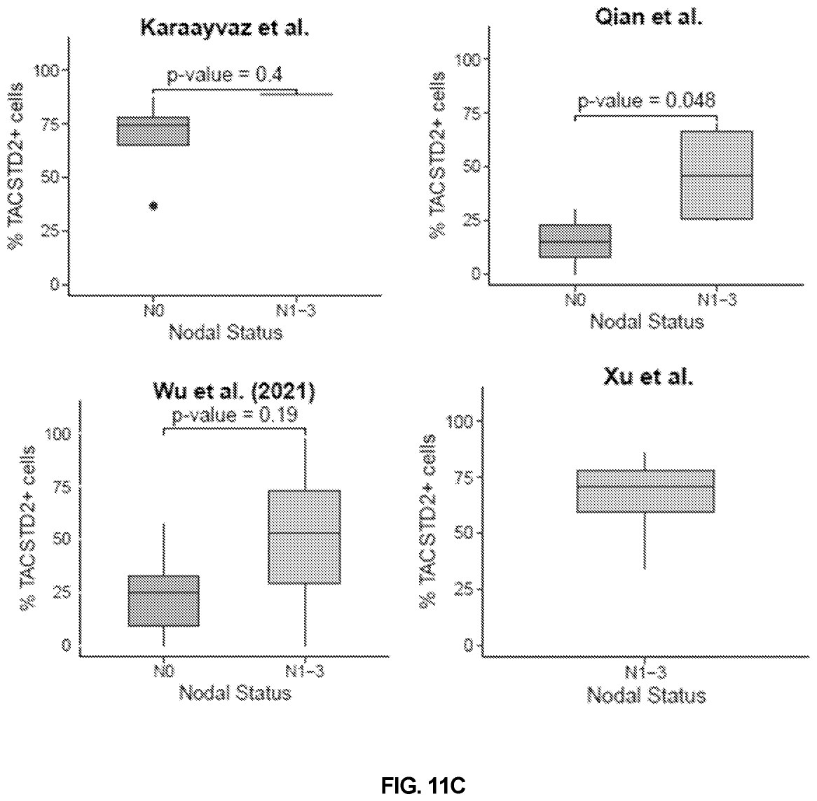

For TACSTD2^{Hi}, TACSTD2^{Med}, and TACSTD2^{Lo} cells, differential gene expression analyses (FIG. 2F; FIG. 10J-10K) identified KRT14 and KRT17 as significantly upregulated genes in TACSTD2^{Hi} cells. These genes have been implicated as markers for highly metastatic breast cancer cells (Cheung K J et al. Cell. 2013; 155 (7): 1639-51). Interestingly, when assessing for correlation with clinical features, ERBB2-enriched non-HER2+ tumors did not show significant association with higher nodal status (p=0.25) (FIG. 2G; FIG. 11A-11B). However, TACSTD2-enriched tumors were significantly associated with higher nodal status (p=0.015) (FIG. 2H). When performing this analysis separately in each cohort, the combined result by Fisher's combined probability was not statistically significant, though it trended toward significance (X=11.227, p=0.08) (FIG. 11C). This again highlights the value of our data integration approach, which creates a more statistically powered dataset and enables evaluation of correlations with clinical features over traditional meta-analysis methods.

Our study joins several reports noting the heterogeneous expression of single genes within single tumors (Seol H et al. Modern Pathology. 2012; 25 (7): 938-48; Janiszewska M et al. Nature Genetics. 2015; 47 (10): 1212-9; Soucheray M et al. Cancer Research. 2015; 75 (20): 4372-83; Muzumdar M D et al. Nature Communications. 2016; 7 (1): 12685). Recognizing that intratumoral heterogeneity occurs beyond single genes, we next characterized the ITTH of cancer epithelial cells in primary breast tumors. To do so, we applied a well-characterized subtype classifier (Wu S Z et al. Nature Genetics. 2021; 53 (9): 1334-47) which scores the four molecular subtypes (Luminal A, Luminal B, Her2, Basal) to cancer epithelial cells in the integrated dataset. We found that each patient tumor expressed differing proportions of cells from each molecular subtype with varied degree of concordance with the clinical subtype diagnosis (FIG. 2I). This finding prompted us to explore how cancer epithelial cell ITTH may be influenced by features beyond molecular subtype. We quantified the degree of heterogeneity across all cancer epithelial cells in a patient tumor using ROGUE analysis (FIG. 2I) (Liu B et al. Nat Commun. 2020; 11 (1): 3155). The ROGUE score for each individual tumor sample also reflected molecular subtype heterogeneity to some degree; however, we noticed discordance in 33.3% of samples, which demonstrated homogeneity based on molecular subtype but high heterogeneity based on ROGUE score (FIG. 2J; see Methods in Example 10). This suggests that other factors beyond molecular subtype-associated genes drive the observed heterogeneity and underscores a need for different approaches to study cancer epithelial cell ITTH at higher resolution than that of existing subtype classifiers.

### Example 4—Cancer Epithelial Cell Heterogeneity can be Defined by 10 Unifying Groups of Gene Signatures

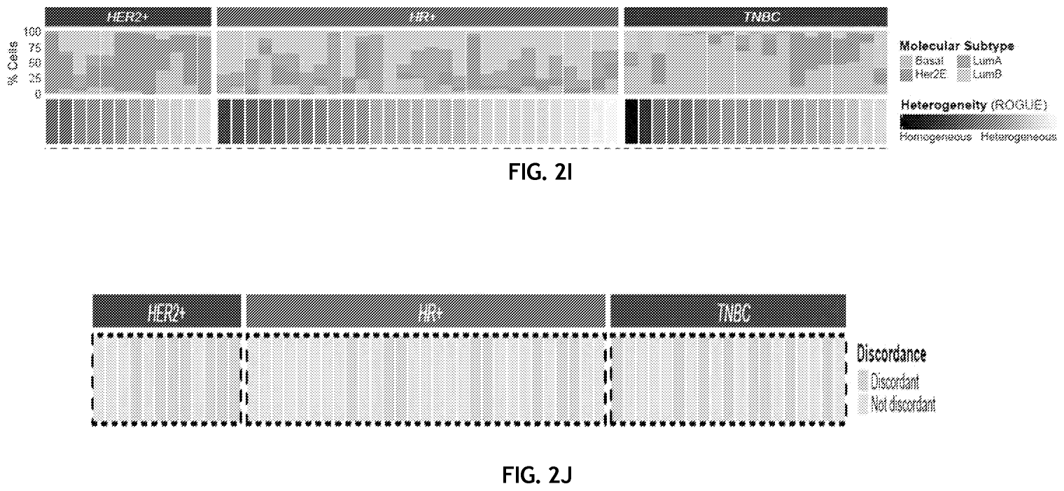

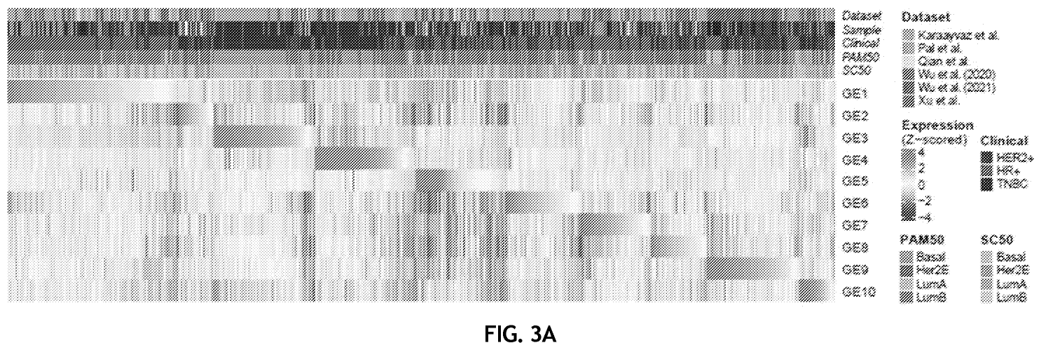

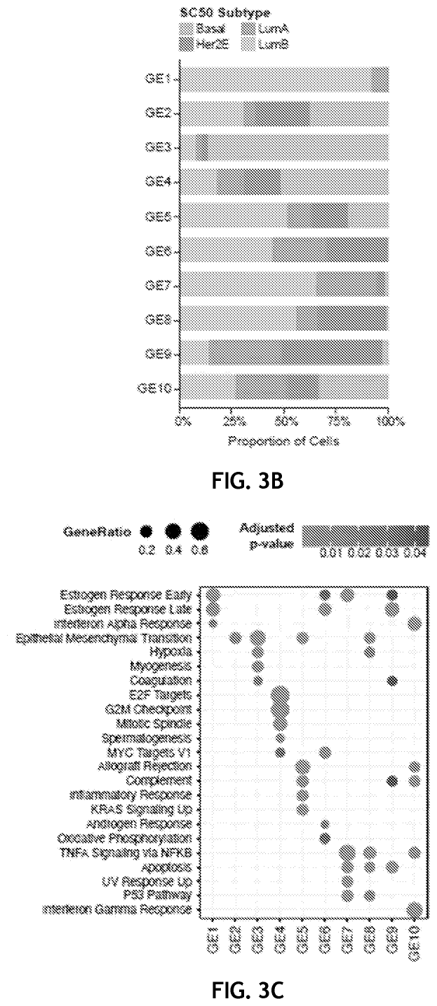

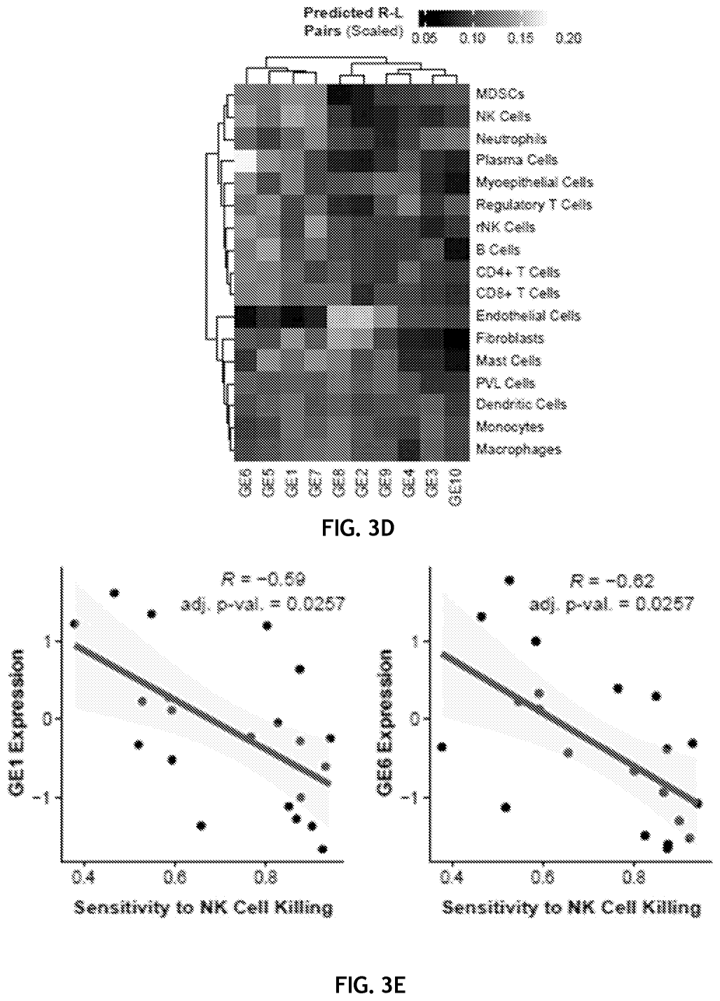

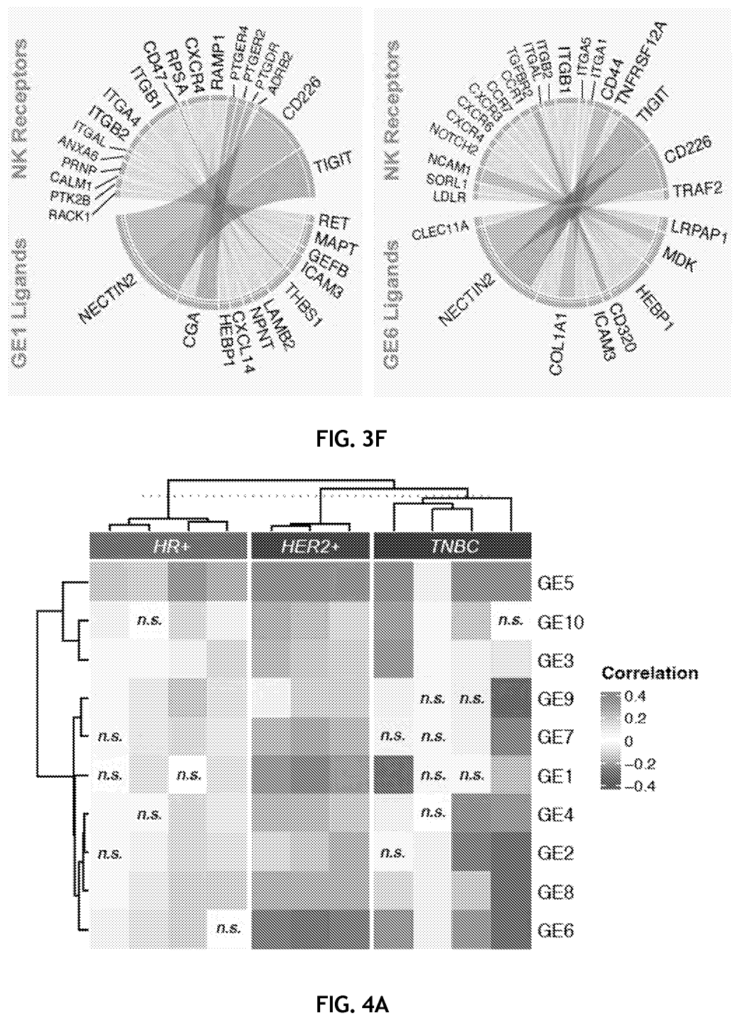

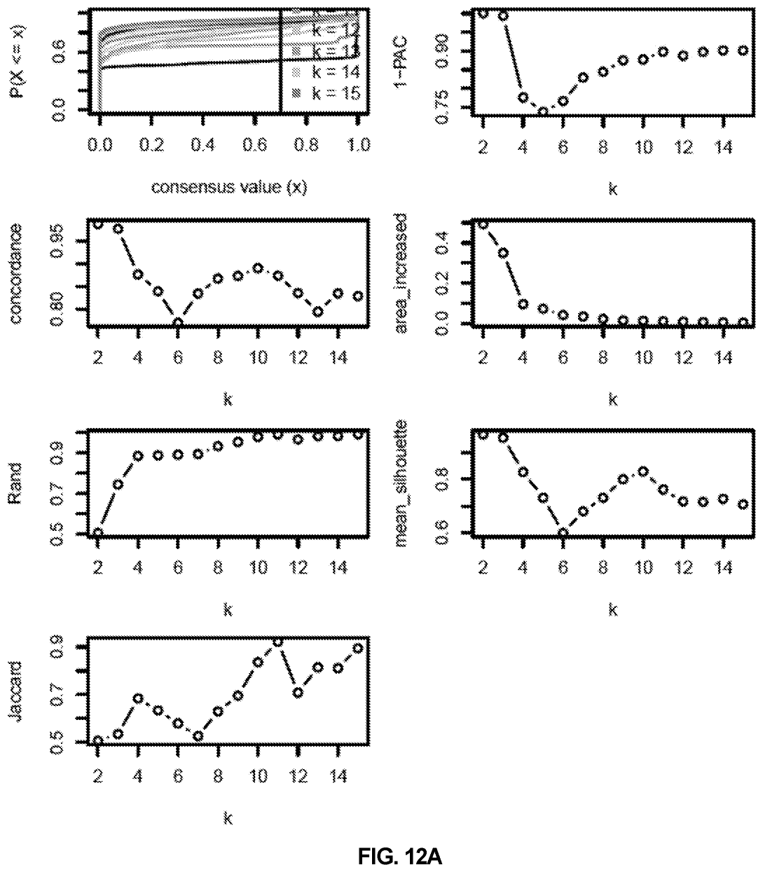

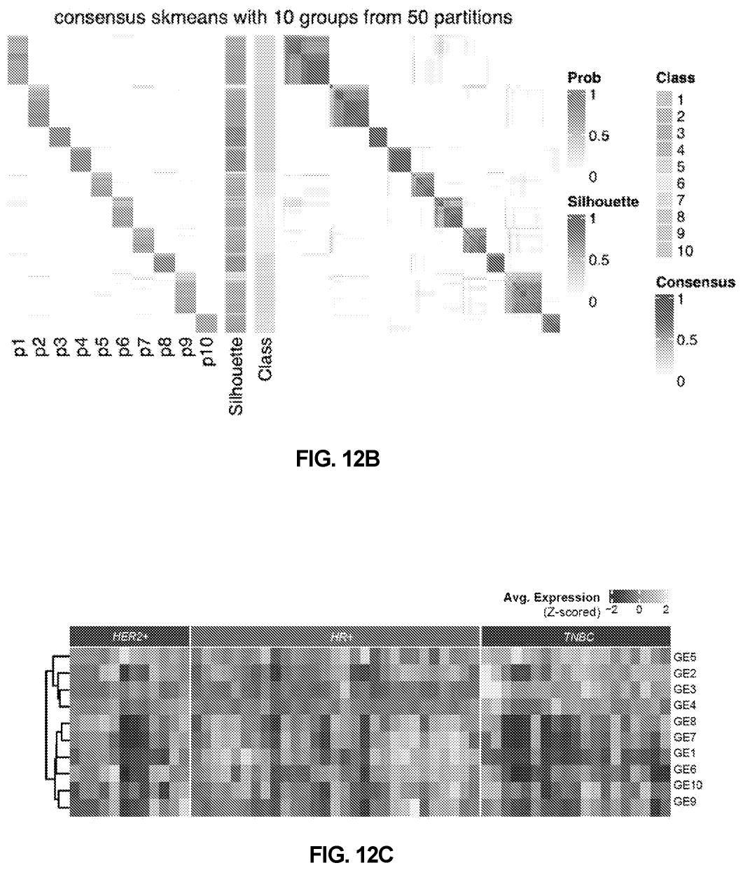

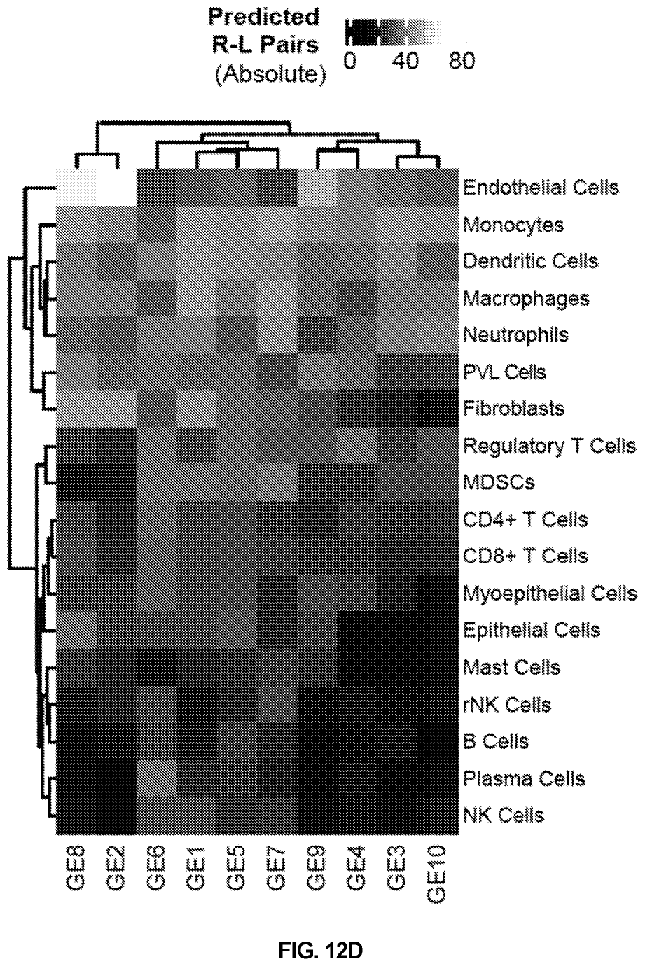

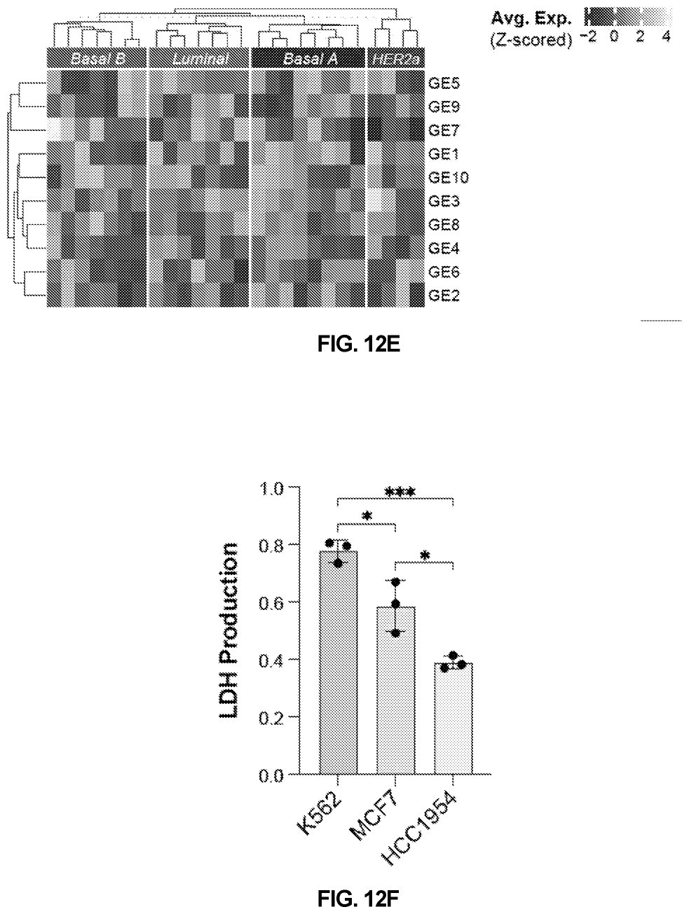

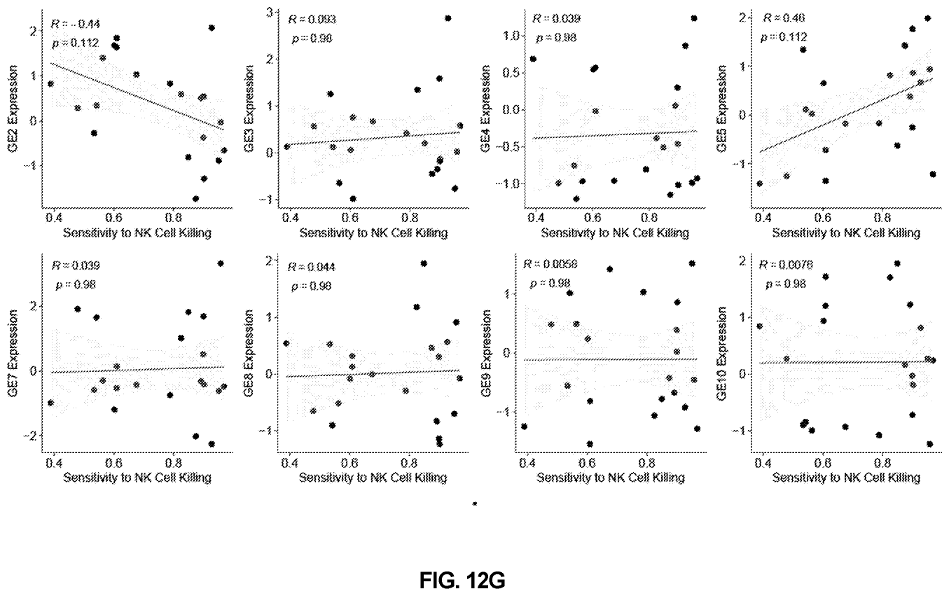

To develop a high-resolution classifier of heterogeneous cancer epithelial cells, we first performed unsupervised clustering on all cancer epithelial cells in the integrated dataset to generate signatures of upregulated genes that capture distinct molecular features of cancer epithelial cell clusters. Next, supervised classification was performed based on expression of 12 clinical therapeutic targets (ESR1, ERBB2, ERBB3, PIK3CA, NTRK1/NTRK2INTRK3, CD274, EGFR, FGFR1/FGFR2/FGFR3/FGFR4, TACSTD2, CDK4/CDK6, AR, NECTIN2) to ensure that clinically relevant associations were captured by upregulated gene signatures (see Methods in Example 10). The motivation for including therapeutic targets was to create classifications grounded in relevant clinical approaches. We additionally supervised classification of all cancer epithelial cells based on molecular subtype to generate upregulated gene signatures that reflect subtype features. Consensus clustering of all generated gene signatures identified 10 unifying groups, which we defined as ‘gene elements’ (GEs) (FIG. 12A-12B). We defined each GE by the top 100 genes that occurred most frequently across gene signatures assigned to the group (Table 7, below; see Methods in Example 10). We scored each cancer epithelial cell by the individual 10 GEs and assigned GE-based cell labels (FIG. 3A; see Methods in Example 10).

TABLE 7
GE Group Gene Signatures

| GE Group | Defining Genes |
|---|---|
| GE1 | AC090498-1, AC105999-2, ADIRF, AGR2, AGR3, ALDH2, ANKRD30A, |
| | ARL6IP1, ARMT1, ATAD2, AZGP1, BATF, BMPR1B, BST2, BTG2, C15ORF48, |
| | CCDC74A, CEBPD, CFD, CLDN4, CLU, COX6C, CPB1, CRIP1, CST3, |
| | CTHRC1, CXCL14, DHRS2, DSCAM-AS1, ELF3, ELP2, ERBB4, ESR1, EVL, |
| | FABP3, FHL2, FKBP5, FSIP1, GJA1, GSTM3, HES1, HSPB1, IFI27, IF16, |
| | IFITM1, IFITM2, IFITM3, IGFBP4, INPP4B, ISG15, JUNB, KCNE4, KCNJ3, |
| | KRT18, KRT19, LDLRAD4, MAGED2, MDK, MESP1, MGP, MGST1, MRPS30, |
| | MRPS30-DT, MS4A7, MT-ATP8, NOVA1, PEG10, PHGR1, PI15, PIP, PLAAT4, |
| | PLAT, PRSS23, PSD3, PVALB, RAMP1, RBP1, RHOBTB3, SCGB3A1, |
| | SCUBE2, SEMA3C, SERPINA1, SH3BGRL, SLC39A6, SLC40A1, SNCG, |
| | STC2, TCEAL4, TCIM, TFF1, TFF3, TIMP1, TMC5, TPM1, TPRG1, VSTM2A, |
| | VTCN1, WFDC2, XBP1, ZFP36L1 |
| GE2 | ALDH3B2, ALOX15B, APOD, AZIN1, B2M, BNIP3, C1orf21, CALD1, CALU, |
| | CAPG, CD24, CD59, CD74, CD99, CDKN2B, CFD, CKB, CLDN3, CLDN4, |
| | CNN3, COL12A1, COX6C, CRIP1, CSRP1, CSRP2, CTNNB1, CTTN, |
| | CYSTM1, DDIT4, DHRS2, DLX5, DSC2, EFHD1, EFNA1, ELF5, ENO1, |
| | FAM229B, FASN, GJA1, GRIK1-AS1, GSTP1, H2AJ, HILPDA, HNRNPH1, |
| | HSPA5, IFI27, IFITM3, IGKC, JPT1, KCNC2, KRT15, KRT23, KRT7, LAPTM4B, |
| | LDHB, LMO4, LTF, MAFB, MAL2, MAOB, MFAP2, MGST1, MRPL15, MT1X, |
| | MUCL1, MYBPC1, NME2, NUPR1, PCSKIN, PFN2, PHGDH, PRSS23, |
| | PSMB3, PTHLH, PTPN1, RAMP1, RAMP3, RBP1, RSU1, S100A10, S100A6, |
| | SCUBE2, SFRP1, SH3BGRL, SLC39A4, SLC40A1, SOX4, STC2, STOM, |
| | TCIM, TFF3, TMSB4X, TTYH1, TUBA1A, UBE2V2, VIM, YBX1, YBX3, YWHAH, |
| | YWHAZ |
| GE3 | A2M, ACTA2, ACTG2, ANGPTL4, ANXA1, APOD, APOE, BGN, C6ORF15, |
| | CALD1, CALML5, CAV1, CAVIN1, CAVIN3, CCL28, CCN2, CD24, CDKN2A, |
| | CHI3L1, COL1A2, COL6A1, COL6A2, COTL1, CRYAB, CSTA, CXCL2, DEFB1, |
| | DEPP1, EFEMP1, FABP5, FBXO32, FDCSP, FGFBP2, FN1, GABRP, GSTP1, |
| | HLA-A, HLA-B, ID1, IFI27, IGFBP3, IGFBP5, IGFBP7, IL32, KLK5, KLK7, |
| | KRT14, KRT15, KRT16, KRT17, KRT5, KRT6A, KRT6B, KRT81, LAMB3, |
| | LCN2, LTF, LY6D, MFAP5, MFGE8, MGP, MIA, MMP7, MT1X, MT2A, MYL9, |
| | MYLK, NDRG1, NDUFA4L2, NFKBIA, NNMT, PDLIM4, PLS3, POSTN, PRNP, |
| | PTN, RARRES1, RCAN1, RGS2, S100A2, S100A4, S100A6, S100A8, S100A9, |
| | SAA1, SAA2, SBSN, SERPING1, SFRP1, SGK1, SLC25A37, SLPI, SPARC, |
| | SPARCL1, TAGLN, THBS1, TPM2, TSHZ2, VIM, ZFP36L2 |
| GE4 | ANLN, ANP32E, ARL6IP1, ASF1B, ASPM, ATAD2, AURKA, BIRC5, BUB1B, |
| | CCNB1, CCNB2, CDC20, CDC6, CDCA3, CDCA8, CDK1, CDKN2A, CDKN3, |
| | CENPA, CENPE, CENPF, CENPK, CENPM, CENPU, CENPW, CIP2A, CKAP2, |
| | CKLF, CKS1B, CKS2, CTHRC1, DEK, DLGAP5, DTYMK, DUT, ECT2, |
| | FAM111A, FAM111B, GGH, GTSE1, H1-2, H1-3, H2AZ1, H2AZ2, H2BC11, |
| | H4C3, HELLS, HMGB1, HMGB2, HMGB3, HMGN2, HMMR, IQGAP3, KIF20B, |
| | KIF23, KIF2C, KNL1, KPNA2, LGALS1, MAD2L1, MKI67, MT2A, MYBL2, |
| | MZT1, NEK2, NUF2, NUSAP1, PBK, PCLAF, PCNA, PLK1, PRC1, PRR11, |
| | PTTG1, RACGAP1, RAD21, RHEB, RNASEH2A, RPL39L, RRM2, SMC4, |
| | SPC25, STMN1, TFDP1, TK1, TMEM106C, TMPO, TOP2A, TPX2, TROAP, |
| | TTK, TUBA1B, TUBA1C, TUBB, TUBB4B, TYMS, UBE2C, UBE2S, UBE2T, |
| | ZWINT |
| GE5 | AIF1, ALOX5AP, ANXA1, APOC1, APOE, AREG, C1ORF162, C1QA, C1QB, |
| | C1QC, CARD16, CCL3, CCL4, CCL5, CD2, CD27, CD37, CD3D, CD3E, CD48, |
| | CD52, CD53, CD69, CD7, CD74, CD83, CELF2, COL1A2, CORO1A, CREM, |
| | CST7, CTSL, CTSW, CXCR4, CYBB, CYTIP, DUSP2, EMP3, FCER1G, FN1, |
| | FYB1, GIMAP7, GMFG, GPR183, GPSM3, GZMA, GZMK, HCST, HLA-DPA1, |
| | HLA-DPB1, HLA-DQA1, HLA-DRA, HLA-DRB1, IGSF6, IL2RG, IL32, IL7R, |
| | ISG15, ITGB2, KLRB1, LAPTM5, LCK, LIMD2, LSP1, LST1, LTB, LY96, LYZ, |
| | MEF2C, MNDA, MS4A6A, MSR1, NKG7, PTPRC, RAC2, RGCC, RGS1, RGS2, |
| | RNASE1, S100A4, S100A6, SEPTIN6, SLC2A3, SMAP2, SOCS1, SPARC, |
| | SPP1, SRGN, STK4, TMSB4X, TNFAIP3, TRAC, TRBC1, TRBC2, TREM2, |
| | TYROBP, VIM, WIPF1, ZEB2, ZNF331 |
| GE6 | ADIRF, ANAPC11, ATP5ME, AZGP1, BLVRB, BST2, CALM1, CCND1, CD9, |
| | CETN2, CISD3, CLDN7, COX6C, CRABP2, CRACR2B, CRIP1, CRIP2, CSTB, |
| | CYB5A, CYBA, CYC1, DBI, DCXR, DSTN, EEF1B2, ELOC, EMP2, FXYD3, |
| | GPX4, GSTM3, H2AJ, H2AZ1, HINT1, HMGB1, HSPE1, IDH2, JPT1, KDELR2, |
| | KRT10, KRT18, KRT19, KRT7, KRT8, LGALS1, LGALS3, LSM3, LSM4, LY6E, |
| | MARCKSL1, MIEN1, MIF, MPC2, MRPL12, MRPL51, MRPS34, MTDH, |
| | MUCL1, NDUFB9, NDUFC2, NME1, PAFAH1B3, PFDN2, PFN1, PIP, POLR2K, |
| | PPDPF, PSMA7, PSMB3, PSME2, RAN, RANBP1, RBIS, REEP5, ROMO1, |
| | RPS26, S100A14, S100A16, SEC61G, SELENOP, SH3BGRL, SLC9A3R1, |
| | SMIM22, SNRPB, SNRPG, SPINT2, SQLE, SRP9, STARD10, TCEAL4, |
| | TMCO1, TMEM14B, TPI1, TPM1, TSPAN13, TUBA1B, TUBB, UQCRQ, XBP1, |
| | YBX1, ZNF706 |
| GE7 | AC093001-1, ADIRF, AGR2, AGR3, ANKRD37, APOD, AQP3, ARC, |
| | AREG, ATF3, AZGP1, BAMBI, BTG1, BTG2, C15ORF48, CALML5, |
| | CCDC74A, CCN1, CD55, CDKN1A, CEBPB, CEBPD, CFD, CLDN3, |
| | CLDN4, CST3, CTD-3252C9-4, CTSK, DHRS2, DNAJB1, DUSP1, |
| | EDN1, EGR1, ELF3, ELOVL2, ESR1, FHL2, FOS, FOSB, GATA3, |
| | GDF15, GRB7, GSTM3, H1-2, HES1, ICAM1, ID2, IER2, IER3, IFITM1, |
| | IGFBP4, IGFBP5, IRF1, JUN, JUNB, KLF4, KLF6, KRT15, KRT18, |
| | LGALS3, MAFB, MAGED2, MGP, NAMPT, NCOA7, NFKBIA, NFKBIZ, |
| | NR4A1, NR4A2, PERP, PLAT, PMAIP1, PRSS23, REL, RHOV, RND1, |
| | S100P, SAT1, SLC39A6, SLC40A1, SOCS3, SOX4, SOX9, STC2, |
| | TACSTD2, TCIM, TFF1, TIMP3, TM4SF1, TNFRSF12A, TSC22D3, |
| | TUBA1A, VASN, VEGFA, VTCN1, XBP1, ZFAND2A, ZFP36, ZFP36L1, |
| | ZFP36L2 |
| GE8 | ADIRF, AFF3, ALCAM, ANKRD30A, ANXA2, AR, ARFGEF3, ASAH1, ATP1B1, |
| | AZGP1, BTG1, CD59, CDK12, CEBPD, CLDN3, CLDN4, CLTC, CLU, CNN3, |
| | CTNNB1, CTNND1, EFHD1, EGR1, ELF3, EPCAM, ERBB2, ESR1, EVL, |
| | FOSB, GATA3, GRB7, H4C3, HES1, HLA-B, HNRNPH1, HSPA1A, HSPA1B, |
| | IGFBP5, INTS6, ITGB1, ITGB6, ITM2B, JUN, KLF6, KRT7, LDLRAD4, LMNA, |
| | LRATD2, MAGED2, MAL2, MARCKS, MT-ND4L, MT2A, MUC1, MYH9, NEAT1, |
| | NFIB, PERP, PKM, PLAT, PMEPA1, PSAP, RAD21, RBP1, RHOB, RUNX1, |
| | S100A10, SAT1, SCARB2, SCD, SDC1, SERHL2, SH3BGRL3, SHISA2, |
| | SLC38A2, SLC39A6, SLC40A1, SOX4, SYTL2, TACSTD2, TCAF1, TCIM, |
| | TFAP2B, TIMP1, TM4SF1, TMC5, TMEM123, TPM1, TRPS1, TSC22D1, |
| | TSPYL1, TUBA1A, VEGFA, WSB1, XIST, YBX1, YBX3, ZFP36L1, ZFP36L2, |
| | ZNF292 |
| GE9 | AC093001-1, ADIRF, AGR2, AGR3, APOD, AQP1, AQP5, AREG, ASCL1, |
| | AZGP1, BMPR1B, C15ORF48, CALML5, CCL28, CD55, CEACAM6, CFD, |
| | CLIC3, CLU, COX6C, CSTB, CTSD, CXCL14, CXCL17, DHRS2, DSCAM-AS1, |
| | DUSP1, ERBB2, FADS2, FAM3D, FHL2, GDF15, GLYATL2, GPX1, GSN, |
| | GSTP1, HDC, HSPB1, IGFBP5, ISG20, ITM2A, KRT23, KRT7, LGALS1, |
| | LGALS3, LY6E, MARCKS, MFGE8, MGP, MS4A7, MT-ATP8, MTCO2P12, |
| | MUC5B, MUCL1, NDRG2, NFKBIZ, NPW, NR4A1, NUDT8, PALMD, |
| | PDZK1IP1, PERP, PHGR1, PIP, PLAT, PRSS21, PSCA, PTHLH, PYDC1, |
| | RGS10, RGS2, RHCG, RP11-53O19-2, S100A1, S100A10, S100A6, S100A7, |
| | S100A8, S100A9, S100P, SAA2, SCGB1D2, SCGB2A1, SCGB2A2, SDC2, |
| | SERHL2, SERPINA1, SLC12A2, SLC18A2, SLPI, SYNM, TACSTD2, TFF1, |
| | TFF3, TM4SF1, TMC5, TSC22D3, TSPAN1, TXNIP, XBP1 |
| GE10 | AGR2, APOD, AREG, AZGP1, B2M, BST2, BTG2, C15ORF48, CCL20, CD74, |
| | CEBPD, CHI3L1, CHI3L2, CP, CRISP3, CSTA, CTSC, CTSD, CTSS, CXCL1, |
| | CXCL17, CYBA, DEFB1, FDCSP, GBP1, GBP2, HLA-A, HLA-B, HLA-C, HLA- |
| | DMA, HLA-DPA1, HLA-DPB1, HLA-DQA1, HLA-DQA2, HLA-DQB1, HLA-DRA, |
| | HLA-DRB1, HLA-DRB5, HLA-E, ID3, IFI16, IFI27, IFI44L, IFI6, IFIT1, IFIT2, |
| | IFIT3, IFITM1, IFITM2, IFITM3, IGFBP7, IL32, IRF1, ISG15, KRT15, KRT19, |
| | KRT5, KRT7, LCN2, LGALS1, LGMN, LTF, LUM, LY6D, LYZ, MAFB, MARCKS, |
| | MGP, MIA, MMP7, MRPS30-DT, MX1, NNMT, PI3, PIGR, RAMP2, RARRES1, |
| | RHCG, RNASE1, RSAD2, S100A8, S100A9, S100P, SAA2, SCGB1D2, |
| | SCGB2A1, SERPING1, SLC39A6, SOD2, SPATS2L, TCIM, TFF1, TFF3, |
| | TMEM45A, TNFAIP6, TNFSF10, TXNIP, WFDC2, XBP1, ZFP36 |

When assessing for molecular subtypes, GES-labeled cells were predominantly assigned to the Basal subtype, while the majority of GE9-labeled cells were assigned to the Her2 subtype (FIG. 38). Cells labeled by GE1 and GE7 were almost exclusively assigned as Luminal A and Luminal B. In contrast, GE5- and GE10-labeled cells were assigned to all molecular subtypes. Next, we used gene set enrichment analysis (FIG. 3C) to identify functional annotations for each GE. This analysis identified shared and distinct functional features for all GEs. GE4 was uniquely enriched for cell cycle and proliferation hallmarks (MK167, PCNA, CDK1). GE2 and GE3 contained hallmark genes of EMT (VIM, ACTA2). GE1, GE6, GE7, and GE9 contained genes associated with estrogen response (ESR1, AREG, TFF3). GE5 and GE10 were enriched for hallmarks of allograft rejection (HLA-DRA, HLA-DRB1), and complement (C1QA/B/C, C1R).

To assess how GE-based cell labels allow us to characterize cancer epithelial cell heterogeneity within a tumor sample, we applied our GEs to the integrated dataset to deconstruct each individual patient tumor into the 10 GEs (FIG. 12C). Notably, GE-based heterogeneity was not constrained by clinical or molecular subtype. This again confirms that significant cancer epithelial cell ITTH exists even within cells from a tumor labeled by a single clinical or molecular subtype. Overall, we generated 10 GEs to characterize cancer epithelial cell ITTH and deconstruct a heterogeneous tumor into its diverse cellular phenotypes.

### Example 5—Gene Elements Predict Individual Patient Predominant Immune Response

To examine how cancer epithelial cell ITTH influences immune interactions in the TME, we generated a decoder matrix of predicted GE-immune interaction strength. GE-immune interaction strength is determined based on the scaled number of predicted receptor-ligand pairings between GEs and immune cells (FIG. 3D; FIG. 12D; see Methods in Example 10).

To experimentally validate the decoder matrix, we tested these predictions with human breast cancer cell lines. In the decoder matrix, cancer epithelial cells labeled by GE1 and GE6 were predicted to highly interact with NK cells (GE1 and GE6 have the highest scaled number of curated receptor-ligand pairings). We applied the GEs to human breast cancer cell lines from the Cancer Cell Line Encyclopedia to quantify GE expression across cell lines (FIG. 12E). Given that GE1 and GE6 have the greatest predicted interaction strength with NK cells (FIG. 3D), we hypothesized that expression of these GEs will have a significant influence on NK cell function (i.e., sensitivity or resistance of cancer cell lines to NK cell killing). To test this, we selected breast cancer cell lines with differing expression of GE1 and GE6. HCC1954 had increased expression of GE1 and GE6, while MCF7 had decreased expression of GE1 and GE6. Using these selected cell lines, we assessed the relationship between GE1 and GE6 expression and sensitivity to NK cell killing. We co-cultured HCC1954 (GE1 and GE6 high) and MCF7 (GE1 and GE6 low) with NK-92, a human NK cell line. As hypothesized, GE1 and GE6 expression had a statistically significant impact on NK cell function. NK cell cytotoxicity against HCC1954 at 12 hours was significantly reduced (p<0.05) compared to NK cell cytotoxicity against MCF7 (FIG. 12F). This finding suggests that GE1 and GE6 confer resistance to NK cell cytotoxicity. Next, to expand on these experimental findings, we used a paper by Sheffer et al. which reports experimental sensitivity or resistance of 26 breast cancer cell lines to NK cell cytotoxicity (see Methods in Example 10) (Sheffer M et al. Nature Genetics. 2021; 53 (8): 1196-206; Barretina J et al. Nature. 2012; 483 (7391): 603-7). Increased GE1 and GE6 expression was significantly correlated with increased resistance to NK cell killing (R=−0.59, p<0.05 for GE1; R=−0.562, p<0.05 for GE6) (FIG. 3E), consistent with the decoder matrix and our experimental findings. Other GEs with fewer predicted NK cell interactions in the decoder matrix did not have statistically meaningful correlations with sensitivity to NK cells (FIG. 12G). To investigate interactions that contribute to these phenotypes, we assessed predicted receptor-ligand pairs between cells that highly express GE and NK cells (FIG. 3F). We observed that GE1- and GE6-labeled cells were predicted to have receptor-ligand pairs that have been characterized as inactivators of NK cell activity (e.g., NECTIN2 TIGIT, THBSLCD47, CD320 TGFRB2). These functional studies validate two of the predictions made by the decoder matrix, by showing that GE1 and GE6 are predictive of significant resistance to NK cell killing for breast cancer cell lines.

Overall, this decoder matrix provides a blueprint for quantifying the degree of interactions between each GE and different immune cell types. Moreover, this decoder matrix curates key activating and inhibitory receptors that can be used to infer how GE-immune interactions affect immune cell behavior.

### Example 6—Spatial Mapping of Gene Elements Reflects Predicted Immune Interactions

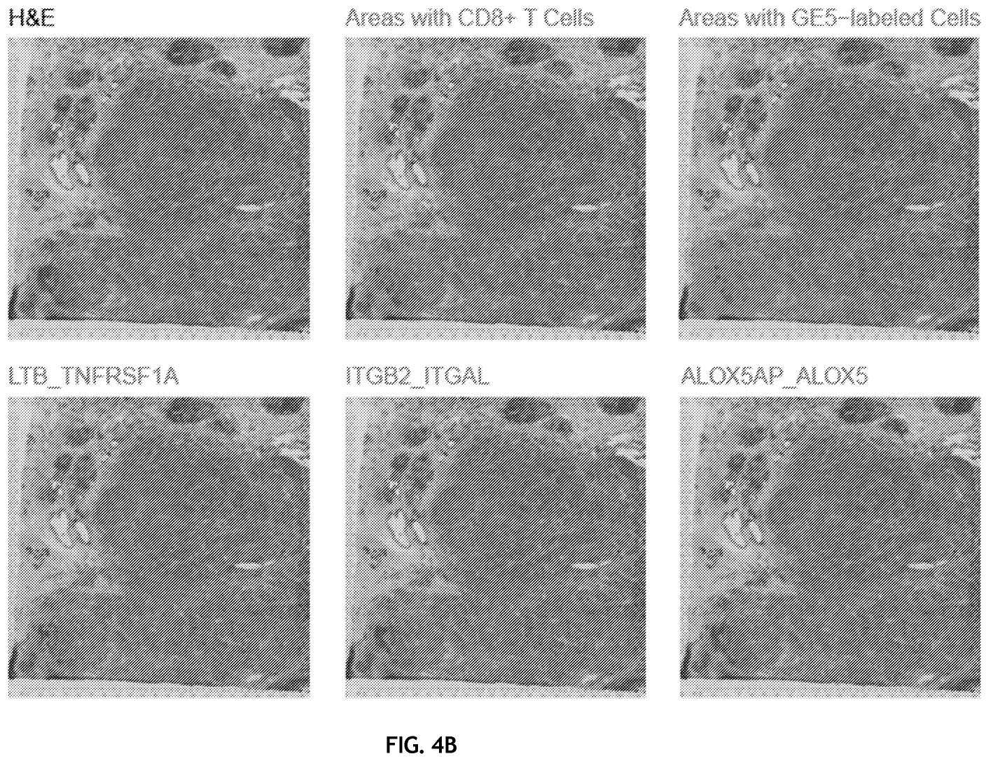

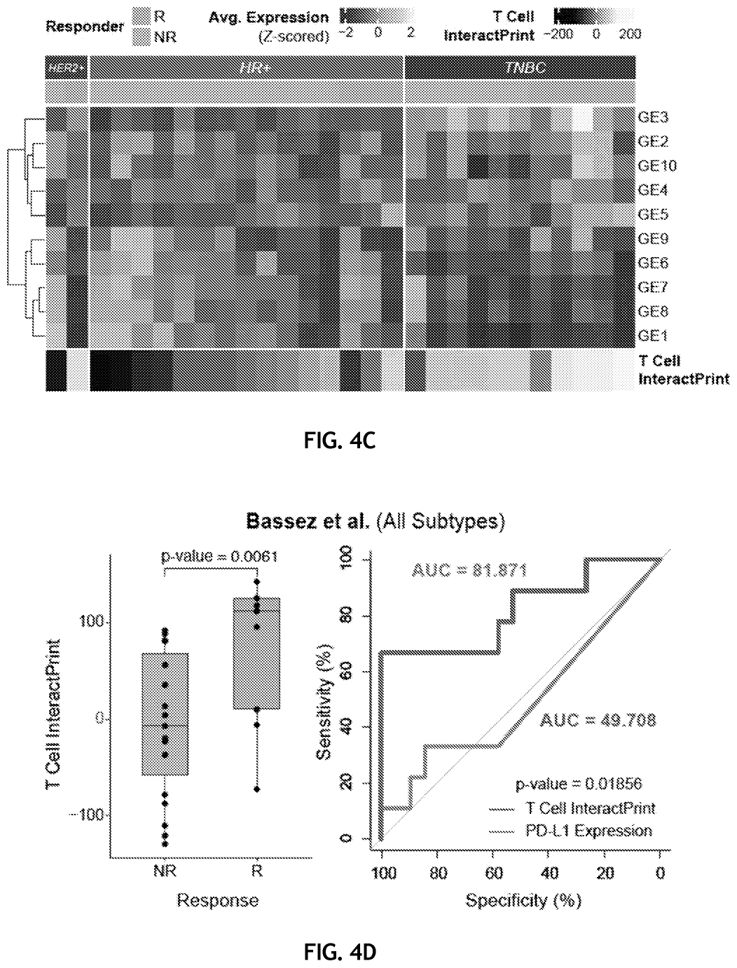

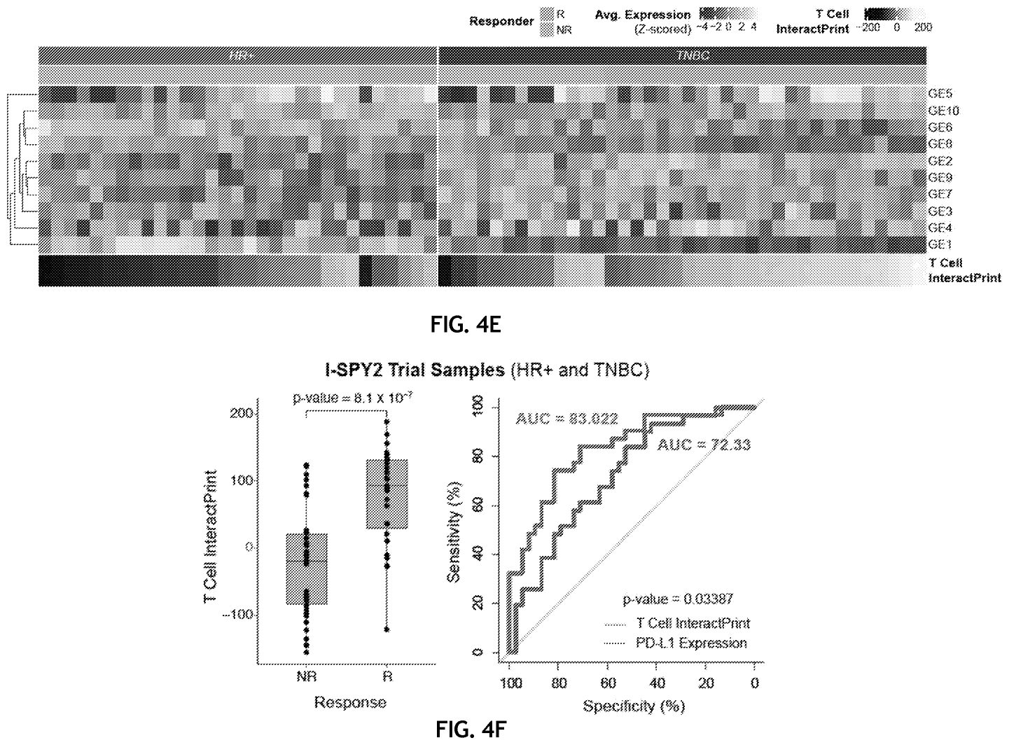

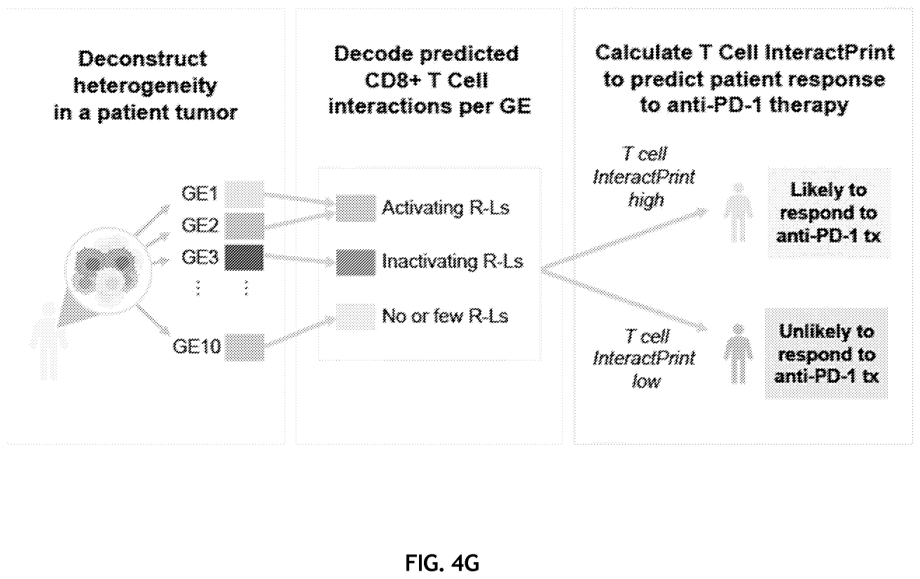

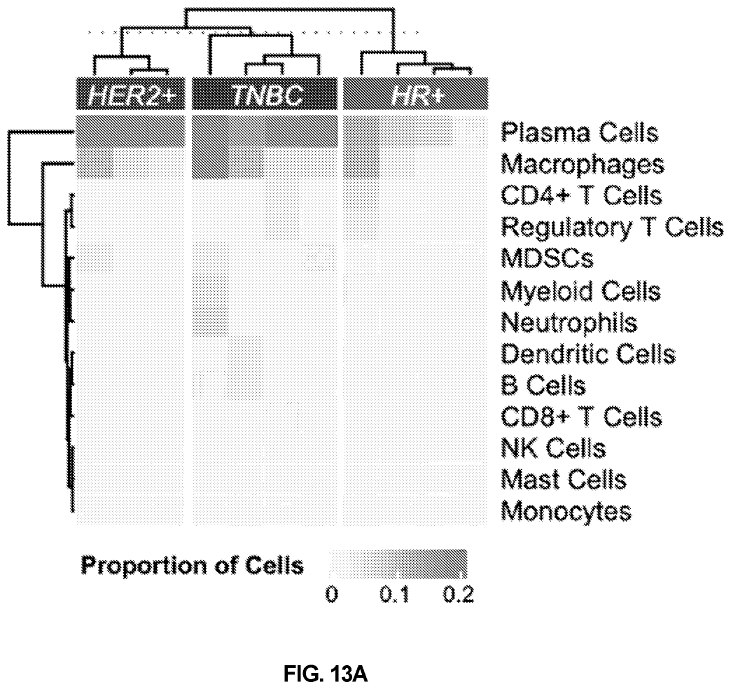

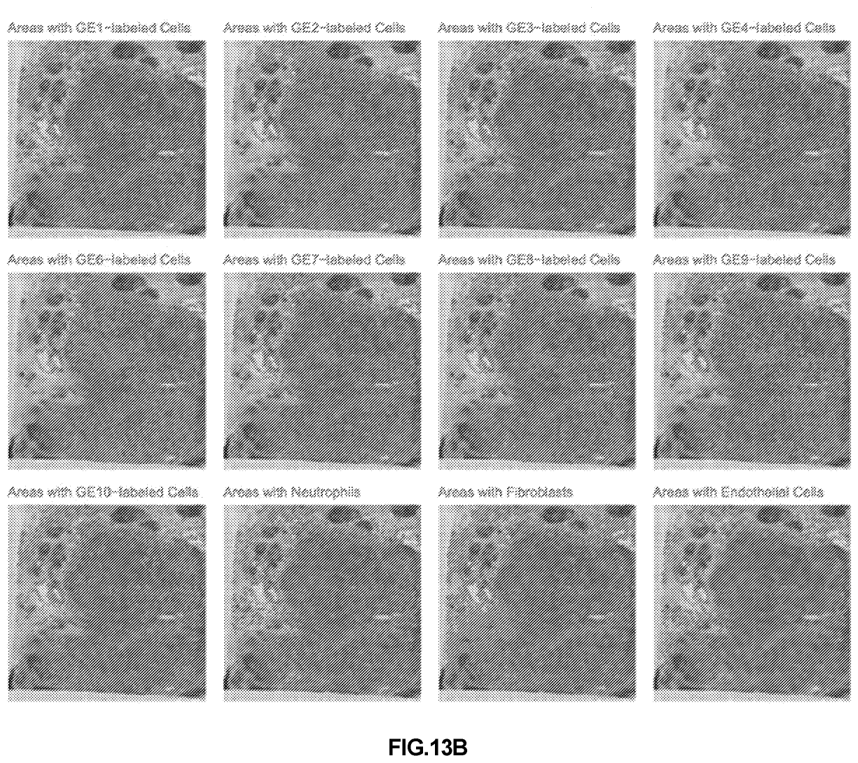

To validate the predicted interactions curated by the decoder matrix, we used a spatial transcriptomics dataset containing published data from 10× Genomics and from Wu S Z et al. Nature Genetics. 2021; 53 (9): 1334-47. We first deconvoluted the underlying composition of cell types through integration of the spatial transcriptome data with the integrated dataset (see Methods in Example 10). Because T cell infiltration was relatively high across spatial transcriptomics samples (FIG. 13A), we chose to explore T cell interactions using this dataset. To do so, we applied the 10 GEs to each sample in the dataset. Using the decoder matrix, we inferred which GE-labeled cells interact with T cells and which ones do not. Thus, we hypothesized that these GE-labeled cells and CD8+ T cells would be spatially organized in breast tumors. To test this, we examined the co-expression of the GEs and the presence of neighboring CD8+ T cells. Notably, GE5 expression demonstrated positive correlations with CD8+ T cells in all samples (mean R=0.33; all p<0.0001) (FIG. 4A). In one representative image, we determined the co-localization of CD8+ T cells with GE5 expression (FIG. 4B). For areas with high presence of CD8+ T cells, we observed increased colocalization of select curated receptor-ligand pairs (ITGB2 ITGAL, LTB TNFRSF1A, ALOX5AP ALOX5) (FIG. 4B). As expected, GEs with limited predicted interactions did not consistently co-localize with CD8+ T cells (FIG. 13B).

### Example 7—InteractPrint: A Weighted Score to Predict the Predominant Tumor Interacting Immune Cell for an Individual Patient Tumor

We then hypothesized that the GE-immune interaction decoder matrix could be applied to individual tumor tissues. To account for how cancer epithelial cell ITTH within a tumor influences immune cell interactions, we developed InteractPrint. InteractPrint reflects interactions between the predominant tumor-responsive immune cells from the decoder matrix and cancer cells which highly express each GE, weighted by the GE composition of an individual patient tumor. This approach permits real-world application of InteractPrint since it accounts for heterogeneity of GEs within a tumor.

### Example 8—InteractPrint Predicts Anti-PD-1 Therapeutic Response

We then sought to use InteractPrint to characterize the predominant immune response within patients for therapeutically targeted immune cells. Because current immune checkpoint inhibitors (ICI) target CD8+ T cell-driven cancers, we developed T cell InteractPrint to predict who might respond to ICI. For the comparator, average PD-L1 expression on cancer epithelial cells was selected, as PD-L1 remains the main biomarker used clinically to determine who should receive ICI for many solid tumors, including patients with recurrent unresectable or metastatic TNBC (Pardoll D M. Nat Rev Cancer. 2012; 12 (4): 252-64): Network NCC. Breast Cancer (v4.2022) Accessed Oct. 1, 2022, nccn.org/professionals/physician_gls/pdf/breast.pdf).

We applied our approach to a separate scRNA-seq dataset published by Bassez et al., which contains tumor biopsies from breast cancer patients pre- and post-anti-PD-1 therapy (Bassez A et al. Nature Medicine. 2021; 27 (5): 820-32) (FIG. 12C-12D). Deconstruction of each individual patient tumor into the 10 GEs revealed considerable cancer epithelial cell ITTH prior to anti-PD-1 treatment (FIG. 4C), similar to what was observed in the integrated dataset (FIG. 12C). To assess the capacity of the T cell InteractPrint to predict responders to anti-PD-1 therapy, we derived receiver operating characteristic (ROC) curves in this dataset (FIG. 4D). Across clinical subtypes of breast cancer, the T cell InteractPrint demonstrated an area under the curve (AUC) of 81.87% (p=0.0061) in predicting response to anti-PD-1 therapy, inferred from T cell clonotype expansion (Sheffer M et al. Nature Genetics. 2021; 53 (8): 1196-206). This was a significant improvement (p=0.019) over average PD-L1 expression on cancer epithelial cells, the current clinical biomarker to predict patients who will respond to anti-PD-1 therapy in breast cancer, which had an AUC of 49.71% (p>0.05).

Next, we applied our predictor to a separate validation dataset containing results from the I-SPY2 trial. I-SPY2 is an ongoing, multicenter, open-label, adaptively randomized phase 2 multicenter trial of neoadjuvant chemotherapy for early-stage breast cancer at high risk of recurrence (Nanda R et al. JAMA Oncology. 2020; 6 (5): 676-84). In this trial, patients with breast cancer received anti-PD-1 therapy (same as patients from Bassez et al.) combined with paclitaxel. We applied the 10 GEs to microarray data from pre-treatment tumor samples from the I-SPY2 trial and observed levels of heterogeneity that were comparable to those described in the scRNA-seq datasets (FIG. 4E). In the I-SPY2 trial dataset, T cell InteractPrint (AUC=83.02%; p=8.1×10⁻⁷) demonstrated significant improvement (p=0.034) over average PD-L1 expression on cancer epithelial cells (AUC=72.33%; p=0.001) in predicting response to anti-PD-1 therapy (FIG. 4F).

Across two trials, T cell InteractPrint demonstrated significant improvement over PD-L1 at predicting response to anti-PD-1 therapy. This highlights the ability of T cell InteractPrint to decode how cancer epithelial cell ITTH impacts CD8+ T cell response for each individual patient.

### Example 9: Discussion of Examples 1 to 8

In this study, we present a novel atlas resource that integrates scRNA-seq data of 236,363 cells that represent the breast tumor microenvironment (TME). This new resource enables high-resolution characterization of rare immune cell and cancer epithelial cell heterogeneity and demonstrates how heterogeneity influences immune cell interactions which have not been previously evaluated.

First, the statistical power of this integrated dataset is leveraged to demonstrate how NK cells, a population of rare immune cells that have not been classified in the breast TME, can be further studied. Six subsets of NK cells were identified consisting of activated and cytotoxic, exhausted, and reprogrammed NK cells. Identification of rNK cells in most but not all samples (i.e., 72% of samples) provides a subtype-independent approach to identify patients who may benefit from rNK cell-directed therapies. These findings add to the growing body of literature on distinct NK cell subsets and phenotypes. In particular, the gene expression profile of the cytotoxic NK-2 subset aligns with CD56^{dim} subsets previously identified in bone marrow by Crinier et al. (Crinier A et al. Cellular & Molecular Immunology. 2021; 18 (5): 1290-304) and Yang et al. (Yang C et al. Nature Communications. 2019; 10 (1): 3931), in peripheral blood by Smith et al. (Smith S L et al. Blood Advances. 2020; 4 (7): 1388-406), and in human melanoma metastases by de Andrade et al. (de Andrade L F et al. JCI Insight. 2019; 4 (23)). The NK-0 subset closely resembles previously described ‘memory-like’ NK cells derived from bone marrow by Crinier et al. and have been described after CMV or tumor exposure (Crinier A et al. Cellular & Molecular Immunology. 2021; 18 (5): 1290-304). The description of NK-4 aligns with prior observations of “inflamed” IFN-responding NK cells in the bone marrow by Yang et al. (Yang C et al. Nature Communications. 2019; 10 (1): 3931) and in peripheral blood by Smith et al. (Smith S L et al. Blood Advances. 2020; 4 (7): 1388-406). NK-3 demonstrated features consistent with prior studies of tissue-resident NK cells derived from bone marrow by Yang et al. (Yang C et al. Nature Communications. 2019; 10 (1): 3931) and from melanoma metastases by de Andrade et al. (de Andrade L F et al. JCI Insight. 2019; 4 (23)). The unique transcriptional profile of the NK-5 subset has been previously described as exhausted (Chan C J et al. Nature Immunology. 2014; 15 (5): 431-8; Braud V M et al. Nature. 1998; 391 (6669): 795-9). Lastly, similar expression profiles (e.g., upregulated NR4A family, DUSP1. FOS, JUN) to the reprogrammed NK-1 subset have been described in peripheral blood by Smith et al. (Smith S L et al. Blood Advances. 2020; 4 (7): 1388-406), in bone marrow by Yang et al. (Yang C et al. Nature Communications. 2019; 10 (1): 3931), and in human head and neck cancers by Moreno-Nieves et al. (Moreno-Nieves U Y et al. Proceedings of the National Academy of Sciences. 2021; 118 (28): e2101169118), as well as in our prior studies on metastasis-promoting NK cells derived from ex vivo and mouse model (Chan I S et al. Journal of Cell Biology. 2020; 219 (9)). Data in these examples provide the first evidence to identify six subsets of NK cells in human primary breast tumors, which can now be quantified and measured in response to prospective therapeutics.

Through this analysis, it was observed that NK cell heterogeneity is associated with breast cancer clinical subtypes. These clinical subtypes are well known to harbor substantial heterogeneity (Polyak K et al. J Clin Invest. 2011; 121 (10): 3786-8; Turashvili G et al. Front Med (Lausanne). 2017; 4:227; Schettini F et al. npj Breast Cancer. 2021; 7 (1)). This led us to use this novel resource to further understand clinically relevant heterogeneity within the breast TME and cancer epithelium at resolutions higher than previously studied. At the single-cell resolution, we quantified the heterogeneity of single-gene expression (i.e., ERBB2 and TACSTD2) across tumors and found that the majority of samples across all breast cancer subtypes expressed ERBB2 and TACSTD2. The new class of antibody-drug conjugates targeting these proteins have recently demonstrated efficacy across breast cancer subtypes. For HER2/ERBB2, high concordance between proteomic HER2 status and ERBB2 mRNA expression has been reported in the literature (Wu N C et al. Breast Cancer Res Treat. 2018; 172 (2): 327-38; Denkert C et al. Breast Cancer Res. 2013; 15 (1): R11; Wang Z et al. J Mol Diagn. 2013; 15 (2): 210-9; Vassilakopoulou M et al. PLoS One. 2014; 9 (6): e99131), and we corroborate these findings in the integrated dataset (FIG. 10D). Similarly, for TROP2/TACSTD2, concordance between proteomic TROP2 and TACSTD2 mRNA expression has been reported in various solid tumors, including breast (Coates J T et al. Cancer Discov. 2021; 11 (10): 2436-45; Zhao W et al. Oncol Rep. 2018; 40 (2): 759-66; Chou J et al. Eur Urol Oncol. 2022; 5 (6): 714-8; Ohmachi T et al. Clin Cancer Res. 2006; 12 (10): 3057-63; Bignotti E et al. Eur J Cancer. 2010; 46 (5): 944-53; Stepan L P et al. J Histochem Cytochem. 2011; 59 (7): 701-10). Further, examining genes that are positively correlated with ERBB2 and TACSTD2 uncovers other potential clinical targets that can synergize with current anti-HER2 and anti-TROP2 therapies and provides rationale for novel combination approaches. Then, we characterized cancer epithelial cell heterogeneity by using unsupervised and supervised molecular clustering to determine the how each cell relates to established breast cancer clinical and molecular subtypes. While discrepancies between clinical and molecular subtyping have been well documented, we provide an approach to defining cancer epithelial cell heterogeneity at the single-cell level by using 10 GEs. This approach enables high-resolution characterization of cancer epithelial cell ITTH and deconstruction of a heterogeneous tumor into its diverse epithelial phenotypes.

To further demonstrate how this new resource facilitates analysis of the breast TME, we then use information from the 10 GEs to identify how cancer epithelial cell heterogeneity influences interactions with immune populations. Current ICI biomarker approaches mainly focus on the expression of single targets, resulting in an incomplete characterization of the TME complexity. Our approach for T cell InteractPrint score calculates cancer epithelial cell heterogeneity within a tumor sample and the number of predicted interactions between heterogeneous cancer epithelial cells and CD8+ T cells (FIG. 4G). This captures how heterogenous expression of GEs shifts the predicted strength of T cell interactions for an individual patient's tumor. Across two trials and all subtypes of breast cancer, T cell InteractPrint predicted response to T cell immune checkpoint inhibition. This finding is significant, because anti-PD-1 therapy is not effective in HR+ disease (Rugo H S et al. Clin Cancer Res. 2018; 24 (12): 2804-11), and has limited efficacy in TNBC disease (Kwa M J et al. Cancer. 2018; 124 (10): 2086-103) when compared to the response seen in other solid tumors (Gandhi L et al. N Engl J Med. 2018; 378 (22): 2078-92; Garon E B et al. N Engl J Med. 2015; 372 (21): 2018-28; Schachter J et al. Lancet. 2017; 390 (10105): 1853-62; Bellmunt J et al. N Engl J Med. 2017; 376 (11): 1015-26). The development of InteractPrint from this resource serves as another example of how this new resource can be used to uncover new biology that, once validated, could inform response to ICI in breast cancer.

A limitation of our study is that we compared InteractPrint to PD-L1 by transcriptomic expression in early-stage breast cancer trials. In breast cancer, the approval of PD-L1 is assessed by IHC in the setting of recurrent unresectable or metastatic TNBC disease. However, there is still a need for improved patient selection given the multiple adverse events associated with ICIs. Future prospective studies are warranted to compare T cell InteractPrint and PD-L1 gene and protein expression, along with other biomarkers, to predict response to ICI.

The breast TME is a complex ecosystem that encompasses diverse cell phenotypes, heterogenous interactions among cells, and varied expression of clinically targetable features. The development of this new resource and examples of its utility uncovered information about NK cells and how heterogenous cancer epithelial cells and their predicted immune interactions can predict immune checkpoint therapy responses. Future use of this resource is likely to yield additional impactful findings.

### Example 10: Materials and Methods

### Processing of Single-Cell RNA-Seq Datasets

119 primary breast tumor samples across 8 publicly available datasets from 88 untreated female patients ranging from 32 to 90 years of age encompassing all clinical subtypes were obtained. As the collected datasets were not aligned to the same genome, all gene names were converted to the official gene alias as delineated by the HUGO Gene Nomenclature Committee (HGNC) using the limma (v3.50.1) and org.Hs.eg.db (v3.14.0) packages (51,52). Each dataset was then filtered based on percent mitochondrial transcripts, percent hemoglobin genes, number of RNA molecules, and number of features. In brief, cells lower than the 5th percentile and greater than the 95th percentile of each metric were removed, as well as those cells with greater than 15% mitochondrial content.

To avoid confounding clustering and gene expression analyses, the DoubletFinder (v2.0.3) package was used to identify and remove doublets from the dataset (53). Doublet rates were estimated based on given rates from the original technology used and the cell loadings provided by the original studies.

### Integration of Primary Breast Cancer Datasets by Seurat

The 119 untreated primary samples were integrated via reference-based integration using Seurat (v4.1.0) to remove any technology-driven batch effects (Hao Y et al. Cell. 2021; 184 (13): 3573-87.e29). To prevent over-correction of the data, SCTransform (v0.3.2.9008) was used rather than the standard Seurat normalization scheme (Hafemeister C et al. Genome Biology. 2019; 20 (1): 296). This was done according to the developers' vignette (satijalab.org/seurat/articles/integration_large_datasets.html). The 10× datasets were chosen as the reference and the rann method was chosen for FindNeighbors. Success of batch effect correction was determined by visually inspecting the top two principal components and ensuring that no single technology, cohort, or subtype was driving any clusters (FIGS. 5D-5L, 6A-6I).

### Cell Type Annotation and Clustering

First, general cell types were identified using canonical and literature-derived cell markers (Table 3, above). After these were determined, three methods were used to refine each cell's identity. The first utilized cluster-level annotations via the UCell (v1.99.1) package; the second labeled cells based on thresholds based on the number of markers, and then clustered and calculated the average expression of those markers to refine the cell identities; and the third took highest average expression of select markers. The annotation with highest agreement across these methods was selected as the cell type. If all methods disagreed, then the overall cluster labeling was chosen as the annotation for that cell.

For the cluster-level method, all cell markers were aggregated into a single score using the AddModuleScore_UCell function from the UCell (v1.99.1) package and visualized using FeaturePlot from the Seurat (v4.1.0) package. The clusters that had the highest overall for a given cell type were labeled as that type, isolated and re-integrated to account for batch effects. Subtype-specific cell markers were applied (e.g., CD4 for CD4+ T cells).

For the second method, cell type annotations were identified as a given type based on the number of markers that had non-zero expression for a given cell. In brief, epithelial cells were labeled as such if they had two epithelial markers or if they had at least one of the following markers: EPCAM, KRT8, KRT18, and KRT19. Specific immune types were labeled as such if they either had at least two markers of that type and no other type, PTPRC and at least one marker of that type and no other, or at least three markers for that type and at most one marker of a different immune type. Stromal cell types could either have only cell-type-specific markers or at least three cell-type-specific markers and at most one endothelial marker. Finally, endothelial cells could have either only endothelial markers or at least three endothelial markers and at most one marker associated with a stromal cell type.

Lastly, we examined log-normalized expression values of the selected markers for each cell. A cell was assigned to the cell type that had the highest average expression for their markers across all features. T and myeloid subsets were identified in the same manner once the cells were identified as T cells or myeloid cells respectively.

The final cell call was determined based on the highest consensus or defaulted to the larger cluster's identity. Of the 116,346 cells which had original source annotations, 93% had concordant annotations between the original source and our analysis.

### Identification of Cancer Epithelial Cell Populations

Epithelial cells were re-clustered and re-integrated to account for batch effects (FIGS. 10A-10C). Copy number variant (CNV) profile analysis was used for cancer (malignant) versus normal (non-malignant) assignments. The CNV signal for individual cells was estimated using inferCNV (v.0.99.7) with a 100-gene sliding window; genes with mean count less than 0.1 across all cells were filtered out, and the signal was denoised using a dynamic threshold of 1.3 s.d. from the mean (83). Non-T cell immune cells were used for the reference cell profiles. Epithelial cells were classified as normal (non-malignant), cancer (malignant), or unassigned using a previously described method (84). Briefly, inferred changes at each genomic locus were scaled (between −1 and +1) and the mean of the squares of these values was used to define a CNV signal for each cell. For each sample, an average CNV profile was created, and each cell in the sample was then correlated to this profile for the CNV correlation score. Epithelial cells were classified cancer vs. normal based on CNV signal and CNV correlation, with thresholds of 0.4 for CNV correlation and 0.02 for CNV signal (FIGS. 7A-7B). This assigned 75,883 cancer, 3,524 normal, and 4,997 unassigned epithelial cells.

Within cancer epithelial cells, ERBB2-positive and TACSTD2-positive cells were chosen due to clinical relevance. ERBB2 and TACSTD2 expression levels are calculated using UCell (v1.99.1). ERBB2^{Hi} cancer epithelial cells were defined by positive ERRB2 expression above the 97.5^{th} percentile, ERBB2^{Med} cells were defined by positive ERBB2 expression at or below the 97.5^{th} percentile, and ERBB2^{Lo} cells were defined by zero ERBB2 expression. TACSTD2^{Hi} cells were defined by positive TACSTD2 expression above the 95th percentile, TACSTD2Med cells were defined by positive TACSTD2 expression at or below the 95th percentile, and TACSTD2^{Lo} cells were defined by zero TACSTD2 expression.

FindMarkers in Seurat (v4.1.0) and MAST (v1.20.0) were then used to identify differentially expressed genes in each cluster (>5 cells per cluster, only test genes detected in >20% of cells in a cluster). Expression levels of clinically actionable targets for each subset of cells was estimated by AverageExpression by Seurat (v4.1.0). For visualization of differentially expressed genes (FIGS. 10H-10K), the log₂ fold change cutoff was increased to 1.5 and a false discovery rate cutoff was set to be equal to 0.05. Gene set enrichment analysis across the ERBB2 and TACSTD2 groups was performed using clusterProfiler (v4.2.2) and the Hallmark gene set collection from msigdbr (v7.5.1) (58,59) using an additional cutoff of the absolute difference in percent expression between the pairwise populations >0.1. Only genes with a log₂ fold change >0 were considered. For exploring associations with clinical features, linear regression and Pearson correlations were calculated between the proportion of ERBB2-positive or TACSTD2-positive cells per sample and the age or nodal status and these analyses were stratified by subtype in FIG. 11A-11C. We additionally explored associations between % TACSTD2+ cells and nodal status in each cohort and then combined the results using Fisher's combined probability test, which was found to not be statistically significant (FIG. 11C; Fisher's combined probability X=11.227, p=0.08). In contrast, for the integrated dataset, there was a statistically significant association between % TACSTD2+ cells per sample and nodal across all samples with nodal status clinical data (p<0.05, n=38).

### Identification of Natural Killer Cell Subsets

NK subpopulations were found through the isolation and re-integration of the larger NK cluster gain to ensure the removal of batch effects (FIG. 8A). Given the higher dimensionality of the dataset containing only NK cells (i.e., number of features>>number of NK cells), the Manhattan distance metric was used. FindMarkers in Seurat (v4.1.0) and MAST (v1.20.0) were used to identify differentially expressed genes for each cluster (Bonferroni adjusted p value<0.05. For marker gene selection, the absolute log₂ fold change cutoff was increased to a baseline of 0.56. Thresholds used to select the highest marker genes for each NK subset are included in Table 6 below. Marker genes for each NK cell subset are included in Table 4, above.

TABLE 6

| NK subset | Threshold for Upregulated Genes | Threshold for Downregulated Genes |
|---|---|---|
| NK-0 | avg_log2FC >0, pct. 1 >=0.2 | avg_log2FC <0 and abs(avg_log2FC) >=0.7, |
| | | difference in percent expression |
| | | between NK-0 and every other subset >= to 0.1 |
| NK-1 | avg_log2FC >0, difference in | avg_log2FC <0 and abs(avg_log2FC) >=0.65, |
| | percent expression between | difference in percent expression |
| | NK-1 and every other subset >=0.218 | between NK-1 and every other subset >=0.1 |
| NK-2 | avg_log2FC >0 and abs(avg_log2FC) >0.75, | avg_log2FC <0 and abs(avg_log2FC) >=0.75, |
| | difference in percent expression | difference in percent expression |
| | between NK-2 and every other subset >0.21 | between NK-2 and every other subset >=0.1 |
| NK-3 | avg_log2FC >0 and abs(avg_log2FC) >0.8, | avg_log2FC <0 and abs(avg_log2FC) >0.8, |
| | difference in percent expression | difference in percent expression |
| | between NK-3 and every other subset >0.1 | between NK-3 and every other subset >0.15 |
| NK-4 | avg_log2FC >0, difference in percent | Not provided |
| | expression between NK-4 and every | |
| | other subset >0.28 | |
| NK-5 | avg_log2FC >0, difference in percent | avg_log2FC <0 |
| | expression between NK-5 and every | |
| | other subset >0.25 | |

To identify human reprogrammed tumor-promoting NK cells, we first developed a 99 gene signature that based on genes upregulated in tumor-exposed NK cells as compared to healthy NK cells in MMTV-PyMT and WT FVB/n mice as previously described. In an earlier study, primary healthy and tumor-exposed NK cells were isolated and total RNA was extracted and sequenced using Illumina NextSeq 500. Bulk RNA-seq paired-end reads were then aligned and mapped using hisat2 and HTSeq respectively, and DESeq2 was used for differential gene expression analysis between these two populations.

For application in the current study, the mouse genes were converted into their human aliases using the BioMart (v2.50.0) package. Because the mouse strain used in the previous study (MMTV-PyMT) most closely resembles the luminal A/luminal B and basal subtypes, these same subtypes were analyzed for rNK presence. NK cells within each subtype that were in the 75th percentile for this 90 gene signature were labeled as “reprogrammed”. Removing any duplicates resulting from HR+ cells being included in the luminal A and luminal B groups led to 841 total rNK cells in the dataset.

Gene set enrichment analysis across the NK subsets was performed using clusterProfiler (v4.2.2) and the Hallmark gene set collection from msigdbr (v7.5.1) (58,59). Only genes with a log₂ fold change >0 were considered. Samples with fewer than 10 NK cells were omitted. For visualization of differentially expressed genes, the log₂ fold change cutoff was increased to 1.5 and a false discovery rate cutoff was set to be equal to 0.05. To examine expression of the INK signature within the NK cell subsets and across the clinical subtypes, the Kruskal-Wallis test and a pairwise post-hoc Dunn test was performed when appropriate. For the similarity analysis of rNK cells, the expression matrix was reduced to the reprogrammed signature, and the Pearson correlation coefficient was calculated for all pairwise combinations of rNK cells with rNK cells and for rNK cells with non-rNK cells. These analyses were also stratified by age to ensure that age was not a confounder (FIG. 9D-9F).

### Molecular Subtyping of Samples Using SCSubtype

To identify the molecular breast cancer subtypes within the integrated dataset, we used the SC50 Subtype gene signature described in Wu et al. (8).

In brief, the mean read counts for each signature were determined and the highest mean was assigned as the subtype for that cell. To identify the molecular subtype of each sample, we determined the number of cells classified under each SC50 subtype, and then picked the subtype with the highest number of cells to be the tumor molecular subtype for that sample following the method of Wu et al. (8).

### Gene Signature Analysis of Cancer Epithelial Cell Heterogeneity

For each individual tumor sample with more than 50 epithelial cells, heterogeneity was assessed using ROGUE, an entropy-based statistic that enables accurate and sensitive assessment of cluster purity (48). To identify samples with discordance between heterogeneity as characterized by the ROGUE score versus by molecular subtype, we calculated the difference between the normalized ROGUE score and the highest percentage of cells of a single subtype. Samples with difference over 50% were determined to be discordant.

Unsupervised and supervised clustering of cancer epithelial cells for each individual tumor sample with more than 50 epithelial cells was performed. We generated an exhaustive collection of gene signatures that reflect molecular features of different cancer epithelial cells.

For unsupervised clustering on the integrated dataset, all cancer epithelial cells were clustered at 15 resolutions (0.01, 0.05, 0.08, 0.1, 0.2, 0.3, 0.4, 0.5, 0.6, 0.7, 1.0, 1.3, 1.6, 1.8, 2.0) utilizing Seurat (v4.1.0) (54). FindMarkers in Seurat (v4.1.0) and MAST (v1.20.0) were then used to identify differentially expressed genes in each cluster (>5 cells per cluster, only test genes with >25% difference in the fraction of detection between the clusters, log₂FC>0.25) (54,57). Dataset-wide unsupervised clustering returned 519 differentially expressed gene signatures of upregulated genes.

For unsupervised clustering on the sample level, cancer epithelial cells were clustered by sample at 15 resolutions (0.01, 0.05, 0.08, 0.1, 0.2, 0.3, 0.4, 0.5, 0.6, 0.7, 1.0, 1.3, 1.6, 1.8, 2.0) utilizing Seurat (v4.1.0) (54). FindMarkers in Seurat (v4.1.0) and MAST (v1.20.0) were then used to identify differentially expressed genes in each cluster (>5 cells per cluster, only test genes with >25% difference in the fraction of detection between the clusters, log₂FC>0.25) (54,57). Sample-level unsupervised clustering returned 5,546 differentially expressed gene signatures of upregulated genes.

For supervised clustering by SC50 molecular subtype (i.e., basal, HER2-enriched, luminal A, luminal B), epithelial cells were clustered based on SC50 subtype (8). FindMarkers in Seurat (v4.1.0) and MAST (v1.20.0) were then used to identify differentially expressed genes in each cluster (>5 cells per cluster, only test genes detected in >20% of cells in a cluster, log₂FC>0.1) (54,57). Supervised clustering based on clinical target expression returned 4 differentially expressed gene signatures of upregulated genes.

For supervised clustering based on clinical target expression (ESR1, ERBB2, ERBB3, PIK3CA, NTRK1/NTRK2/NTRK3, CD274, EGFR, FGFR1/FGFR2/FGFR3/FGFR4, TACSTD2, CDK4/CDK6, AR, NECTIN2), epithelial cells were scored using UCell (v1.99.1) and clustered based on high (expression level above the top 90th percentile of all epithelial cells), medium (expression level below the top 90th percentile but greater than zero), and low (no or zero expression level) expression of clinical targets (56). FindMarkers in Seurat (v4.1.0) and MAST (v1.20.0) were then used to identify differentially expressed genes in each cluster (>5 cells per cluster, only test genes detected in >20% of cells in a cluster, log₂FC>0.1) (54,57). Supervised clustering based on clinical target expression returned 32 differentially expressed gene signatures of upregulated genes.

Only gene signatures containing more than 20 genes and originating from clusters of >5 epithelial cells were kept. Additionally, redundancy was reduced by comparing all pairs of unsupervised gene signatures and removing the pair with the fewest genes from pairs with Jaccard index>0.9. Across all tumor samples, a total of 1,101 gene signatures were generated.

Consensus clustering of the Jaccard similarities (using skmeans clustering, ATC, implemented in the cola (v2.0.0) package) between these gene signatures was used to identify 10 groupings (FIG. 12A-12B) (64). For each grouping, we took the top 100 genes that have the highest frequency of occurrence across clusters. These were defined as a gene element (i.e., GE) and were named GE1 to GE10. GE signature expression was calculated for each cancer epithelial cell using UCell (v1.99.1) (56). For FIGS. 3B and 3C, GE signature expression was z-scored across all cancer epithelial cells, and each cell was assigned to the GE with the highest z-scored expression.

### Survival Analysis

To determine survival outcomes, we obtained the primary solid tumor samples from the breast cancer cohort of The Cancer Genome Atlas (TCGA) Project (66). Genes names were converted using the same method described in the scRNA-seq processing section. The data was normalized using the TCGAAnalyze_Normalization function from the TCGAbiolinks (v2.18.0) package with default parameters and was subsequently transformed using the vst function from the DESeq2 (v1.34.0) package, also with default parameters (67,68).

We assessed survival outcomes on the TCGA samples with high immune infiltrate (activated and resting NK cells predicted to be greater than a relative fraction of 0.015 of tumor-infiltrating immune cells in the sample), as defined by Xu et al. (9). For these samples, we applied NK-specific genes (NCAM1, FGFBP2, KLRD1, FCGR3A, KLRK1) and the 44 upregulated genes of the INK signature to all TCGA samples and labeled the top 300 patients with highest rNK signature expression as ‘rNK-high’ and the bottom 801 patients with lowest rNK signature expression as ‘rNK-low.” Survival curves were generated using the Kaplan-Meier method with the survival package (v2.44-1.1) (69). We assessed the significance between two groups using log-rank test statistics. Patients >45 yo demonstrated worse outcomes with increased INK cell signature expression (p<0.05) (FIG. 9F); survival analysis for patients <45 yo did not show significance, though there was a similar trend. To ensure age was not a confounder, correlation between age at initial diagnosis and survival was also assessed (R=−0.11, p>0.05) (FIG. 9E).

### Receptor-Ligand Pairing Analysis Between Cancer Epithelial Cells and Immune Cells

To identify interactions that may influence NK cell reprogramming, separate NicheNet analyses were run between rNK cells and cancer epithelial cells separated by clinical subtype (HER2+, HR+, TNBC), where rNK cells were set as the ‘sender’ population, and non-rNK were set as the ‘reference’) (70). Receptor-ligand regulatory potential scores for the top 50 predicted ligands and top 200 predicted targets were calculated and for each predicted receptor-ligand pair, an R-L interaction score was calculated as a product of ligand expression (fold change in average expression of the ligand in cancer epithelial cells of that clinical subtype) and receptor expression (percent of the rNK population that has positive expression of the receptor). For the top 20 receptor-ligand pairs selected based on this R-L interaction score, circos plots were generated to visualize links between ligands on the cancer epithelial cells by GE and receptors on the interacting cell subsets.

Next, receptor-ligand pairing analysis was performed using NicheNet (v1.1.0) and CellChat (v0.0.1) to explore interactions between cancer epithelial cells and interacting immune and stromal cells (i.e., CD4+ T cells, CD8+ T cells, regulatory T cells, B cells, plasma cells, myeloid cells, mast cells, MDSCs, NK cells, rNK cells, fibroblasts, myoepithelial cells, endothelial cells, perivascular-like cells) (70,71). For each cancer epithelial cell, GE signature expression was z-scored across all cancer epithelial cells, and each cell was assigned to the GE with the highest z-scored expression. For each GE, in a similar manner as with the rNK analyses, multiple separate NicheNet analyses were run between cancer epithelial cells assigned to that GE (set as ‘sender’ population) and each interacting cell subset (set as ‘receiver’ population). The top 50 predicted ligands and top 200 predicted targets were used for the development of the R-L interaction score, which was the product of the fold change in average expression of the ligand on cancer epithelial cells with high vs. low GE expression, and receptor expression (percent of the interacting cell subset that has positive expression of the receptor). For the top 20 receptor-ligand pairs selected based on this R-L interaction score, circos plots were again generated to visualize links between ligands on the cancer epithelial cells by GE and receptors on the interacting cell subsets.

In addition to the NicheNet analysis, cancer cell and interacting cell communication analysis was conducted using CellChat (v0.0.1) with default parameters (71). All cancer epithelial cells were assigned to the GE with the highest z-scored expression. For each GE, the cell-cell communication network between GE-labeled cancer epithelial cells and interacting cells was visualized using CellChat (v0.0.1) (104). For each GE and interacting cell pair, receptor-ligand pairings with significant (Bonferroni adjusted p-value<0.05) probability of interaction are selected as a curated list.

To infer the degree of interaction between the GEs and various immune or stromal cell populations, we estimate the number of key receptor-ligand interactions for each GE and interacting cell population. First, the list of receptor-ligand interactions predicted by NicheNet were filtered. For each interacting cell population, the top 2,000 predicted receptor-ligands for each interacting cell population were selected based on Nichenet prediction for regulatory potential. Then, of those selected pairs, the top 400 predicted receptor-ligands for each GE were selected based on ligand expression (fold change in average expression of the ligand on cancer epithelial cells with high vs. low GE expression). Lastly, all overlapping receptor-ligand interactions that were predicted by both NicheNet and CellChat for a GE and interacting cell pair are selected. We combined the list of overlapping receptor-ligand interactions and the list of selected NicheNet receptor-ligand interactions to generate our list of curated receptor-ligand interactions for each GE and immune or stromal cell population.

For each GE and interacting cell pair, the number of curated receptor-ligand interactions was used to infer the degree of interaction between the GE and the interacting cell population. We visualized the scaled number of curated receptor-ligand interactions in our GE-immune interaction decoder matrix (FIG. 3E). We also visualized the absolute number of curated receptor-ligands between each GE and interacting cell (FIG. 12D)

### Breast Cancer Cell Line Exploration

To explore cancer epithelial cell heterogeneity and NK cell sensitivity, we obtained bulk RNA-seq data for primary breast cancer cell lines from the Broad Cancer Cell Line Encyclopedia (CCLE) differentiated by their sensitivity to NK cells (34,35). Briefly, data from CCLE contained TPM values of protein coding genes were inferred from RNA-seq data using the RSEM tool and were reported after log 2 transformation, using a pseudo-count of 1; log 2(TPM+1). GE signature expression was calculated for each breast cancer cell line using UCell (v1.99.1).

We experimentally confirmed NK cell cytotoxicity against select human breast cancer cell lines. We selected the HCC1954 cell line which had increased expression of NK-resistant GEs (GE1 and GE6) and the MCF7 cell line which had decreased expression of NK-resistance GEs. Additionally, the K562 cell line (derived from human myelogenous leukemia) is known to be sensitive to NK cell killing and therefore served as a positive control (49, 92, 93). The NK-92 cell line, a highly cytotoxic and IL-2-dependent NK cell line derived from a patient with non-Hodgkin's lymphoma, was cultured in media with IL-2. To determine killing function of NK cells against the cell lines, HCC1954, MCF7, and K562 cells were cocultured with NK-92 at a ratio of 1:5 in 48-well plates for 12 hours at 37° C., and then the supernatant was collected for lactate dehydrogenase (LDH) assay following the manufacturer's instructions (CyQuant LDH cytotoxicity assay kit, Invitrogen). Absorbance was measured at 490 nm and 600 nm (background) via microplate. The killing function of NK cells was reported as percentage LDH produced (% LDH production=(sample absorbance−spontaneous absorbance)/(maximum absorbance−spontaneous absorbance)). Higher NK cytotoxicity was inferred based on increased LDH production.

From Sheffer et al. (49), breast cancer cell line sensitivity to NK cell killing was assessed using reported 72-hour AUC values. Briefly, Sheffer et al. performed a PRISM-based phenotypic screen with pools of DNA-barcoded cell lines to quantify NK cell cytotoxicity against cancer cell lines using the AUC of tumor cell survival. Please refer to the original study (49) for additional information. For breast cancer cell lines, NK cell sensitivity was based on the reported 72-hour AUC values from the Sheffer et al. study. Linear regression and Pearson correlation were used to assess the relationship between GE expression and NK cell sensitivity for breast cancer cell lines.

### Spatial Transcriptomics Analysis

Processed spatial transcriptomics count matrices for 6 samples from Wu et al. were loaded into Seurat (v.4.1.0) (8,54). Because each spot captures multiple cells, we deconvoluted the underlying composition of cell types using the anchor-based Seurat integration workflow (FIG. 13A). This workflow used SCTransform normalization (55) and transferred annotations from the integrated scRNA-seq dataset reference to the spatial transcriptomic datasets. The resulting annotations calculated the fraction of each cell type per given spot and mapped the spatial distribution of cell types, which we further corroborated by the spatial expression of marker genes (Table 3). Spatial and scRNA-seq data were matched with respect to breast cancer clinical subtype. Spots labeled as normal tissue or artefact by pathologist annotation were excluded from the analysis.

To investigate interactions between cancer epithelial cells by GE and immune or stromal cells, spots were first filtered based on the predicted scores of the ‘cancer epithelial cell’ annotation called by the Seurat integration (spots with less than 10% predicted cancer epithelial cells are excluded). Each spot containing cancer epithelial cells was then scored for expression of each of the 10 GEs using UCell (v1.99.1. For immune and stromal cell populations, spots were filtered based on predicted scores for their respective annotations called by the Seurat integration (spots with 0% predicted cells are excluded). Each spot containing the respective cell type was scored for expression of that cell using canonical and literature-derived cell markers (Table 3). For each immune and stromal cell population, each spot containing the respective cell type was scored for expression of the cell using canonical and literature-derived cell markers by the UCell (v1.99.1) package. To assess GE and CD8+ T cell colocalization for all samples, Pearson correlations were computed across spots containing between the expression of each GE and the expression of CD8+ T cell markers. For cell signaling predictions between select GE ligands and CD8+ T cell receptors, receptor-ligand co-localization scores were defined as the product of the ligand and receptor normalized expression levels.

### Development of InteractPrint, a Cancer Epithelial Cell-Immune Cell Interaction Score

For each sample, the average expression of each GE is calculated as the average of the scaled UCell score (scaled across all cancer epithelial cells in the dataset). Next, the number of prioritized receptor-ligand interactions in the GE-immune reference matrix between each GE and CD8+ T cells is used to infer the degree of interaction between cancer epithelial cells and CD8+ T cells. GE1, GE6, GE7, GE8, and GE9 were designated as “inactivating” based on the presence inactivating CD8+ T cell receptors (e.g., NECTIN2_TIGIT) in the list of prioritized receptor-ligand interactions for those GEs.

For each sample, the T cell InteractPrint was calculated as the average of the number of curated CD8+ T cell receptor-ligand interactions in the GE-immune interaction reference matrix, weighted by average expression of each GE and a factor of −1 for inactivating GEs.

$$IP = {\sum }_{i = 1}^{10} ( {e}_{i} ) ( {R}_{i} ) ( w )$$

Weighted CD8+ T cell interaction score calculated for a patient tumor

i=GE (ranges from 1 to 10),

eᵢ=Average GE expression (average of z scored UCell scores for the GE across all cells in the sample),

Rᵢ=Number of curated R-L pairs (from GE-immune interaction decoder matrix).

w=Multiplier for activating or inactivating GE (w=1 for CD8+ T cell activating GEs; w=−1 for CD8+ T cell inactivating GEs)

### Validation of InteractPrint

To assess the predictive value of the T cell InteractPrint, we applied our method to a publicly available scRNA-seq dataset containing 29 primary breast tumors from patients who received pembrolizumab anti-PD-1 therapy (Bassez et al.) (36). In Bassez et al, response was inferred based on T cell clonal expansion, as determined by sTCR-seq of pre- and on-treatment samples (53). To determine cancer epithelial cells in the Bassez et al. dataset, CNV analysis was performed (FIG. 7A-7D). GE signature expression was calculated for each pre-treatment sample using UCell (v1.99.1) (56), and the T cell InteractPrint was calculated for each sample.

To further assess the predictive value of InteractPrint, we applied our method to the I-SPY 2 microarray dataset containing 69 primary breast tumors from patients who received combination paclitaxel and pembrolizumab anti-PD-1 therapy. The data was loaded using limma (v3.15), and the batch-corrected and normalized expression data provided by the authors was inserted into the object. Genes names were converted using the same method described in the scRNA-seq processing section. Microarray data was deconvoluted with BisqueRNA (v1.0.5) using marker-based devolution with the 10 GE signatures in order to estimate the relative abundance of the GEs within each sample. GE signature expression was compared for responders and non-responders.

On both datasets, we assessed the predictive value of the T cell InteractPrint compared to average expression levels of PD-L1. ROC curves and AUC statistics were generated using the PROC (v1.18.0) and default settings. Bootstrap method (n=10,000) in PROC (v1.18.0) was used for significance testing between T cell InteractPrint ROC and PD-L1 ROC curves.

### Statistical Analyses

Statistical significance was determined using the Wilcoxon Rank Sum test (unless otherwise stated in the figure legend). Where appropriate, p-values were adjusted using the Bonferroni correction (unless otherwise stated in the figure legend) where appropriate for multiple testing. All box plots depict the first and third quartiles as the lower and upper bounds, respectively. The whiskers represent 1.5× the interquartile range and the center depicts the median. All statistical tests used are defined in the figure legends. P-values<0.05 were considered significant.

### Data and Code Availability

Upon publication, all processed scRNA-seq data in the integrated dataset will be made available for in-browser exploration and visualization through the Broad Institute Single Cell portal at https://singlecell.broadinstitute.org/single_cell/. Processed scRNA-seq data from this study will also be made available for download through the Gene Expression Omnibus upon publication.

Six of the nine publicly available scRNA-seq datasets were retrieved from the Gene Expression Omnibus under the following accession codes: GSE114727 (Azizi et al. (7)), GSE118389 (Karaayvaz et. al., (8)), GSE161529 (Pal et al. (9)), GSE110686 (Savas et al. (10)), GSE176078 (Wu et al. (13)), and GSE180286 (Xu et al. (14)). The remaining three scRNA-seq datasets can be found at the following links: lambrechtslab.sites.vib.be/en/single-cell (Bassez et al. (53)), lambrechtslab.sites.vib.be/en/pan-cancer-blueprint-tumour-microenvironment-0 et (Qian al. (11)), and singlecell.broadinstitute.org/single_cell/study/SCP1106/stromal-cell-diversity-associated-with-immune-evasion-in-human-triple-negative-breast-cancer (Wu et al. (13)). The spatially resolved data from Wu et al. were retrieved from the Zenodo data repository (https://doi.org/10.5281/zenodo.4739739). The breast cancer TCGA expression data was downloaded using the TCGAbiolinks package. Breast cancer cell lines were downloaded from the depmap portal at the following link: https://depmap.org/portal/download/. The I-SPY2-990 microarray data was retrieved from the Gene Expression Omnibus under the accession code GSE194040.

Code related to these analyses will be made available at github.com/ChanLab-UTSW/BreastCancer_Integrated upon publication. Additional data and methods are available from the corresponding author upon request.

### Example 11—rNK Cell Signature Identified in Pancreatic Tumor Microenvironment

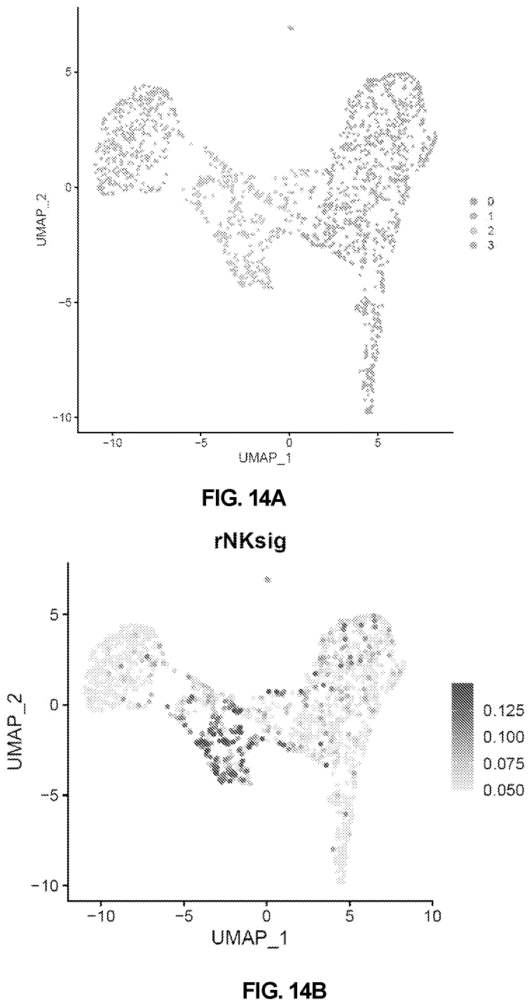

The examples above all center on the breast tumor microenvironment. To determine whether the gene expression signatures identified above are applicable in other microenvironments, additional experiments were conducted in human pancreatic tumors. Single tumor epithelial cells in the tumors were clustered in an unbiased manner as shown in FIG. 14A using methods described in previous examples. The same cells were then analyzed using the reprogrammed NK (rNK) cell signature described above to determine whether any natural cluster correlated with high expression of the rNK cell signature. Indeed, cluster 2 was shown to correlate strongly with the INK cell signature (FIG. 14B) suggesting that reprogrammed NK cells are a natural phenomenon in multiple tumor microenvironments.

### Example 12—Gene Expression Profiling Predicts Cellular Sensitivity to Immunotherapy

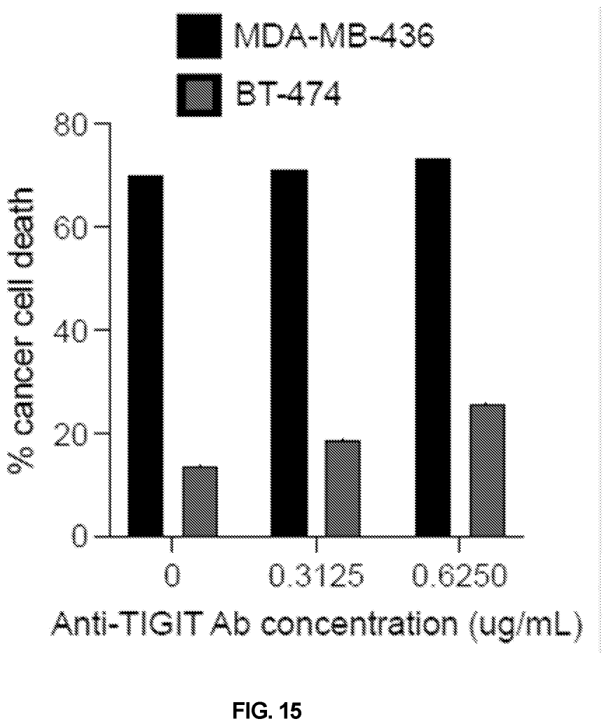

Breast cancer cell lines (MDM-MB-436 and BT-474) were classified into GE classes based on their expression of GE genes. MDM-MB-436 cells were classified as GE5 cells (high in GE5 gene expression), and BT-474 were classified as GE1/6 cells (high in GE1 and GE6 gene expression). It was predicted that GE 5 cells would be very sensitive to NK Cell killing (MDA-MB-436) and cells that were GE1/6 high (BT-474) to be not sensitive to NK cell killing. This was shown when each cell population was co-cultured with NK cells (NK-92) (see FIG. 15, left columns). However, it was also predicted that BT-474 would be most sensitive to a specific immune checkpoint inhibitor (e.g., an anti-TIGIT block therapy) based on predicted receptor/ligand pairing for GE1 and GE6, which included TIGIT (T-cell immunoglobulin and ITIM domain) receptors. Indeed when cultures of cancer cells (either MDA-MB-436 or BT-474) and NK cells (NK-92) were treated with anti-TIGIT blocking antibodies (0.3125 μg/mL or 0.625 μg/mL), only BT-474 cells showed a detectable effect—with killing increased by 2-fold (see FIG. 15, middle and right columns). This suggests that GE expression profiles described above can directly predict responsiveness to an immunotherapy.

### REFERENCES

1. Siegel R L, Miller K D, Fuchs H E, and Jemal A. Cancer statistics, 2022. CA: A Cancer Journal for Clinicians. 2022; 72 (1): 7-33.
2. Hanker A B, Sudhan D R, and Arteaga C L. Overcoming Endocrine Resistance in Breast Cancer. Cancer Cell. 2020; 37 (4): 496-513.
3. Al-Hajj M, Wicha M S, Benito-Hernandez A, Morrison S J, and Clarke M F. Prospective identification of tumorigenic breast cancer cells. Proc Natl Acad Sci USA. 2003; 100 (7): 3983-8.
4. Feng Y, Spezia M, Huang S, Yuan C, Zeng Z, Zhang L, et al. Breast cancer development and progression: Risk factors, cancer stem cells, signaling pathways, genomics, and molecular pathogenesis. Genes Dis. 2018; 5 (2): 77-106.
5. Place A E, Jin Huh S, and Polyak K. The microenvironment in breast cancer progression: biology and implications for treatment. Breast Cancer Research. 2011; 13 (6): 227.
6. Polyak K. Breast cancer: origins and evolution. The Journal of Clinical Investigation. 2007; 117 (11): 3155-63.
7. Azizi E, Carr A J, Plitas G, Cornish A E, Konopacki C, Prabhakaran S, et al. Single-Cell Map of Diverse Immune Phenotypes in the Breast Tumor Microenvironment. Cell. 2018; 174 (5): 1293-308 e36.
8. Karaayvaz M, Cristea S, Gillespie S M, Patel A P, Mylvaganam R, Luo C C, et al. Unravelling subclonal heterogeneity and aggressive disease states in TNBC through single-cell RNA-seq. Nat Commun. 2018; 9.
9. Pal B, Chen Y S, Vaillant F, Capaldo B D, Joyce R, Song X Y, et al. A single-cell RNA expression atlas of normal, preneoplastic and tumorigenic states in the human breast. Embo Journal. 2021; 40 (11).
10. Savas P, and Loi S. Expanding the Role for Immunotherapy in Triple-Negative Breast Cancer. Cancer Cell. 2020; 37 (5): 623-4.
11. Qian J B, Olbrecht S, Boeckx B, Vos H, Laoui D, Etlioglu E, et al. A pan-cancer blueprint of the heterogeneous tumor microenvironment revealed by single-cell profiling. Cell Res. 2020; 30 (9): 745-62.
12. Wu S Z, Roden D L, Wang C, Holliday H, Harvey K, Cazet A S, et al. Stromal cell diversity associated with immune evasion in human triple-negative breast cancer. The EMBO Journal. 2020; 39 (19).
13. Wu S Z, Al-Eryani G, Roden D L, Junankar S, Harvey K, Andersson A, et al. A single-cell and spatially resolved atlas of human breast cancers. Nature Genetics. 2021; 53 (9): 1334-47.
14. Xu K, Wang R, Xie H, Hu L, Wang C, Xu J, et al. Single-cell RNA sequencing reveals cell heterogeneity and transcriptome profile of breast cancer lymph node metastasis. Oncogenesis. 2021; 10 (10).
15. Chan I S, KnUtsd0ttir H, Ramakrishnan G, Padmanaban V, Warrier M, Ramirez J C, et al. Cancer cells educate natural killer cells to a metastasis-promoting cell state. Journal of Cell Biology. 2020; 219 (9).
16. Chan I S, and Ewald A J. The changing role of natural killer cells in cancer metastasis. Journal of Clinical Investigation. 2022; 132 (6).
17. Melaiu O, Lucarini V, Cifaldi L, and Fruci D. Influence of the Tumor Microenvironment on NK Cell Function in Solid Tumors. Frontiers in Immunology. 2020; 10.
18. Polyak K. Heterogeneity in breast cancer. J Clin Invest. 2011; 121 (10): 3786-8.
19. Turashvili G, and Brogi E. Tumor Heterogeneity in Breast Cancer. Front Med (Lausanne). 2017; 4:227.
20. Crinier A, Dumas P-Y, Escaliere B, Piperoglou C, Gil L, Villacreces A, et al. Single-cell profiling reveals the trajectories of natural killer cell differentiation in bone marrow and a stress signature induced by acute myeloid leukemia. Cellular & Molecular Immunology. 2021; 18 (5): 1290-304.
21. Smith S L, Kennedy P R, Stacey K B, Worboys J D, Yarwood A, Seo S, et al. Diversity of peripheral blood human NK cells identified by single-cell RNA sequencing. Blood Advances. 2020; 4 (7): 1388-406.
22. Yang C, Siebert J R, Burns R, Gerbec Z J, Bonacci B, Rymaszewski A, et al. Heterogeneity of human bone marrow and blood natural killer cells defined by single-cell transcriptome. Nature Communications. 2019; 10 (1): 3931.
23. de Andrade L F, Lu Y, Luoma A, Ito Y, Pan D, Pyrdol J W, et al. Discovery of specialized NK cell populations infiltrating human melanoma metastases. JCI Insight. 2019; 4 (23).
24. Moreno-Nieves U Y, Tay J K, Saumyaa S, Horowitz N B, Shin J H, Mohammad I A, et al. Landscape of innate lymphoid cells in human head and neck cancer reveals divergent NK cell states in the tumor microenvironment. Proceedings of the National Academy of Sciences. 2021; 118 (28): e2101169118.
25. Cheon H, Borden E C, and Stark G R. Interferons and their stimulated genes in the tumor microenvironment. Semin Oncol. 2014; 41 (2): 156-73.
26. Dogra P, Rancan C, Ma W, Toth M, Senda T, Carpenter D J, et al. Tissue Determinants of Human NK Cell Development, Function, and Residence. Cell. 2020; 180 (4): 74963.e13.
27. Chen J. L0pez-Moyado I F, Seo H, Lio C-W J, Hempleman L J, Sekiya T, et al. NR4A transcription factors limit CAR T cell function in solid tumours. Nature. 2019; 567 (7749): 530-4.
28. Zhou F. Drabsch Y, Dekker T J A, De Vinuesa A G, Li Y, Hawinkels L J A C, et al. Nuclear receptor NR4A1 promotes breast cancer invasion and metastasis by activating TGF-r3 signalling. Nat Commun. 2014; 5 (1).
29. Chan C J, Martinet L, Gilfillan S, Souza-Fonseca-Guimaraes F, Chow M T, Town L, et al. The receptors CD96 and CD226 oppose each other in the regulation of natural killer cell functions. Nature Immunology. 2014; 15 (5): 431-8.
30. Braud V M, Allan D S J, O'Callaghan C A, S6derstr6m K, D'Andrea A, Ogg G S, et al. HLA-E binds to natural killer cell receptors CD94/NKG2A, B and C. Nature. 1998; 391 (6669): 795-9.
31. Xu K, Wang R, Xie H, Hu L, Wang C, Xu J, et al. Single-cell RNA sequencing reveals cell heterogeneity and transcriptome profile of breast cancer lymph node metastasis. Oncogenesis. 2021; 10 (10): 66.
32. Modi S, Jacot W, Yamashita T, Sohn J, Vidal M, Tokunaga E, et al. Trastuzumab Deruxtecan in Previously Treated HER2-Low Advanced Breast Cancer. New England Journal of Medicine. 2022.
33. Rugo H S, Bardia A, Marme F, Cortes J, Schmid P, Loirat D, et al. Primary results from TROPICS-02: A randomized phase 3 study of sacituzumab govitecan (SG) versus treatment of physician's choice (TPC) in patients (Pts) with hormone receptor—positive/HER2-negative (HR+/HER2−) advanced breast cancer. Journal of Clinical Oncology. 2022; 40 (17_suppl): LBA 1001-LBA.
34. Li X, and Wang C-Y. From bulk, single-cell to spatial RNA sequencing. International Journal of Oral Science. 2021; 13 (1): 36.
35. Tan R S Y C, Ong W S, Lee K-H, Lim A H, Park S, Park Y H, et al. HER2 expression, copy number variation and survival outcomes in HER2-low non-metastatic breast cancer: an international multicentre cohort study and TCGA-METABRIC analysis. BMC Medicine. 2022; 20 (1).
36. Schettini F, Chic N, Bras0-Maristany F, Pare L, Pascual T, Conte B, et al. Clinical, pathological, and PAM50 gene expression features of HER2-low breast cancer. npj Breast Cancer. 2021; 7 (1).
37. Vidula N, Yau C, and Rugo H. Trophoblast Cell Surface Antigen 2 gene (TACSTD2) expression in primary breast cancer. Breast Cancer Research and Treatment. 2022.
38. Ambrogi F, Fornili M, Boracchi P, Trerotola M, Relli V, Simeone P, et al. Trop-2 Is a Determinant of Breast Cancer Survival. PLoS ONE. 2014; 9 (5): e96993.
39. Aslan M, Hsu E-C, Garcia-Marques F J, Bermudez A, Liu S, Shen M, et al. Oncogene-mediated metabolic gene signature predicts breast cancer outcome. npj Breast Cancer. 2021; 7 (1): 141.
40. Rizeq B, Zakaria Z, and Ouhtit A. Towards understanding the mechanisms of actions of carcinoembryonic antigen-related cell adhesion molecule 6 in cancer progression. Cancer ScL 2018; 109 (1): 33-42.
41. Kanda Y, Mizuno A, Takasaki T, Satoh R, Hagihara K, Masuko T, et al. Down-regulation of dual-specificity phosphatase 6, a negative regulator of oncogenic ERK signaling, by ACA-28 induces apoptosis in NIH/3T3 cells overexpressing HER2/ErbB2. Genes Cells. 2021; 26 (2): 109-16.
42. Desai K, Nair M G, Prabhu J S, Vinod A, Korlimarla A, Rajarajan S, et al. High expression of integrin p6 in association with the Rho-Rac pathway identifies a poor prognostic subgroup within HER2 amplified breast cancers. Cancer Med. 2016; 5 (8): 2000-11.
43. Cheung K J, Gabrielson E, Werb Z, and Ewald A J. Collective invasion in breast cancer requires a conserved basal epithelial program. Cell. 2013; 155 (7): 1639-51.
44. Seol H, Lee H J, Choi Y, Lee H E, Kim Y J, Kim J H, et al. Intratumoral heterogeneity of HER2 gene amplification in breast cancer: its clinicopathological significance. Modern Pathology. 2012: 25 (7): 938-48.
45. Janiszewska M, Liu L, Almendro V, Kuang Y, Paweletz C, Sakr R A, et al. In situ single-cell analysis identifies heterogeneity for PIK3CA mutation and HER2 amplification in HER2-positive breast cancer. Nature Genetics. 2015; 47 (10): 1212-9.
46. Soucheray M, Capelletti M, Pulido I, Kuang Y, Paweletz C P, Becker J H, et al. Intratumoral Heterogeneity in <i>EGFR</i>-Mutant NSCLC Results in Divergent Resistance Mechanisms in Response to EGFR Tyrosine Kinase Inhibition. Cancer Research. 2015; 75 (20): 4372-83.
47. Muzumdar M D, Dorans K J, Chung K M, Robbins R, Tammela T, Gocheva V, et al. Clonal dynamics following p53 loss of heterozygosity in Kras-driven cancers. Nature Communications. 2016; 7 (1): 12685.
48. Liu B, Li C, Li Z, Wang D, Ren X, and Zhang Z. An entropy-based metric for assessing the purity of single cell populations. Nat Commun. 2020; 11 (1): 3155.
49. Sheffer M, Lowry E, Beelen N, Borah M, Amara S N-A, Mader C C, et al. Genome-scale screens identify factors regulating tumor cell responses to natural killer cells. Nature Genetics. 2021; 53 (8): 1196-206.
50. Barretina J, Caponigro G, Stransky N, Venkatesan K, Margolin A A, Kim S, et al. The Cancer Cell Line Encyclopedia enables predictive modelling of anticancer drug sensitivity. Nature. 2012; 483 (7391): 603-7.
51. Network NCC. Breast Cancer (v4.2022). //www.nccn.org/professionals/physician_gls/pdf/breast.pdf Accessed Oct. 1, 2022.
52. Pardoll D M. The blockade of immune checkpoints in cancer immunotherapy. Nat Rev Cancer. 2012; 12 (4): 252-64.
53. Bassez A, Vos H, Van Dyck L, Floris G, Arijs I, Desmedt C, et al. A single-cell map of intratumoral changes during anti-PD1 treatment of patients with breast cancer. Nature Medicine. 2021; 27 (5): 820-32.
54. Nanda R. Liu M C, Yau C, Shatsky R, Pusztai L, Wallace A, et al. Effect of Pembrolizumab Plus Neoadjuvant Chemotherapy on Pathologic Complete Response in Women With Early-Stage Breast Cancer: An Analysis of the Ongoing Phase 2 Adaptively Randomized I-SPY2 Trial. JAMA Oncology. 2020; 6 (5): 676-84.
55. Wu N C, Wong W, Ho K E, Chu V C, Rizo A, Davenport S, et al. Comparison of central laboratory assessments of ER, PR, HER2, and Ki67 by IHC/FISH and the corresponding mRNAs (ESR1, PGR, ERBB2, and MKi67) by RT-qPCR on an automated, broadly deployed diagnostic platform. Breast Cancer Res Treat. 2018; 172 (2): 327-38.
56. Denkert C, Huober J, Loibl S, Prinzler J, Kronenwett R, Darb-Esfahani S, et al. HER2 and ESR1 mRNA expression levels and response to neoadjuvant trastuzumab plus chemotherapy in patients with primary breast cancer. Breast Cancer Res. 2013; 15 (1): R11.
57. Wang Z, Portier B P, Gruver A M, Bui S, Wang H, Su N, et al. Automated quantitative RNA in situ hybridization for resolution of equivocal and heterogeneous ERBB2 (HER2) status in invasive breast carcinoma. J Mol Diagn. 2013; 15 (2): 210-9.
58. Vassilakopoulou M, Togun T, Dafni U, Cheng H, Bordeaux J, Neumeister V M, et al. In situ quantitative measurement of HER2mRNA predicts benefit from trastuzumab-containing chemotherapy in a cohort of metastatic breast cancer patients. PLoS One. 2014; 9 (6): e99131.
59. Coates J T, Sun S, Leshchiner I, Thimmiah N, Martin E E, McLoughlin D, et al. Parallel Genomic Alterations of Antigen and Payload Targets Mediate Polyclonal Acquired Clinical Resistance to Sacituzumab Govitecan in Triple-Negative Breast Cancer. Cancer Discov. 2021; 11 (10): 2436-45.
60. Zhao W, Kuai X, Zhou X, Jia L, Wang J, Yang X, et al. Trop2 is a potential biomarker for the promotion of EMT in human breast cancer. Oncol Rep. 2018; 40 (2): 759-66.
61. Chou J. Trepka K, Sjostrom M, Egusa E A, Chu C E, Zhu J, et al. TROP2 Expression Across Molecular Subtypes of Urothelial Carcinoma and Enfortumab Vedotin-resistant Cells. Eur Urol Oncol. 2022; 5 (6): 714-8.
62. Ohmachi T, Tanaka F, Mimori K, Inoue H, Yanaga K, and Mori M. Clinical significance of TROP2 expression in colorectal cancer. Clin Cancer Res. 2006; 12 (10): 3057-63.
63. Bignotti E, Todeschini P, Calza S, Falchetti M, Ravanini M, Tassi R A, et al. Trop-2 overexpression as an independent marker for poor overall survival in ovarian carcinoma patients. Eur J Cancer. 2010; 46 (5): 944-53.
64. Stepan L P, Trueblood E S, Hale K, Babcook J, Borges L, and Sutherland C L. Expression of Trop2 cell surface glycoprotein in normal and tumor tissues: potential implications as a cancer therapeutic target. J Histochem Cytochem. 2011; 59 (7): 701-10.
65. Rugo H S, Delord J P, Im S A, Ott P A, Piha-Paul S A, Bedard P L, et al. Safety and Antitumor Activity of Pembrolizumab in Patients with Estrogen Receptor-Positive/Human Epidermal Growth Factor Receptor 2-Negative Advanced Breast Cancer. Clin Cancer Res. 2018; 24 (12): 2804-11.
66. Kwa M J, and Adams S. Checkpoint inhibitors in triple-negative breast cancer (TNBC): Where to go from here. Cancer. 2018; 124 (10): 2086-103.
67. Gandhi L, Rodriguez-Abreu D, Gadgeel S, Esteban E, Felip E, De Angelis F, et al. Pembrolizumab plus Chemotherapy in Metastatic Non-Small-Cell Lung Cancer. N Engl J Med. 2018; 378 (22): 2078-92.
68. Garon E B, Rizvi N A, Hui R, Leigh! N, Balmanoukian A S, Eder J P, et al. Pembrolizumab for the treatment of non-small-cell lung cancer. N Engl J Med. 2015; 372 (21): 2018-28.
69. Schachter J, Ribas A, Long G V, Arance A, Grob J J, Mortier L, et al. Pembrolizumab versus ipilimumab for advanced melanoma: final overall survival results of a multicentre, randomised, open-label phase 3 study (KEYNOTE-006). Lancet. 2017; 390 (10105): 185362.
70. Bellmunt J, de Wit R, Vaughn D J, Fradet Y, Lee J L, Fong L, et al. Pembrolizumab as Second-Line Therapy for Advanced Urothelial Carcinoma. N Engl J Med. 2017; 376 (11): 1015-26.
71. Ritchie M E, Phipson B, Wu D, Hu Y, Law C W, Shi W, et al. limma powers differential expression analyses for RNA-sequencing and microarray studies. Nucleic Acids Research. 2015; 43 (7): e47-e.
72. Carlson M. org.Hs.eg.db: Genome wide annotation for Human. R package version 3.8.2, 2019.
73. McGinnis C S, Murrow L M, and Gartner Z J. DoubletFinder: Doublet Detection in Single-Cell RNA Sequencing Data Using Artificial Nearest Neighbors. Cell Systems. 2019; 8 (4): 329-37.e4.
74. Hao Y, Hao S, Andersen-Nissen E, Mauck W M, Zheng S, Butler A, et al. Integrated analysis of multimodal single-cell data. Cell. 2021; 184 (13): 3573-87.e29.
75. Hafemeister C, and Satija R. Normalization and variance stabilization of single-cell RNA-seq data using regularized negative binomial regression. Genome Biology. 2019; 20 (1): 296.

1. A method of classifying a cancer epithelial cell to a gene element group, the method comprising:
(i) obtaining a set of expressed genes in the cancer epithelial cell;
(ii) determining expression levels of genes in a plurality of gene sets and ranking the expression levels in each gene set to identify a gene set having highest gene expression; and
(iii) assigning the cell to a gene element group corresponding to the gene set having highest gene expression.

2. The method of claim 1, wherein the cancer epithelial cell is assigned to:
(a) a gene element 1 group (GE1) when the gene set having highest gene expression comprises at least one gene, at least 5 genes, at least 10 genes, at least 15 genes, or at least 20 genes selected from the group consisting of AC090498-1, AC105999-2, ADIRF, AGR2, AGR3, ALDH2, ANKRD30A, ARL6IP1, ARMT1, ATAD2, AZGP1, BATF, BMPR1B, BST2, BTG2, C15ORF48, CCDC74A, CEBPD, CFD, CLDN4, CLU, COX6C, CPB1, CRIP1, CST3, CTHRC1, CXCL14, DHRS2, DSCAM-AS1, ELF3, ELP2, ERBB4, ESR1, EVL, FABP3, FHL2, FKBP5, FSIP1, GJA1, GSTM3, HES1, HSPB1, IFI27, IF16, IFITM1, IFITM2, IFITM3, IGFBP4, INPP4B, ISG15, JUNB, KCNE4, KCNJ3, KRT18, KRT19, LDLRAD4, MAGED2, MDK, MESP1, MGP, MGST1, MRPS30, MRPS30-DT, MS4A7, MT-ATP8, NOVA1, PEG10, PHGR1, PI15, PIP, PLAAT4, PLAT, PRSS23, PSD3, PVALB, RAMP1, RBP1, RHOBTB3, SCGB3A1, SCUBE2, SEMA3C, SERPINA1, SH3BGRL, SLC39A6, SLC40A1, SNCG, STC2, TCEAL4, TCIM, TFF1, TFF3, TIMP1, TMC5, TPM1, TPRG1, VSTM2A, VTCN1, WFDC2, XBP1, and ZFP36L1; or
(b) a gene element 2 group (GE2) when the gene set having highest gene expression comprises at least one gene, at least 5 genes, at least 10 genes, at least 15 genes, or at least 20 genes selected from the group consisting of ALDH3B2, ALOX15B, APOD, AZIN1, B2M, BNIP3, C1orf21, CALD1, CALU, CAPG, CD24, CD59, CD74, CD99, CDKN2B, CFD, CKB, CLDN3, CLDN4, CNN3, COL12A1, COX6C, CRIP1, CSRP1, CSRP2, CTNNB1, CTTN, CYSTM1, DDIT4, DHRS2, DD<5, DSC2, EFHD1, EFNA1, ELF5, EN01, FAM229B, FASN, GJA1, GRIK1-AS1, GSTP1, H2AJ, HILPDA, HNRNPH1, HSPA5, IFI27, IFITM3, IGKC, JPT1, KCNC2, KRT15, KRT23, KRT7, LAPTM4B, LDHB, LM04, LTF, MAFB, MAL2, MAOB, MFAP2, MGST1, MRPL15, MT1X, MUCL1, MYBPC1, NME2, NUPR1, PCSK1N, PFN2, PHGDH, PRSS23, PSMB3, PTHLH, PTPN1, RAMP1, RAMP3, RBP1, RSU1, S100A 10, S100A6, SCUBE2, SFRP1, SH3BGRL, SLC39A4, SLC40A1, SOX4, STC2, STOM, TCIM, TFF3, TMSB4X, TTYH1, TUBA1A, UBE2V2, VIM, YBX1, YBX3, YWHAH, and YWHAZ; or
(c) a gene element 3 group (GE3) when the gene set having highest gene expression comprises at least one gene, at least 5 genes, at least 10 genes, at least 15 genes, or at least 20 genes selected from the group consisting of A2M, ACTA2, ACTG2, ANGPTL4, ANXA1, APOD, APOE, BGN, C6ORF15, CALD1, CALML5, CAV1, CAVIN1, CAVIN3, CCL28, CCN2, CD24, CDKN2A, CHI3L1, COL1A2, COL6A 1, COL6A2, COTL1, CRYAB, CSTA, CXCL2, DEFB1, DEPP1, EFEMP1, FABP5, FBXO32, FDCSP, FGFBP2, FN1, GABRP, GSTP1, HLA-A, HLA-B, ID1, IFI27, IGFBP3, IGFBP5, IGFBP7, IL32, KLK5, KLK7, KRT14, KRT15, KRT16, KRT17, KRT5, KRT6A, KRT6B, KRT81, LAMB3, LCN2, LTF, LY6D, MFAP5, MFGE8, MGP, MIA, MMP7, MT1X, MT2A, MYL9, MYLK, NDRG1, NDUFA4L2, NFKBIA, NNMT, PDLIM4, PLS3, POSTN, PRNP, PTN, RARRES1, RCAN1, RGS2, S100A2, S100A4, S100A6, S100A8, S100A9, SAA1, SAA2, SBSN, SERPING1, SFRP1, SGK1, SLC25A37, SLPI, SPARC, SPARCL1, TAGLN, THBS1, TPM2, TSHZ2, VIM, and ZFP36L2; or
(d) a gene element 4 group (GE4) when the gene set having highest gene expression comprises at least one gene, at least 5 genes, at least 10 genes, at least 15 genes, or at least 20 genes selected from the group consisting of ANLN, ANP32E, ARL6IP1, ASF1B, ASPM, ATAD2, AURKA, BIRC5, BUB1B, CCNB1, CCNB2, CDC20, CDC6, CDCA3, CDCA8, CDK1, CDKN2A, CDKN3, CENPA, CENPE, CENPF, CENPK, CENPM, CENPU, CENPW, CIP2A, CKAP2, CKLF, CKS1B, CKS2, CTHRC1, DEK, DLGAP5, DTYMK, DUT, ECT2, FAM111A, FAM111B, GGH, GTSE1, H1-2, H1-3, H2AZ1, H2AZ2, H2BC11, H4C3, HELLS, HMGB1, HMGB2, HMGB3, HMGN2, HMMR, IQGAP3, KIF20B, KIF23, KIF2C, KNL1, KPNA2, LGALS1, MAD2L1, MKI67, MT2A, MYBL2, MZT1, NEK2, NUF2, NUSAP1, PBK, PCLAF, PCNA, PLK1, PRC1, PRR11, PTTG1, RACGAP1, RAD21, RHEB, RNASEH2A, RPL39L, RRM2, SMC4, SPC25, STMN1, TFDP1, TK1, TMEM106C, TMPO, TOP2A, TPX2, TROAP, TTK, TUBA1B, TUBA1C, TUBB, TUBB4B, TYMS, UBE2C, UBE2S, UBE2T, and ZWINT; or
(e) a gene element 5 group (GE5) when the gene set having highest gene expression comprises at least one gene, at least 5 genes, at least 10 genes, at least 15 genes, or at least 20 genes selected from the group consisting of AIF1, ALOX5AP, ANXA1, APOC1, APOE, AREG, C1ORF162, C1QA, C1QB, C1QC, CARD16, CCL3, CCL4, CCL5, CD2, CD27, CD37, CD3D, CD3E, CD48, CD52, CD53, CD69, CD7, CD74, CD83, CELF2, COL1A2, CORO1A, CREM, CST7, CTSL, CTSW, CXCR4, CYBB, CYTIP, DUSP2, EMP3, FCER1G, FN1, FYB1, GIMAP7, GMFG, GPR183, GPSM3, GZMA, GZMK, HOST, HLA-DPA1, HLA-DPB1, HLA-DQA1, HLA-DRA, HLA-DRB1, IGSF6, IL2RG, IL32, IL7R, ISG15, ITGB2, KLRB1, LAPTM5, LCK, LIMD2, LSP1, LST1, LTB, L, Y96, LYZ, MEF2C, MNDA, MS4A6A, MSR1, NKG7, PTPRC, RAC2, RGCC, RGS1, RGS2, RNASE1, S100A4, S100A6, SEPTIN6, SLC2A3, SMAP2, SOCS1, SPARC, SPP1, SRGN, STK4, TMSB4X, TNFAIP3, TRAC, TRBC1, TRBC2, TREM2, TYROBP, VIM, WIPF1, ZEB2, and ZNF331; or
(f) a gene element 6 group (GE6) when the gene set having highest gene expression comprises at least one gene, at least 5 genes, at least 10 genes, at least 15 genes, or at least 20 genes selected from the group consisting of ADIRF, ANAPC11, ATP5ME, AZGP1, BLVRB, BST2, CALM1, CCND1, CD9, CETN2, CISD3, CLDN7, COX6C, CRABP2, CRACR2B, CRIP1, CRIP2, CSTB, CYB5A, CYBA, CYC1, DBI, DCXR, DSTN, EEF1B2, ELOC, EMP2, FXYD3, GPX4, GSTM3, H2AJ, H2AZ1, HINT1, HMGB1, HSPE1, IDH2, JPT1, KDELR2, KRT10, KRT18, KRT19, KRT7, KRT8, LGALS1, LGALS3, LSM3, LSM4, LY6E, MARCKSL1, MIEN1, MIF, MPC2, MRPL12, MRPL51, MRPS34, MTDH, MUCL1, NDUFB9, NDUFC2, NME1, PAFAH1B3, PFDN2, PFN1, PIP, POLR2K, PPDPF, PSMA7, PSMB3, PSME2, RAN, RANBP1, RBIS, REEP5, ROM01, RPS26, S100A14, S100A 16, SEC61G, SELENOP, SH3BGRL, SLC9A3R1, SMIM22, SNRPB, SNRPG, SPINT2, SQLE, SRP9, STARD10, TCEAL4, TMC01, TMEM14B, TPI1, TPM1, TSPAN13, TUBA1B, TUBB, UQCRQ, XBP1, YBX1, and ZNF706; or
(g) a gene element 7 group (GE7) when the gene set having highest gene expression comprises at least one gene, at least 5 genes, at least 10 genes, at least 15 genes, or at least 20 genes selected from the group consisting of AC093001-1, ADIRF, AGR2, AGR3, ANKRD37, APOD, AQP3, ARC, AREG, ATF3, AZGP1, BAMBI, BTG1, BTG2, C15ORF48, CALML5, CCDC74A, CCN1, CD55, CDKN1A, CEBPB, CEBPD, CFD, CLDN3, CLDN4, CST3, CTD-3252C9-4, CTSK, DHRS2, DNAJB1, DUSP1, EDN1, EGR1, ELF3, ELOVL2, ESR1, FHL2, FOS, FOSB, GATA3, GDF15, GRB7, GSTM3, H1-2, HES1, ICAM1, ID2, IER2, IER3, IFITM1, IGFBP4 IGFBP5, IRF1, JUN, JUNB, KLF4, KLF6, KRT15, KRT18, LGALS3, MAFB, MAGED2, MGP, NAMPT, NCOA7, NFKBIA, NFKBIZ, NR4A1, NR4A2, PERP, PLAT, PMAIP1, PRSS23, REL, RHOV, RND1, SWOP, SAT1, SLC39A6, SLC40A1, SOCS3, SOX4, SOX9, STC2, TACSTD2, TCIM, TFF1, TIMP3, TM4SF1, TNFRSF12A, TSC22D3, TUBA 1A, VASN, VEGFA, VTCN1, XBP1, ZFAND2A, ZFP36, ZFP36L1, and ZFP36L2; or
(h) a gene element 8 group (GE8) when the gene set having highest gene expression comprises at least one gene, at least 5 genes, at least 10 genes, at least 15 genes, or at least 20 genes selected from the group consisting of ADIRF, AFF3, ALCAM, ANKRD30A, ANXA2, AR, ARFGEF3, ASAH1, ATP1B1, AZGP1, BTG1, CD59, CDK12, CEBPD, CLDN3, CLDN4, CLTC, CLU, CNN3, CTNNB1, CTNND1, EFHD1, EGR1, ELF3, EPCAM, ERBB2, ESR1, EVL, FOSB, GATA3, GRB7, H4C3, HES1, HLA-B, HNRNPH1, HSPA1A, HSPA1B, IGFBP5, INTS6, ITGB1, ITGB6, ITM2B, JUN, KLF6, KRT7, LDLRAD4, LMNA, LRATD2, MAGED2, MAL2, MARCKS, MT-ND4L, MT2A, MUC1, MYH9, NEAT1, NFIB, PERP, PKM, PLAT, PMEPA1, PSAP, RAD21, RBP1, RHOB, RUNX1, SWOAW, SAT1, SCARB2, SCD, SDC1, SERHL2, SH3BGRL3, SHISA2, SLC38A2, SLC39A6, SLC40A1, SOX4, SYTL2, TACSTD2, TCAF1, TCIM, TFAP2B, TIMP1, TM4SF1, TMC5, TMEM123, TPM1, TRPS1, TSC22D1, TSPYL1, TUBA1A, VEGFA, WSB1, XIST, YBX1, YBX3, ZFP36L1, ZFP36L2, and ZNF292; or
(i) a gene element 9 group (GE9) when the gene set having highest gene expression comprises at least one gene, at least 5 genes, at least 10 genes, at least 15 genes, or at least 20 genes selected from the group consisting of AC093001-1, ADIRF, AGR2, AGR3, APOD, AQP1, AQP5, AREG, ASCL1, AZGP1, BMPR1B, C15ORF48, CALML5, CCL28, CD55, CEACAM6, CFD, CLIC3, CLU, COX6C, CSTB, CTSD, CXCL14, CXCL17, DHRS2, DSCAM-AS1, DUSP1, ERBB2, FADS2, FAM3D, FHL2, GDF15, GLYATL2, GPX1, GSN, GSTP1, HDC, HSPB1, IGFBP5, ISG20, ITM2A, KRT23, KRT7, LGALS1, LGALS3, LY6E, MARCKS, MFGE8, MGP, MS4A7, MT-ATP8, MTCO2P12, MUC5B, MUCL1, NDRG2, NFKBIZ, NPW, NR4A 1, NUDT8, PALMD, PDZK1IP1, PERP, PHGR1, PIP, PLAT, PRSS21, PSCA, PTHLH, PYDC1, RGSW, RGS2, RHCG, RP11-53019-2, S100A 1, SWOAW, S100A6, S100A7, S100A8, S100A9, SWOP, SAA2, SCGB1D2, SCGB2A1, SCGB2A2, SDC2, SERHL2, SERPINA1, SLC12A2, SLC18A2, SLPI, SYNM, TACSTD2, TFF1, TFF3, TM4SF1, TMC5, TSC22D3, TSPAN1, TXNIP, and XBPI; or
(i) a gene element 10 group (GE10) when the gene set having highest gene expression comprises at least one gene, at least 5 genes, at least 10 genes, at least 15 genes, or at least 20 genes selected from the group consisting of AGR2, APOD, AREG, AZGP1, B2M, BST2, BTG2, C15ORF48, CCL20, CD74, CEBPD, CHI3L1, CHI3L2, CP, CRISP3, CSTA, CTSC, CTSD, CTSS, CXCL1, CXCL17, CYBA, DEFB1, FDCSP, GBP1, GBP2, HLA-A, HLA-B, HLA-C, HLA-DMA, HLA-DPA1, HLA-DPB1, HLA-DQA1, HLA-DQA2, HLA-DQB1, HLA-DRA, HLA-DRB1, HLA-DRB5, HLA-E, ID3, IFI16, IFI27, IFI44L, IFI6, IFIT1, IFIT2, IFIT3, IFITM1, IFITM2, IFITM3, IGFBP7, IL32, IRF1, ISG15, KRT15, KRT19, KRT5, KRT7, LCN2, LGALS1, LGMN, LTF, LUM, LY6D, LYZ, MAFB, MARCKS, MGP, MIA, MMP7, MRPS30-DT, MX1, NNMT, PI3, PIGR, RAMP2, RARRES1, RHCG, RNASE1, RSAD2, S100A8, S100A9, SWOP, SAA2, SCGB1D2, SCGB2A1, SERPING1, SLC39A6, SOD2, SPATS2L, TCIM, TFF1, TFF3, TMEM45A, TNFAIP6, TNFSFW, TXNIP, WFDC2, XBP1, and ZFP36.

3-11. (canceled)

12. A method of determining a level of intra-tumoral heterogeneity (ITH) in a tumor, the method comprising classifying a plurality of cancer epithelial cells in the tumor according to the method of claim 2.

13. A method of classifying an immune cell to an immune cell subset, the method comprising:
(i) obtaining a set of expressed genes in the immune cell;
(ii) determining expression levels of genes in a plurality of gene sets and ranking the expression levels in each gene set to identify a gene set having highest gene expression; and
(iii) assigning the immune cell to an immune cell subset corresponding to the gene set having highest gene expression.

14. The method of claim 13, wherein;
(a) the immune cell is a natural killer cell (NK cell) and the immune cell subset is a NK cell subset, wherein the natural killer cell is classified to an NK-0 subset, when the gene set having highest gene expression comprises at least one gene selected from the group consisting of FCGR3A, PRF1, FGFBP2, GZMH, and ETS1; and
(b) the natural killer cell is classified as an NK-1 cell when the gene set having highest gene expression comprises at least one gene selected from the group consisting of NR4A1, NR4A2, DUSP1, DUSP2, FOS, and JUN; or
(c) the natural killer cell is classified as an NK-2 cell when the gene set having highest gene expression comprises at least one gene selected from the group consisting of FCGR3A, PRF1, FGFBP2, GZMA, GZMB, CXCF1, SPON2, CX3CR1, and S1PR5; or
(d) the natural killer cell is classified as an NK-3 cell when the gene set having highest gene expression comprises at least one gene selected from the group consisting of GZMK, SELL IL7R, and LTB; or
(e) the natural killer cell is classified as an NK-4 cell when the gene set having highest gene expression comprises at least one gene selected from the group consisting of ISG15, IFI6, IFIT3, and IFI44L; or
(f) the natural killer cell is classified as an NK-5 cell when the gene set having highest gene expression comprises at least one gene selected from the group consisting of CCL5, HLA-DRB1, KLRC1, CD74, MYADM, and HSPE1; or
(g) the natural killer cell is classified as a reprogrammed NK cell (rNK cell), when the gene set having highest gene expression comprises at least one gene selected from the group consisting of ABCA1, ALOX12, CALD1, CAVIN2, CCL4, CLU, CMKLR1, CR2, CX3CR1, DTX1, DUSP1, F5, FAM81A, FOS, FOSB, GAS2L1, GFRA2, GP6, HEATR9, HES1, ITGAX, JUN, KLRG1, LTBP1, MID1, MPIG6B, NHSL2, NR4A1, NR4A2, NR4A3, NYLK, PARVB, PLXNA4, RASGRP2, RHPN1, SCD, SLC6A4, SLC7A5, THBS1, TMTC1, TNFAIP3, TUBB1, VWF, XDH.

15-20. (canceled)

21. A method of determining a level of interaction between a tumor and a secondary cell population, the method comprising:
(a) obtaining a population of cancer epithelial cells from the tumor;
(b) assigning each cancer epithelial cell obtained in (a) to a gene element group according to the method of claim 1;
(c) determining average expression of each gene element group in (b) across the population of cancer epithelial cells;
(d) obtaining a set of prioritized receptor-ligand pairs across the tumor and the secondary cell population, each comprising a ligand expressed by a cancer epithelial cell assigned in step (b) and a prioritized receptor expressed by a secondary cell;
(e) determining average expression of prioritized receptors from the set of prioritized receptor-ligand pairs in the secondary cell population; and
(f) determining the level of interaction between the tumor and the secondary cell population based on the average expression of each gene element group in (c) and the average expression of prioritized receptors in (e).

22. The method of claim 21, wherein:
(i) the prioritized receptor-ligand pairs in (d) either increase or decrease an interaction between the cancer epithelial cell and the secondary cell and the gene element groups in (b) are further classified as “activating” or “inactivating” based on ligand expression of cells assigned to each gene element group, wherein;
(1) cells assigned to an activating gene element group express ligands from prioritized receptor-ligand pairs that increase the level of interaction between the cancer epithelial cell and the secondary cells, and
(2) cells assigned to an inactivating gene element group express ligands from prioritized receptor-ligand pairs that decrease the level of interaction between the cancer epithelial cell and the secondary cell; and
(ii) determining the level of interaction in (f) is positively weighted by average expression of gene element group classified as “activating” and negatively weighted by average expression of gene element groups classified as “inactivating”.

23. (canceled)

24. The method of claim 22 wherein (f) is calculated using an equation comprising:
$$IP ( {R}_{i} ) = {\sum }_{i = 1}^{10} ( {e}_{i} ) ( {R}_{i} ) ( w )$$
wherein i corresponds to each gene element group, e; is average expression of each gene element group, Rᵢ is the number of prioritized receptors on the secondary cell type, and w is positive 1 for an activating gene element group and negative 1 for an inactivating gene element group.

25. The method of claim 21, wherein the level of interaction in (f) is further based on average levels of one or more interacting factors associated with the cancer epithelial cell population and/or the secondary cell population and the method further comprises (i) determining the average level of at least one interacting factor and (ii) reclassifying the gene element groups in (b) as “activating” or “inactivating” wherein (1) cells classified in an activating gene element group are directly or indirectly acted upon by the interacting factor such that the level of interaction between the cancer epithelial cell and the secondary cells increases as levels of the interacting factor increase and (2) cells classified in an inactivating gene element group are directly or indirectly acted upon by the interacting factor such that the level of interaction between the cancer epithelial cell and the secondary cells decreases as levels of the interacting factor increase.

26. (canceled)

27. The method of claim 25, wherein an interacting score for determining the level of interaction in (f) is calculated using an equation comprising:
$$IP = IP ( {R}_{i} ) + IP ( {B}_{i} )$$
wherein IP(Rᵢ) is calculated as
$$IP ( {R}_{i} ) = {\sum }_{i = 1}^{10} ( {e}_{i} ) ( {R}_{i} ) ( w )$$
wherein i corresponds to each gene element group, eᵢ is average expression of each gene element group, Rᵢ is the number of prioritized receptors on the secondary cell type and w is positive 1 for an activating gene element group and negative 1 for an inactivating gene element group,
and IP(Bᵢ) is calculated as:
$$IP ( {B}_{i} ) = {\sum }_{i = 1}^{10} ( {e}_{i} ) ( {B}_{i} ) ( {w}_{Bi} ) \underline{,}$$
wherein i corresponds to each gene element group, eᵢ is average expression of each gene element group, Bᵢ is the average level of an interacting factor associated with the secondary cell population and w_{Bi} is positive 1 for an activating gene element group and negative 1 for an inactivating gene element group.

28. The method of claim 27, wherein the level of interaction in (f) based on average levels of more than one interacting factors associated with the secondary cell population and the level of interaction in (f) is calculated using an equation comprising:
IP=IP(Rᵢ)+IP(B1ᵢ)+IP(B2ᵢ), wherein each IP(Bᵢ) corresponds to each of the more than one interacting factor.

29. (canceled)

30. The method of claim 25, wherein the one or more interacting factors comprise an autocrine factor, a paracrine factor, a juxtacrine factor, or an endocrine factor.

31. (canceled)

32. The method of claim 21, wherein the secondary cell population comprises an immune cell, a fibroblast, or an endothelial cell.

33-34. (canceled)

35. A method of determining whether a subject with a tumor is a candidate for an immunotherapy, the method comprising:
(a) determining a level of interaction between the tumor in the subject and a secondary cell population; and
(b) determining that the subject is a candidate for the immunotherapy if the level of interaction determined in (a) exceeds a threshold;
wherein the level of interaction between the tumor cells and the secondary cell population in (a) is determined by a method comprising:
(i) obtaining a population of cancer epithelial cells from the tumor;
(ii) assigning each cancer epithelial cell obtained in (i) to a gene element group according to the method of claim 1;
(ii) determining average expression of each gene element group in (ii) across the population of cancer epithelial cells);
(iii) obtaining a set of prioritized receptor-ligand pairs across the tumor and the secondary cell population, each comprising a ligand expressed by a cancer epithelial cell assigned in step (ii) and a prioritized receptor expressed by a secondary cell;
(iv) determining average expression of prioritized receptors from the set of prioritized receptor-ligand pairs in the secondary cell population; and
(v) determining the level of interaction between the tumor and the secondary cell population based on the average expression of each gene element group in (iii) and the average expression of prioritized receptors in (iv).

36. The method of claim 35, wherein the secondary cell population is targeted by the immunotherapy or interacts with a cell targeted by the immunotherapy.

37. A method of treating a patient with a tumor, the method comprising:
(a) determining the subject is a candidate for immunotherapy; and
(b) administering an immunotherapy to the subject,
wherein the patient is determined to be a candidate for immunotherapy if a level of interaction between the tumor in the subject and a secondary cell population exceeds a threshold and wherein the level of interaction between the tumor and the secondary cell population is determined by a method comprising:
(i) obtaining a population of cancer epithelial cells from the tumor;
(ii) assigning each cancer epithelial cell obtained in (i) to a gene element group according to the method of claim 1;
(ii) determining average expression of each gene element group in (ii) across the population of cancer epithelial cells);
(iii) obtaining a set of prioritized receptor-ligand pairs across the tumor and the secondary cell population, each comprising a ligand expressed by a cancer epithelial cell assigned in step (ii) and a prioritized receptor expressed by a secondary cell;
(iv) determining average expression of prioritized receptors from the set of prioritized receptor-ligand pairs in the secondary cell population; and
(v) determining the level of interaction between the tumor and the secondary cell population based on the average expression of each gene element group in (iii) and the average expression of prioritized receptors in (iv).

38. The method of claim 37, wherein the immunotherapy is a T-cell directed therapy, an engineered cellular therapy, a small molecule inhibitor, a cytokine or hormone, an antibody-drug conjugate, a bi-specific antibody or a tri-specific antibody.

39-42. (canceled)

43. The method of claim 37, wherein the tumor is a solid malignant tumor and/or a breast cancer tumor; and/or wherein the subject is a canine or human.

44-45. (canceled)

46. A method for developing an immunotherapy, the method comprising:
(a) obtaining one or more candidate cell sets for targeting with a potential immunotherapy, each candidate cell set comprising a population of tumor cells and a population of secondary cells that interact with or are suspected of interacting with the tumor cells;
(b) determining a level of interaction between the tumor cells and the secondary cells in each candidate set; and
(c) selecting a cell set having a level of interaction that exceeds a threshold for further development of an immunotherapy that alters or exploits the level of interaction between the cell populations in the selected set,
wherein the level of interaction between the tumor cells and the secondary cells in (b) is determined by a method comprising:
(i) obtaining a population of cancer epithelial cells from the tumor;
(ii) assigning each cancer epithelial cell obtained in (i) to a gene element group according to the method of claim 1;
(ii) determining average expression of each gene element group in (ii) across the population of cancer epithelial cells);
(iii) obtaining a set of prioritized receptor-ligand pairs across the tumor and the secondary cell population, each comprising a ligand expressed by a cancer epithelial cell assigned in step (ii) and a prioritized receptor expressed by a secondary cell;
(iv) determining average expression of prioritized receptors from the set of prioritized receptor-ligand pairs in the secondary cell population; and
(v) determining the level of interaction between the tumor and the secondary cell population based on the average expression of each gene element group in (iii) and the average expression of prioritized receptors in (iv).

47. The method of claim 46, wherein the population of secondary cells in each candidate cell set comprises immune cells and the immunotherapy is developed to increase activity of the immune cell population in the selected cell set and/or reduce immune suppression by the tumor cells in the selected cell set.

48. (canceled)
